(19) United States
(12) Patent Application Publication
(10) Pub. No.: US 2024/0280568 A1
(43) Pub. Date: Aug. 22, 2024

(54) AFFINITY REAGENTS HAVING ARTIFICIAL POLYMER SCAFFOLDS

(71) Applicant: Nautilus Subsidiary, Inc., Seattle, WA (US)

(72) Inventors: Seok Ki CHOI, Palo Alto, CA (US); Julia K. ROBINSON, Portola Valley, CA (US); Parag MALLICK, San Mateo, CA (US)

(21) Appl. No.: 18/438,973

(22) Filed: Feb. 12, 2024

Related U.S. Application Data
(60) Provisional application No. 63/581,073, filed on Sep. 7, 2023.
(60) Provisional application No. 63/484,670, filed on Feb. 13, 2023.

(51) Int. Cl. G01N 33/543; G01N 33/533

(52) U.S. Cl. CPC G01N 33/54353; G01N 33/533

(57) ABSTRACT

An affinity reagent, including (a) an artificial polymer comprising a branched chain; (b) an affinity moiety attached to the artificial polymer; and (c) a label attached to the artificial polymer. Also provided are methods for making and using the affinity reagent.

### CROSS-REFERENCE TO RELATED APPLICATIONS

This application claims priority to U.S. Provisional Application No. 63/484,670, filed on Feb. 13, 2023 and U.S. Provisional Application No. 63/581,073, filed on Sep. 7, 2023, each of which applications are incorporated herein by reference in their entirety.

### BACKGROUND

Single-molecule assays hold the promise of identifying and characterizing unique properties of individual biological components that are lost in standard assays that observe average characteristics for ensembles of molecules. For example, a single-molecule binding assay can, at least hypothetically, permit evaluation of unique structural and functional characteristics of individual analytes that are distinguished by their ability to bind to particular affinity reagents. Single-molecule assays also provide for miniaturization and increased throughput when characterizing biological systems having a complex variety of individual species. For example, single-molecule assays have the potential to facilitate analysis on a scale that facilitates research studies and clinical tests performed at the genomic, transcriptomic, proteomic, and metabolomic scale.

Unfortunately, single-molecule assays create unique impediments that do not necessarily impact ensemble-based assays and thus remain unresolved. For example, single-molecule binding assays create demands for labelling bound complexes for high intensity signal generation and/or developing detection hardware capable of very high sensitivity detection. Moreover, affinity reagents having relatively low avidity are adequate for ensemble-based detection since at least some will be present in detectable complexes during signal acquisition. However, when detecting at single-molecule resolution, low avidity can manifest as increased apparent stochasticity or erroneously low apparent binding.

What is needed in the art are improved affinity reagents and methods for detecting, characterizing, or manipulating molecules, especially at single-molecule resolution. The present disclosure satisfies this need and provides other advantages as well.

### SUMMARY

The present disclosure provides a molecule including (a) an artificial polymer having a branched chain; (b) an affinity moiety attached to the artificial polymer; and (c) a label attached to the artificial polymer. The molecule can be particularly useful as an affinity reagent such as a molecular probe. The affinity moiety is optional. Accordingly, a molecule can include (a) an artificial polymer having a branched chain; and (c) a label attached to the artificial polymer. The label is also optional. Accordingly, a molecule can include (a) an artificial polymer having a branched chain; and (b) an affinity moiety attached to the artificial polymer. The artificial polymer can also be removed or replaced as an option.

The present disclosure further provides a complex including an affinity reagent non-covalently bound to an analyte, wherein the affinity reagent includes (a) an artificial polymer having a branched chain; (b) an affinity moiety attached to the artificial polymer; and (c) a label attached to the artificial polymer, and wherein the analyte is non-covalently bound to the affinity reagent via the affinity moiety. The label is optional. Accordingly, a complex can include an affinity reagent non-covalently bound to an analyte, wherein the affinity reagent includes (a) an artificial polymer having a branched chain; and (b) an affinity moiety attached to the artificial polymer. The artificial polymer can also be removed or replaced as an option.

The present disclosure provides a method of making an affinity reagent. The method can include steps of (a) providing an artificial polymer having a branched chain, wherein at least one chain of the artificial polymer has an alkyne moiety; and (b) reacting a cysteine of an antibody with the alkyne moiety, thereby covalently attaching the antibody to the artificial polymer.

A method of making an affinity reagent can include steps of (a) providing an artificial polymer having a branched chain, wherein at least one chain of the artificial polymer has an alkyne moiety; (b) reacting a cysteine of an antibody with the alkyne moiety, thereby covalently attaching the antibody to the artificial polymer, and (c) reacting a second moiety of the artificial polymer with a label, thereby attaching the label to the artificial polymer. Step (b) can be performed prior to step (c) or, alternatively, step (b) can be performed after step (c). If desired, steps (b) and (c) can be carried out in parallel.

A method of making an affinity reagent can include steps of (a) providing an artificial polymer having a branched chain, wherein at least one chain of the artificial polymer includes a first member of a receptor-ligand pair; (b) providing an affinity component having a second member of the receptor-ligand pair; and (c) binding the first and second members of the receptor-ligand pair, whereby the affinity component forms an affinity moiety of the artificial polymer.

The present disclosure further provides a method of detecting an analyte (e.g., a polypeptide). The method can include steps of (a) providing an affinity reagent having an artificial polymer comprising a branched chain that is attached to an affinity moiety and a label; (b) binding the affinity moiety of the affinity reagent to an analyte; and (c) detecting the label, thereby detecting binding of the affinity reagent to the analyte.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIGS. 1A-1B show structures for exemplary artificial polymers having branched chains.

FIGS. 2A-2C show examples of conjugation chemistry and pertinent building blocks applicable in generating polymer-protein constructs. (A) Conjugation of hydrazine and aldehyde via hydrazone linkage. (B) Strain-promoted azide-alkyne click (SPAAC) chemistry. (C) Conjugation of methyl tetrazine (mTz) and trans-cyclooctene (TCO) via inverse electron demand Diels-Alder (IEDDA) reaction.

FIG. 3 shows structures of functional modification reagents, X-PEG-Y, where X is a functional group such as biotin, 4-formylbenzamide (Ald), 6-hydrazinonicotinate (HyNic) acetone hydrazone, dibenzocyclooctyne (DBCO) and azide, while Y refers to an activated ester comprised of N-hydroxysuccinimide (NHS) or 2,3,5,6-tetrafluorophenol (TFP). Abbreviation: PEG=polyethylene glycol.

FIGS. 4A-4D show exemplary affinity reagents having antibodies attached to artificial polymers, and reactions for their manufacture.

FIG. 5 shows structures of representative reagents for polymer functionalization and luminescent dye labeling.

FIG. 6 shows synthesis of PEI(propargyl)ₙ₌₁₀(azide)ₙ₌₁₅(AZ488)_{o=0.05} in which PEI polymer is functionalized with alkyne (n eq), azide (m eq) and AZ488 dye (o eq). reagents and conditions: i) 1,1′-carbonyldiimidazole (CDI)-activated propargyl-PEG4 acid (10 eq), azidoacetic acid NHS ester (10 eq), aqueous acetonitrile, room temperature; ii) AZ488 2,3,5,6-tetrafluorophenyl (TFP) ester (0.05 eq), room temperature, 15 h.

FIG. 7 shows synthesis of PEI(DBCO)ₙ₌₁₀(DL650)ₘ₌₁₅ in which PEI polymer is functionalized with (sulfo)DBCO (n eq) and DL650 dye (m eq). reagents and conditions: i) (sulfo)DBCO-PEG4 2,3,5,6-tetrafluorophenyl (TFP) ester (10 eq), DL dye 650 NHS ester (15 eq), methanol, room temperature, 15 h.

FIG. 8 shows synthesis of G3(propargyl)ₙ₌₇(azide)ₘ₌₁₆(AZ488)_{o=0.5} in which G3 PAMAM dendrimer is functionalized with alkyne (n eq), azide (m eq) and AZ488 dye (o eq). reagents and conditions: i) propargyl-PEG4 N-hydroxysuccinimide (NHS) ester (7 eq), azidoacetic acid NHS ester (16 eq), N,N-diisopropyl-N-ethylamine (DIPEA) (32 eq), methanol, room temperature, 15 h; ii) AZ488 2,3,5,6-tetrafluorophenyl (TFP) ester (0.5 eq), methanol, room temperature, 3 h.

FIG. 9 shows a fluorescent image of a polyacrylamide gel loaded with reaction components from synthesis of G3(propargyl)ₙ₌₇(azide)ₘ₌₁₆(AZ488)_{o=0.5} with DBCO-Cy5. Legends: unmodified G3 dendrimer (lane a); unmodified G3 dendrimer+DBCO-Cy5 (5 eq; lane b); lane c to lane h: G3(propargyl)ₙ₌₇(azide)ₘ₌₁₆(AZ488)_{o=0.5}+DBCO-Cy5 (variable eq); 0 eq (lane c), 2 eq (lane d), 5 eq (lane e), 10 eq (lane f), 15 eq (lane g), 20 eq (lane h). Each reaction mixture was shaken at 35° C. for 15 h and analyzed by PAGE gel analysis.

FIG. 10 shows two routes for synthesis of an affinity reagent having a polyethyleneimine polymer attached to an antibody and a plurality of luminescent labels.

FIGS. 11A-11B show conjugation of PEI(propargyl)₁₀(azide)₁₅(AZ488)_{0.5}(“PEI*”) with anti-HPD IgG under a photo thiol-yne condition. (A) A silver-stained image after polyacrylamide gel analysis. (B) A fluorescent image acquired under the Cy5 channel. lane a: IgG (pre-immune) alone; lane b: anti-HPD IgG alone; lane c: TCEP-reduced anti-HPD IgG; lane d: IgG-PEI*(crude); lane e: IgG-PEI*(spin filtered, MWCO 100 kDa); lane f: IgG-PEI*(Cy5 clicked; spin filtered MWCO 100 kDa); lane g: IgG-PEI*(Cy5 clicked; purified by protein A magnetic beads); lane h: Cy5-labeled free PEI* removed after the treatment of protein A beads.

FIGS. 12A-12B show dye labeling of IgG-PEI with PEI(DBCO)₁₀(DL650)₁₅ through SPAAC conjugation. (A) A silver-stained image after polyacrylamide gel analysis. (B) A fluorescent image acquired under the AF647 channel. MW marker (lane a); TCEP-reduced IgG (lane b); IgG-PEI (lane c); IgG-PEI+PEI(DBCO)₁₀(DL650)₁₅ (lane d); PEI(DBCO)₁₀(DL650)₁₅ control (lane e).

FIGS. 13A-13C show conjugation of G3(propargyl)₇(azide)₁₆(AZ488)_{0.5} (“G3*”) with IgG under a photo thiol-yne condition. (A) A schematic for anti-DTR IgG conjugation with G3* and post Cy5 dye conjugation at G3(azide) moieties through SPAAC. (B) A silver-stained image after polyacrylamide gel analysis. (C) A fluorescent image acquired under the AF647 channel. Legends: anti-DTR IgG (lane a); TCEP-reduced anti-DTR IgG (lane b); IgG-G3* (lane c; crude); IgG-G3* (lane d; purified by spin filtration); IgG-G3*+DBCO-Cy5 (lane e; purified by spin filtration); IgG-G3*+DBCO-Cy5 (lane f, additional purification by treatment with protein A beads); Cy5-labeled free G3* dendrimer removed after the treatment of protein A beads (lane g).

FIG. 14 shows a generic structure of an IgG-PAMAM(dye) conjugate.

FIG. 15 shows a representative data set for anti-DTV IgG-PEI(Cy5) conjugate binding to its target peptide epitope (DTV) presented on a chip surface by total internal reflection fluorescence (TIRF) microscopy.

FIG. 16 shows a schematic description of PAMAM dendrimer generation 3 (G3) modified with biotin, G3(biotin), and its synthesis. Reagents and conditions. i) biotin-PEG₄ NHS ester (15 eq), DIPEA (40 eq), methanol, 25° C., 18 h; ii) glutaric anhydride (200 eq), DIPEA (500 eq), 25° C., 2 h. Abbreviation: DIPEA=N,N-diisopropyl-N-ethylamine; GA=glutaric acid.

FIG. 17 shows a schematic description of PAMAM dendrimer generation 5 (G5) modified with biotin, G5(biotin), and its synthesis. Reagents and conditions. i) biotin-PEG₄ NHS ester (60 eq), DIPEA (200 eq), methanol, 25° C., 18 h; ii) glutaric anhydride (600 eq), DIPEA (500 eq), 25° C., 2 h. Abbreviation: DIPEA=N,N-diisopropyl-N-ethylamine; GA=glutaric acid.

FIG. 18 shows a schematic description of PAMAM dendrimer generation 3 (G3) modified with 4-formylbenzamide, G3(Ald), and its synthesis. Reagents and conditions. i) glutaric anhydride (x=16 eq), DIPEA (50 eq), methanol, 25° C., 0.5 h; ii) 4-formylbenzamide-PEG₄ TFP ester (y=20 eq), 25° C., 3 h; iii) glutaric anhydride (20 eq), DIPEA (50 eq), methanol, 25° C., 1 h. Abbreviation: DIPEA=N,N-diisopropyl-N-ethylamine; GA=glutaric acid; TFP=2,3,5,6-tetrafluorophenol.

FIG. 19 shows a schematic description of PAMAM dendrimer generation 5 (G5) modified with 4-formylbenzamide, G5(Ald), and its synthesis. Reagents and conditions. i) glutaric anhydride (70 eq), DIPEA (200 eq), methanol, 25° C., 0.5 h; ii) 4-formylbenzamide-PEG₄ TFP ester (50 eq), 25° C., 14 h; iii) glutaric anhydride (500 eq), DIPEA (1000 eq), methanol, 25° C., 2 h. Abbreviation: DIPEA=N,N-diisopropyl-N-ethylamine; GA=glutaric acid; TFP=2,3,5,6-tetrafluorophenol.

FIG. 20A shows a schematic description of PAMAM dendrimer generation 5 (G5) modified with DBCO, G5(DBCO), and its synthesis. Reagents and conditions. i) (sulfo)DBCO-PEG₄ TFP ester (30 eq), DIPEA (200 eq), methanol, 25° C., 18 h; ii) glutaric anhydride (1000 eq), DIPEA (1000 eq), 25° C., 2 h. Abbreviation: DIPEA=N,N-diisopropyl-N-ethylamine; GA=glutaric acid.

FIGS. 20B and 20C show photographs of an SDS-PAGE gel either stained (Imperial Blue, left side) or fluorescently detected (right side) acquired after click conjugation reactions of G5(DBCO) with azide-AF647. Legends: lanes a: unmodified G5 dendrimer; lane b: unmodified G5 dendrimer+azide-AF647 (10 eq); lane c: G5(DBCO); lanes d: G5(DBCO)+azide-AF647 (5 eq); lanes e: G5(DBCO)+azide-AF647 (10 eq); lanes f: G5(DBCO)+azide-AF647 (20 eq); lanes g: G5(DBCO)+azide-AF647 (25 eq); lanes h: azide-AF647 (10 eq).

FIG. 21A shows a schematic description of the synthesis of a binary complex formed between IgG(biotin) and streptavidin(AZ647) and its ternary complex with G5(biotin). Reagents and conditions. ii) IgG, biotin-PEG4 NHS ester (x eq to IgG), borate buffered saline (BBS), pH 8.2, 25° C., 15 h; ii) IgG(biotin)ₓ, SA(647)_{y} (n=2 eq to IgG), phosphate buffered saline, 25° C., 0.5 h; iii) IgG(biotin)-SA(647), G5(biotin)_{z=50}, 25° C., 1 h. Abbreviation: SA(647)=streptavidin tetramer modified with y eq of AZ647 dye; G5(biotin)_{z}=PAMAM dendrimer generation 5 (G5) modified with z eq of biotin.

FIG. 21B shows SEC-HPLC traces of SA(AF647) (upper), anti-DTR IgG(biotin) (middle) and their binary complex (lower). Column: Agilent BIO SEC-5, 1000 Å; Eluent: 1×OB; flow rate=0.3 mL/min.

FIG. 22A shows a schematic description of the synthesis of a dendrimer G5-streptavidin conjugate G5(SA) and its complexation with IgG(biotin) to form an IgG-presenting construct of G5(SA)@IgG. Reagents and conditions. i) AZ dye 647 NHS ester (x=5 eq), borate buffered saline (BBS), 25° C., 0.5 h; then 6-hydrazinonicotinate acetone hydrazone (HyNic) NHS ester (x=2×15 eq), 25° C., 15 h; ii) SA(HyNic)ₓ(647)_{y} (n=6-7 eq to G5), G5(Ald)_{z}, 2-amino-5-methoxybenzoic acid (catalyst), PBS, 25° C., 15 h; iii) G5(SA)n, IgG(biotin), 25° C., 0.5 h. Abbreviation: G5(Ald)=PAMAM dendrimer generation 5 (G5) modified with z eq of benzaldehyde (Ald); IgG(biotin)=IgG modified with biotin.

FIG. 22B shows SEC-HPLC traces of G5(Ald) (upper), SA(HyNic)(AZ647) (middle) and their conjugate G5(SA)₆ (lower). Column: Agilent BIO SEC-5, 1000 Å; Eluent: 1×OB; flow rate=0.3 mL/min.

FIGS. 22C and 22D show photographs of an SDS-PAGE gel either stained (Coomassie channel, 22C) or fluorescently detected (22D) acquired after conjugation reactions of G5(Ald) with SA(HyNic). Legends: lane a: G5(Ald); lane b: SA(HyNic); lane c: G5(Ald)+SA(HyNic) (0.8 eq); lane d: G5(Ald)+SA(HyNic) (1.7 eq); lane e: G5(Ald)+SA(HyNic) (3.4 eq); lane f: G5(Ald)+SA(HyNic) (5.0 eq); lane g: G5(Ald)+SA(HyNic) (6.7 eq). Representative conjugate species are marked with white arrows. Abbreviation: SA(HyNic)=streptavidin(HyNic)(AZ647); m=monomer; d=dimer; o=oligomer.

FIGS. 22E and 22F show photographs of an SDS-PAGE gel either Lumitein™ stained (SYPRO RUBY channel, 22E) or fluorescently detected (22F) of complexes formed between G5(SA)₆ and anti-DTR IgG(biotin) at a variable ratio. Legends: lane a, a′: anti-DTR IgG(biotin); lane b, b′: G5(SA)₆; lane c, c′: G5(SA)₆+IgG(biotin) (1 eq); lane d, d′: G5(SA)₆+IgG(biotin) (2 eq); lane e, e′: G5(SA)₆+IgG(biotin) (3 eq); lane f, f: G5(SA)₆+IgG(biotin) (4 eq). Bands of complexes species are marked with white arrows. Abbreviation: G5=G5(SA)₆; IgG=anti-DTR IgG(biotin); SA=streptavidin; m=monomer; d=dimer.

FIG. 23A shows a schematic description of the synthesis of a dendrimer G5-protein A (PA) conjugate G5(PA) and its complexation with IgG to form an IgG-presenting construct, G5(PA)@IgG. Reagents and conditions. i) AZ dye 647 NHS ester (y=5 eq), borate buffered saline (BBS), 25° C., 0.5 h; then 6-hydrazinonicotinate acetone hydrazone (HyNic) NHS ester (x=2×15 eq), 25° C., 15 h; ii) PA(HyNic)ₓ(647)_{y} (n=3-4 eq to G5), G5(Ald)_{z}, 2-amino-5-methoxybenzoic acid (catalyst), PBS, 25° C., 15 h; iii) G5(PA)n, IgG, 25° C., 0.5 h. Abbreviation: G5(Ald)_{z}=PAMAM dendrimer generation 5 (G5) modified with z eq of benzaldehyde (Ald).

FIG. 23B shows SEC-HPLC traces of G5(Ald) (upper), protein A(HyNic)(AZ647) (middle) and their conjugate G5(PA)₄ (lower). Column: Agilent BIO SEC-5, 1000 Å; Eluent: 1×OB; flow rate=0.3 mL/min.

FIGS. 23C and 23D show photographs of an SDS-PAGE gel either stained (Coomassie channel, 23C) or fluorescently detected (23D) of G5(PA)n=i₄ conjugates that resulted from the conjugation reactions of G5(Ald) with SA(HyNic). Legends: lane a: PA(HyNic); lanes b-f: G5(Ald)+PA(HyNic) (variable eq); lane b: 1 eq; lane c: 2 eq; lane d: 3 eq; lane e: 4 eq; lane f: 5 eq; lane g: G5(Ald). Representative conjugate species are marked with white arrows. Abbreviation: PA=PA(HyNic)=protein A modified with HyNic and AZ647.

FIG. 23E shows SEC-HPLC traces of anti-DTR IgG (upper), G5(PA)₄ (middle) and their complexes G5(PA)₄@IgG formulated at two formulation ratios (lower two traces). Column: Agilent BIO SEC-5, 1000 Å; Eluent: 1×OB; flow rate=0.3 mL/min.

FIG. 24A shows a schematic description of hydrazine-modified antibody IgG(HyNic)(647) and its conjugation with aldehyde-modified dendrimer G5(Ald) to produce G5(IgG)ₙ(n=average valency of IgG per G5). Reagents and conditions. i) IgG, AZ dye 647 NHS ester (y=5 eq), borate buffered saline (BBS), pH 8.2, 25° C., 0.5 h; then 6-hydrazinonicotinate acetone hydrazone (HyNic) NHS ester (x=2×15 eq), 25° C., 15 h; ii) IgG(HyNic)ₓ(647)y, G5(Ald)_{z}, 2-amino-5-methoxybenzoic acid, PBS, 25° C., 15 h. Abbreviation: G5(Ald)_{z}=PAMAM dendrimer generation 5 (G5) modified with z eq of benzaldehyde (Ald).

FIGS. 24B and 24C show photographs of an SDS-PAGE gel either Lumitein™ stained (SYPRO RUBY channel) or fluorescently detected of anti-DTR IgG(HyNic)(647) and its G5(Ald) conjugate products, G5(IgG)ₓ₌₃. This gel analysis was performed in a mini gel of 6% tris-glycine (Novex™; ThermoFisher Scientific). Legends: lane a: ant-DTR IgG; lane b: IgG(HyNic)(AZ647); lane c: G5(Ald); lanes d-g: G5(Ald)+IgG(HyNic) (variable eq); lane d: 0.5 eq; lane e: 1 eq; lane f: 2 eq; lane g: 3 eq. Abbreviation: IgG(HyNic)=IgG modified with HyNic and AZ647.

FIG. 25A shows a schematic description of azide-modified antibody IgG(azide) and its click conjugation with DBCO-modified dendrimer G5(DBCO) to produce G5(IgG)ₙ(n=average valency of IgG per G5). Reagents and conditions. i) IgG, azide-PEG4 NHS ester (x=20 eq to IgG), borate buffered saline (BBS), pH 8.2, 25° C., 15 h; ii) IgG(azide)ₓ, G5(DBCO)_{y}, PBS, 25° C., 15 h. Abbreviation: G5(DBCO)y=PAMAM dendrimer generation 5 (G5) modified with y=30 eq of DBCO.

FIGS. 25B and 25C show photographs of an SDS-PAGE gel either silver stained or fluorescently detected of anti-DTR IgG(azide) and its G5(DBCO) conjugate products, G5(IgG)ₙ₌₃. This gel analysis was performed in a mini gel of 6% tris-glycine (Novex™; ThermoFisher Scientific). Legends: lane a: anti-DTR IgG (unmodified); lane b: IgG(azide)+G5(DBCO) (5:1), then azide-AF647; lane c: IgG(azide)+G5(DBCO) (2:1), then azide-AF647; lane d: IgG(azide)+DBCO-AF647; lane e: G5(DBCO)+azide-AF647. Abbreviation: IgG(azide)=IgG modified with azide residues through amide linkage at lysine; G5(DBCO)=G5 PAMAM dendrimer modified with (sulfo)DBCO residues.

FIGS. 26A-26D show plots of detection efficiency of a dendrimer Lobe (anti-DTR IgG-SA-G5 complex) binding to peptides containing the DTR epitope and presented on a streptavidin-SNAP attached to an array surface. Colocalization rate is adjusted for surface non-specific binding (NSB). Anti-DTR dendrimer lobe refers to a ternary complex of IgG-SA-G5 dendrimer described in FIG. 21A. Legends. (A) 10 nM of anti-DTR dendrimer lobe, (B) 50 nM of anti-DTR dendrimer lobe, (C) 100 nM of anti-DTR dendrimer lobe, (D) 5 nM of anti-DTR origami tile lobe. Test concentrations for dendrimer Lobes refer to the number of antibody molecules per Lobe.

FIG. 27 shows a plot of binding efficiency of two anti-DTR IgG-dendrimer Lobes binding to peptides having DTR (on target) or HSP (off target) epitopes. The peptides are presented on a streptavidin-SNAP measured in a fluorescence-linked immunosorbent assay (FLISA) assay. Each dendrimer Lobe is a complex of either G5(SA)@IgG described in FIG. 22A or that of G5(PA)@IgG described in FIG. 23A. Legends: G5(SA)@anti-DTR IgG(biotin) tested at 100 nM (A) or 50 nM (D); G5(PA)@anti-DTR IgG(biotin) tested at 100 nM (B) or 50 nM (E); anti-DTR origami tile lobe tested at 100 nM (C) or 50 nM (F). Test concentrations for dendrimer Lobes refer to the number of antibody molecules per Lobe.

FIG. 28 shows a plot of binding efficiency of two anti-DTR IgG-dendrimer Lobes (G5(SA)@IgG and G5(IgG)₃) to either a single DTR peptide (on target) or HSP peptide (off target) presented on a TCO-SNAP determined in a fluorescence-linked immunosorbent assay (FLISA) assay. Each dendrimer Lobe carries three anti-DTR IgG molecules per dendrimer on a mean basis, and their structural features are described in FIG. 22A for G5(SA)@IgG or in FIG. 24A for G5(IgG)₃. Test concentrations for dendrimer Lobes refer to the number of antibody molecules per Lobe.

### DETAILED DESCRIPTION

The present disclosure provides affinity reagents having artificial polymers that can provide scaffolds for attachment of one or more functional moiety. For example, an affinity reagent can include an artificial polymer that is attached to at least one affinity moiety, the affinity moiety recognizing an analyte of interest. The artificial polymer can optionally be attached to at least one label moiety, the label moiety being detectable, for example, by producing a measurable signal.

An advantage of using an artificial polymer as a scaffold is the ability to support a plurality of attached affinity moieties. The affinity moieties can recognize the same epitopes, for example, due to the affinity moieties having the same structure as each other. The presence of a plurality of affinity moieties that recognize the same epitope can provide the advantage of increased avidity of the affinity reagent for a target molecule having the epitope. Alternatively, a plurality of affinity moieties that is attached to an artificial polymer can have different structures, thereby recognizing different epitopes. The presence of a plurality of different affinity moieties can increase the diversity of epitopes that are recognized by the affinity reagent. This can be advantageous, for example, in polypeptide binding assays wherein a large number of different polypeptides is to be distinguished using a smaller number of different affinity reagents. Exemplary assays are set forth herein and in U.S. Pat. No. 10,473,654 or U.S. Pat. App. Pub. Nos. 2020/0318101 A1 or 2020/0286584 A1; U.S. patent application Ser. No. 18/045,036, or Egertson et al., BioRxiv (2021), DOI: 10.1101/2021.10.11.463967, each of which is incorporated herein by reference.

Another advantage of using an artificial polymer as a scaffold is the ability to support a plurality of attached label moieties. The label moieties can produce overlapping signals, for example, due to the label moieties having the same structure as each other. The presence of a plurality of label moieties that produce overlapping signals can provide the advantage of increased detectability of the affinity reagent. In some configurations, a variety of different label moieties can be attached to an artificial polymer scaffold of an affinity reagent. The use of color combinations can increase the palette of unique signal characteristics available for distinguishing one affinity reagent from another. For example, a set of only three different affinity reagents can be distinguished from each other if each affinity reagent is attached to only one of the labels; however, if each affinity reagent is labeled with two of the labels, then a set of at least six different affinity reagents can be distinguished from each other.

Terms used herein will be understood to take on their ordinary meaning in the relevant art unless specified otherwise. Several terms used herein and their meanings are set forth below.

As used herein, the term “address” refers to a location in an array where a particular analyte (e.g., polypeptide, or nucleic acid) is present. An address can contain a single analyte (i.e., one and only one analyte), or it can contain a population of several analytes of the same species (i.e., an ensemble of the analyte species). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁸, 1×10¹⁰, 1×10¹², 1×10¹⁴, or more addresses.

As used herein, the term “affinity moiety” refers to a moiety of a molecule or other substance, the moiety being capable of specifically or reproducibly binding to an analyte (e.g., polypeptide). An affinity moiety can be larger than, smaller than, or the same size as the analyte. An affinity moiety may form a reversible or irreversible bond with an analyte. An affinity moiety may bind with an analyte in a covalent or non-covalent manner. An affinity moiety may include a reactive affinity reagent, catalytic affinity reagent (e.g., kinase, protease, etc.) or non-reactive affinity reagent (e.g., antibody or functional fragment thereof). An affinity moiety can be non-reactive and non-catalytic, thereby not permanently altering the chemical structure of an analyte to which it binds. A particularly useful affinity moiety is an antibody, such as a full-length antibody or functional fragment thereof. A functional fragment of an antibody can include any fragment that is capable of binding to an epitope with a detectable affinity, such as a Fab, Fab′, F(ab′)₂, single-chain variable (scFv), di-scFv, tri-scFv, or microantibody. Other useful affinity moieties include, for example, affibodies, affilins, affimers, affitins, alphabodies, anticalins, avimers, DARPins, monobodies, nanoCLAMPs, nucleic acid aptamers, polypeptide aptamers, lectins or functional fragments thereof. A molecule that is capable of specifically or reproducibly binding to an analyte and that can or will form an affinity moiety can be referred to as an “affinity component.”

As used herein, the term “array” refers to a population of analytes (e.g., polypeptides) that are associated with unique identifiers such that the analytes can be distinguished from each other. A unique identifier can be, for example, a solid support (e.g., particle or bead), address on a solid support, tag, label (e.g., luminophore), or barcode (e.g., nucleic acid barcode) that is associated with an analyte and that is distinct from other identifiers in the array. Analytes can be associated with unique identifiers by attachment, for example, via covalent bonds or non-covalent bonds (e.g., ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

As used herein, the term “attached” refers to the state of two things being joined, fastened, adhered, connected or bound to each other. Attachment can be covalent or non-covalent. For example, a label can be attached to a polymer by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “artificial” when used in reference to a substance, means that the substance is made by human activity rather than occurring naturally. For example, a polymer that is made at least in part by human activity is referred to as an “artificial polymer.”

As used herein, the term “branched chain” refers to a chain in a polymer, wherein the chain has at least one branch point. A branched chain can include at least 1, 2, 3, 4, 5, 6, 8 or 10 branch points. Alternatively or additionally, a branched chain can include at most 10, 8, 6, 5, 4, 3, 2 or 1 branch points. A branch point is a covalent intersection between at least two chains. For example, at least 2, 3, 4, 5 or more chains can intersect at a branch point of a branched chain. Alternatively or additionally, at most 5, 4, 3 or 2 chains can intersect at a branch point of a branched chain.

As used herein, the term “click reaction” refers to single-step, thermodynamically-favorable conjugation reaction utilizing biocompatible reagents. A click reaction may be configured to not utilize toxic or biologically incompatible reagents (e.g., acids, bases, heavy metals) or to not generate toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about −5 kiloJoules/mole (kJ/mol), −10 kJ/mol, −25 kJ/mol, −100 kJ/mol, −250 kJ/mol, −500 kJ/mol, or less. Exemplary click reactions may include metal-catalyzed azide-alkyne cycloaddition, strain-promoted azide-alkyne cycloaddition (SPAAC), strain-promoted azide-nitrone cycloaddition, strained alkene reactions, thiol-ene reaction, photo thiol-ene reaction, Diels-Alder reaction, inverse electron demand Diels-Alder reaction (IEDDA), [3+2] cycloaddition, [4+1]cycloaddition, nucleophilic substitution, dihydroxylation, thiol-yne reaction, photo thiol-yne reaction, photoclick, nitrone dipole cycloaddition, norbornene cycloaddition, oxanobornadiene cycloaddition, tetrazine ligation, Staudinger ligation, and tetrazole photoclick reactions. Exemplary reactive moieties utilized to perform click reactions may include alkenes, alkynes, azides, epoxides, amines, thiols, nitrones, isonitriles, isocyanides, aziridines, activated esters, and tetrazines. Other well-known click conjugation reactions may be used having complementary bioorthogonal reaction species, for example, where a first click component comprises a hydrazine moiety and a second click component comprises an aldehyde or ketone group, and where the product of such a reaction comprises a hydrazone functional group or equivalent. Exemplary bioorthogonal and click reactions are set forth in U.S. Pat. App. Pub. No. 2021/0101930 A1, which is incorporated herein by reference.

The term “comprising” is intended herein to be open-ended, including not only the recited elements, but further encompassing any additional elements.

As used herein, the term “dendron” refers to a polymer molecule, or polymeric moiety, having a moiety that constitutes a focal point from which a plurality of branched chains emanate, wherein two or more of the branched chains have the same structure. Accordingly, the path through the covalent structure of each of the two or more branched chains, from the focal point to the distal moiety of the respective branched chain, includes the same number and type of constitutional repeating units. A dendron can have radial symmetry around the focal point. Two or more branched chains of a dendron can have reflectional symmetry with respect to a line drawn through the focal point. In some cases, a dendron can have reflectional symmetry for all branched chains with respect to a line drawn through the focal point. A dendron can include 2, 3, 4, 5, 6 or more branched chains. The term “dendrimer” is used synonymously with the term “dendron” herein.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to an affinity target within a polypeptide or other analyte. Epitopes may include amino acid sequences that are sequentially adjacent in the primary structure of a polypeptide. Epitopes may include amino acids that are structurally adjacent in the secondary, tertiary or quaternary structure of a polypeptide despite being non-adjacent in the primary sequence of the polypeptide. An epitope can be, or can include, a moiety of a polypeptide that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

As used herein, the term “fluid-phase,” when used in reference to a molecule, means the molecule is in a state wherein it is mobile in a fluid, for example, being capable of diffusing through the fluid.

As used herein, the term “immobilized,” when used in reference to a molecule that is in contact with a fluid phase, refers to the molecule being prevented from diffusing in the fluid phase. For example, immobilization can occur due to the molecule being confined at, or attached to, a solid phase. Immobilization can be temporary (e.g., for the duration of one or more steps of a method set forth herein) or permanent. Immobilization can be reversible or irreversible under conditions utilized for a method, system or composition set forth herein.

As used herein, the term “label” refers to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a luminophore (e.g., fluorophore), chromophore, nanoparticle (e.g., gold, silver, carbon nanotubes, quantum dots, upconversion nanocrystals), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the term “moiety” refers to a component or part of a molecule. The term does not necessarily denote the relative size of the component or part compared to the rest of the molecule, unless indicated otherwise. A moiety can include one or more atoms.

As used herein, the term “nucleic acid origami” refers to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structure of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the term “orthogonal,” when used in reference to two or more reactants, refers to the two or more reactants having mutually non-competing reactivity. Similarly, two or more reactions are referred to as being orthogonal when (1) the reagents or products of a first reaction are inert to reagents and products of a second reaction, and/or (2) the reagents or products of the second reaction are inert to reagents and products of the first reaction. An orthogonal reaction may be further characterized as being inert to components of a biological system other than those targeted by the reactants. An orthogonal reaction may utilize an enzymatic reaction, such as attachment between a first molecule and a second molecule by an enzyme such as a sortase, a ligase, or a subtiligase. An orthogonal reaction may utilize an irreversible peptide capture system, such as SpyCatcher/SpyTag, SnoopCatcher/SnoopTag, or SdyCatcher/SdyTag.

As used herein, the term “polymer” refers to a molecule having a plurality of repeating monomer subunits connected via a network of covalent bonds. The network can include one or more chain(s) of the monomer subunits. A polymer can be linear or branched. A linear polymer includes only one chain in the network of covalent bonds. A branched polymer includes at least two chains in the network of covalent bonds. For example, a branched polymer can include at least 2, 3, 4, 5, 6, 8, 10 or more chains in the network of covalent bonds. Alternatively or additionally, a branched polymer can include at most 10, 8, 6, 5, 4, 3 or 2 chains in the network of covalent bonds. A polymer can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds.

As used herein, the term “polypeptide” refers to a molecule comprising two or more amino acids joined by a peptide bond. A polypeptide may also be referred to as a protein, oligopeptide or peptide. A polypeptide can be a naturally-occurring molecule, or synthetic molecule. A polypeptide may include one or more non-natural amino acids, modified amino acids, or non-amino acid linkers. A polypeptide may contain D-amino acid enantiomers, L-amino acid enantiomers or both. Amino acids of a polypeptide may be modified naturally or synthetically, such as by post-translational modifications. In some circumstances, different polypeptides may be distinguished from each other based on different genes from which they are expressed in an organism, different primary sequence length or different primary sequence composition. Polypeptides expressed from the same gene may nonetheless be different proteoforms, for example, being distinguished based on non-identical length, non-identical amino acid sequence or non-identical post-translational modifications. Different polypeptides can be distinguished based on one or both of gene of origin and proteoform state.

As used herein, the term “single,” when used in reference to an object such as an analyte, means that the object is individually manipulated or distinguished from other objects. A single analyte can be a single molecule (e.g., single polypeptide), a single complex of two or more molecules (e.g., a multimeric polypeptide having two or more separable subunits, a single polypeptide attached to a structured nucleic acid particle or a single polypeptide attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array.

As used herein, the term “solid support” refers to a substrate that is insoluble in aqueous liquid. Optionally, the substrate can be rigid. The substrate can be non-porous or porous. The substrate can optionally be capable of taking up a liquid (e.g., due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

As used herein, the term “structured nucleic acid particle” or “SNAP” refers to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same mass or sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as polypeptides, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

The embodiments set forth below and recited in the claims can be understood in view of the above definitions.

The present disclosure provides a molecule including (a) an artificial polymer having a branched chain; (b) an affinity moiety attached to the artificial polymer; and (c) a label attached to the artificial polymer. The molecule can be particularly useful as an affinity reagent such as a molecular probe. The affinity moiety is optional. Accordingly, a molecule can include (a) an artificial polymer having a branched chain; and (c) a label attached to the artificial polymer. The label is also optional. Accordingly, a molecule can include (a) an artificial polymer having a branched chain; and (b) an affinity moiety attached to the artificial polymer. The artificial polymer can also be removed or replaced as an option.

An artificial polymer can provide a relatively large number of sites to which functional moieties, such as affinity moieties and label moieties, are attached. The composition and length of the artificial polymer in an affinity reagent can be configured to provide a number of moieties that provide a desired property or characteristic to the affinity reagent. For example, the number of label moieties can be configured to provide a desired level of detectability. Generally, more label moieties can be included to provide a higher signal intensity compared to use of fewer label moieties. Conversely, it may be beneficial to include fewer label moieties for example to reduce signal intensity or to inhibit unwanted side-effects of using certain types of labels, such as luminophores which can produce unwanted radicals in some situations.

The composition of an artificial polymer can also be configured to provide desired characteristics such as solubility. For example, solubility of an affinity reagent in aqueous solution can generally be increased by use of shorter length polymers, polymers having smaller molecular weights, polymers having more moieties that are polar, or polymers having fewer non-polar moieties. On the other hand, an affinity reagent can include an artificial polymer that is configured to have reduced aqueous solubility, for example, providing particle-like properties that facilitate precipitation of the affinity reagent or attachment of the affinity reagent to a solid support. In some cases, an affinity reagent can be configured for use in organic solvents or non-aqueous fluids by including polymers having more non-polar moieties, or polymers having fewer polar moieties. Optionally, an artificial polymer or affinity reagent of the present disclosure can be configured to lack one or more of the foregoing structures or characteristics.

An artificial polymer can have a linear or branched structure. A branched structure can be advantageous, for example, for providing a relatively compact structure. A compact structure can be useful for confining labels to a relatively small volume of space, thereby increasing localization of signals during detection and improving resolution of neighboring affinity reagents in a given volume of solution or in a given area on a surface. In some cases, a branched structure can provide particle-like characteristics to an affinity reagent to which the artificial polymer is attached. Optionally, an artificial polymer or affinity reagent of the present disclosure can be configured to lack one or more of the foregoing structures or characteristics.

A branched structure can include at least one branchpoint where a first chain attaches to a second chain. A branched chain can include a plurality of branch points, for example, at least 2, 3, 4, 5, 6, 8, 10 or more branch points. Alternatively or additionally, a branched chain can include at most 10, 8, 6, 5, 4, 3, or 2 branch points. The branching of an artificial polymer can be regular or irregular with respect to the location of branch points along a primary chain and/or the composition of the sidechains that branch from the primary chain. Any of a variety of bonds can occur at branch points including, but not limited to, carbon-carbon bonds, ester bonds, amine bonds (e.g., tertiary or secondary amines), amide bonds, or ether bonds. In some configurations a branched structure can also be cross-linked. However, branched structures need not be crosslinked, for example, having thermoplastic characteristics.

A branched structure can be configured as a graft polymer wherein one or more of the side chains are different, structurally or configurationally, from the main chain. Another configuration is a star-shaped structure in which a single branch point gives rise to multiple linear chains. The star-shaped structure can be regular whereby the chains are identical. Alternatively, adjacent chains can be composed of different repeating subunits, whereby the star polymer molecule is variegated. A comb polymer configuration includes a main chain with at least two three-way branch points and linear side chains. If the chains are identical, the comb polymer molecule is said to be regular. Another configuration is a brush polymer structure which consists of a main chain with linear, unbranched side chains. At least one of the branch points in a brush polymer can have four-way functionality or larger. A polymer network is a network in which all polymer chains are interconnected to form a single macroscopic entity by many crosslinks, examples of which include thermosets or interpenetrating polymer networks. A branched structure can include one or more dendrons, for example, forming a dendrimer or other repetitively branched structure. Optionally, an artificial polymer or affinity reagent of the present disclosure can lack one or more of the foregoing structures.

An artificial polymer can be an organic polymer, wherein the skeletal structure of the polymer includes only organic monomer subunits; an inorganic polymer, wherein the skeletal structure does not contain carbon atoms; or an inorganic-organic polymer, wherein the skeletal structure includes organic and inorganic monomer subunits. An artificial polymer can be a homopolymer, for example, being derived from a single species of monomer. Alternatively, an artificial polymer can be a copolymer, for example, being derived from multiple species of monomers. Optionally, an artificial polymer or affinity reagent of the present disclosure can be devoid of one or more of the foregoing structures.

An affinity reagent or artificial polymer can be characterized in terms of molecular weight (or molecular weight distribution) in a desired size range. For example, the molecular weight, average molecular weight distribution, minimum molecular weight distribution or maximum molecular weight distribution can be at least 1 kDa, 2 kDa, 5 kDa, 10 kDa, 25 kDa, 50 kDa or more. Alternatively or additionally, the molecular weight, average molecular weight distribution, minimum molecular weight distribution or maximum molecular weight distribution can be at most 50 kDa, 25 kDa, 10 kDa, 5 kDa, 2 kDa, 1 kDa or less. An affinity reagent or artificial polymer can be characterized in terms of radius of gyration. For example, the radius of gyration can be at least about 2 nm, 5 nm, 10 nm, 15 nm, 25 nm, 50 nm or more. Alternatively or additionally, an affinity reagent or artificial polymer can be configured to have a radius of gyration that is at most about 50 nm, 25 nm, 15 nm, 10 nm, 5 nm, 2 nm or less. An artificial polymer can be characterized in term of degree of polymerization (i.e. number of monomer subunits) present. For example, an artificial polymer can include at least 2, 10, 20, 30, 40, 50, 100, 200, 300 or more monomers. Alternatively or additionally, an artificial polymer can include at most 300, 200, 100, 50, 40, 30, 20, 10, or 2 monomers.

A population of affinity reagents or artificial polymers can be characterized in terms of dispersity. Dispersity can be measured as the ratio of the weight-average molar mass (Mw) and the number-average molar mass (Mₙ): polydispersity=M_{w}/Mₙ. Dispersity can also be measured as the ratio of the weight-average degree of polymerization and the number-average degree of polymerization. A population of affinity reagents or artificial polymers can be relatively uniform (or monodisperse), or the population can be relatively non-uniform (or polydisperse). The dispersity for a population of affinity reagents or artificial polymers can be at least 1.0, 1.2, 1.4, 1.6, 1.8, 2.0, 5, 10 or higher. Alternatively or additionally, the dispersity for a population of affinity reagents or artificial polymers can be at most 10, 5, 2.0, 1.8, 1.6, 1.4, 1.2, 1.0 or lower.

An artificial polymer of the present disclosure can lack natural polymers or monomers found in natural polymers. For example, the skeletal structure of the artificial polymer can lack natural polymers or monomers. This can be the case whether or not the artificial polymer has attached moieties that include natural polymers or monomers. Examples of natural moieties that can be absent from an artificial polymer, for example, in the skeletal structure include, but are not limited to, nucleic acids (e.g., DNA or RNA), nucleotides (e.g., deoxyribonucleotides or ribonucleotides), nucleosides (e.g., deoxyribonucleosides or ribonucleosides), peptides (e.g., proteins, polypeptides or oligopeptides), amino acids, or sugars (e.g,. saccharide monomers, monosaccharides, oligosaccharides, polysaccharides or glycans). An artificial polymer can optionally lack any polymer or monomer that is synthesized in vivo or that is capable of being synthesized in vivo. Alternatively, an artificial polymer can include natural moieties that are combined to form a non-naturally occurring molecule. For example, an artificial polymer can be composed of nucleic acid monomers or nucleic acid strands that form a non-naturally occurring nucleic acid dendrimer structure.

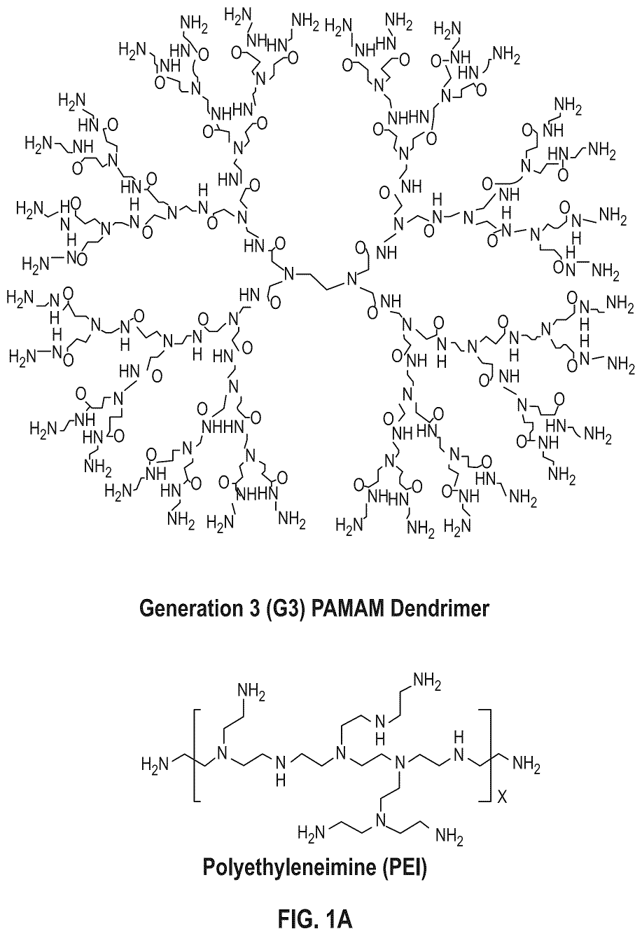

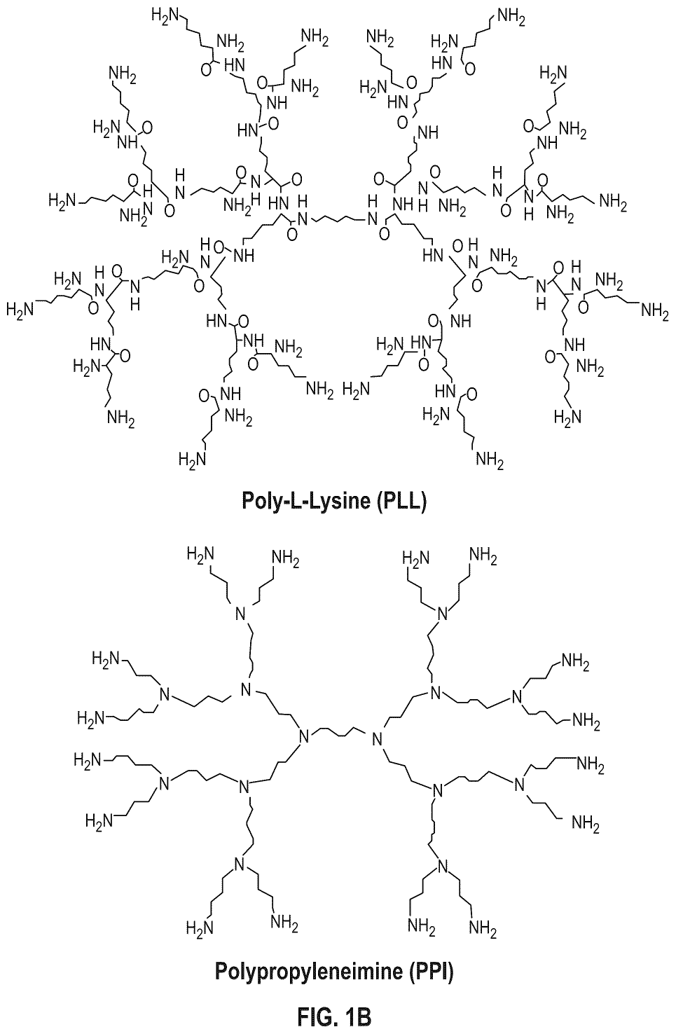

Particularly useful artificial polymers include, for example, poly(amidoamine) (PAMAM) dendrimer, poly(amidoamine) dendron, hyperbranched polymers such as linear and branched polyethyleneimine (PEI) and polypropyleneimine (PPI), star polymers, grafted polymers, peptide-based linear or branched dendrimers such as branched poly-L-lysine (PLL) and silane-cored dendrimer. Exemplary structures are shown in FIG. 1. Other useful artificial polymers include dendrimer nucleic acids having branching structures. See, for example, Liu et al., J. Mater. Chem. B 9:4991-5007 (2021) and Meng et al., ACS Nano 8:6171-6181 (2014), each of which is incorporated herein by reference. Several physicochemical properties of branched artificial polymers can be beneficial to their applications for affinity reagents such as good aqueous solubility, relatively low viscosity, and polymer stability. Useful polymers also offer beneficial features in their structure and three-dimensional architecture such as well-defined shapes and large number of terminal branches which are pre-organized on the surface. For example, the branches can be terminated with a plurality of amines, carboxylic acids, hydroxyls, or combinations thereof. Polymers that carry such functionalities can facilitate specific chemical modifications on their terminal branches which are applicable for affinity reagent development. Examples of such polymer scaffolds are set forth in Tomalia, et al. J Polym Sci Part A: Polym Chem 40: 2719-2728 (2002); Higashihara, et al. Polym J 44, 14-29 (2012); Gupta, et al. J. Phys. Chem. B 124, 20, 4193-4202 (2020); Ren, et al. Chem. Rev. 116, 12, 6743-6836 (2016); Chis, et al. Molecules 25(17): 3982 (2020); Zheng, et al. or Chem. Soc. Rev. 44, 4091-4130 (2015), each of which is incorporated herein by reference.

An artificial polymer can be configured to form a particle. The particle may have any of a variety of sizes and shapes to accommodate use in a desired application. For example, a particle can have a regular or symmetric shape or, alternatively, a particle can have an irregular or asymmetric shape. The shape can be rigid or pliable. The size or shape of a particle can be characterized with respect to volume. For example, a particle can have a minimum, maximum or average volume of at least about 1 um³, 10 um³, 100 um³, 1 mm³, 10 mm³, 100 mm³, 1 cm³ or more. Alternatively or additionally, a particle can have a minimum, maximum or average volume of no more than about 1 cm³, 100 mm³, 10 mm³, 1 mm³, 100 um³, 10 um³, 1 um³ or less. In some configurations, an artificial polymer is not configured to form a particle.

A particle can be characterized with respect to its footprint (e.g., occupied area on a surface). A footprint may have a regular shape or an approximately regular shape, such as triangular, square, rectangular, circular, ovoid, or polygon. Optionally, the minimum, maximum or average area for a particle footprint can be at least about 10 nm², 100 nm², 1 um², 10 um², 100 um², 1 mm² or more. Alternatively or additionally, the minimum, maximum or average area for a particle footprint can be at most about 1 mm², 100 um², 10 um², 1 um², 100 nm², 10 nm², or less.

A particle that is used in accordance with the present disclosure can be suspended in a fluid, immobilized on a solid support, or immobilized in another material such as a gel or solid support material. For example, a population of particles can be colloidal for some, or all steps of a method set forth herein. Alternatively, a population of particles can be immobilized in, or on a solid support, for example, by gravity, non-covalent bonding, covalent bonding, coordination, adhesion or a combination thereof. Optionally, a first particle can be attached to a second particle. The first and second particles can compose the same material as each other or different materials from each other.

Any of a variety of affinity moieties can be used in a composition or method set forth herein including, but not limited to, those exemplified herein. In some configurations, a composition or method set forth herein can lack one or more of the affinity moieties set forth herein.

An antibody is a particularly useful affinity moiety for use in a composition or method set forth herein. The antibody can be any antigen-binding molecule or molecular complex having at least one complementarity determining region (CDR) that binds to or interacts with a particular antigen with high affinity. An antibody can include four polypeptide chains: two heavy chains (HC1 and HC2) and two light chains (LC1 and LC2). HC1 and HC2 can be covalently connected by one, two or more disulfide bonds. HC1 can be covalently connected to LC1 by at least one disulfide bond. HC2 can be covalently connected to LC2 by at least one disulfide bond. Each heavy chain can include a heavy chain variable region (V_{H}) and a heavy chain constant region (C_{H}). The heavy chain constant region can include three domains, C_{H1}, C_{H2} and C_{H3}. Each light chain can include a light chain variable region (V_{L}) and a light chain constant region (C_{L}). The V_{H} and V_{L} regions can further include regions of hypervariability, termed complementarity determining regions (CDRs), interspersed with regions that are more conserved, termed framework regions (FR). Each V_{H} and V_{L} can include three CDRs and four FRs, arranged from amino-terminus to carboxy-terminus in the following order: FR1, CDR1, FR2, CDR2, FR3, CDR3, FR4.

An antibody can include all elements of a full-length antibody, such as those enumerated above. However, an antibody need not be full length and functional fragments can be particularly useful for many uses. The term “antibody” as used herein encompasses full length antibodies and functional fragments thereof. A functional fragment can be naturally occurring, enzymatically obtainable, synthetic, or genetically engineered. An antibody can be obtained using any suitable technique such as proteolytic digestion or recombinant genetic engineering techniques involving the manipulation and expression of DNA encoding one or more antibody domains. Such DNA is readily available, for example, from commercial sources, DNA libraries (e.g., phage-antibody libraries), or can be synthesized. The DNA may be manipulated chemically or by using molecular biology techniques, for example, to arrange one or more variable and/or constant domains into a suitable configuration, or to introduce codons, introduce cysteine residues, remove cysteine residues, modify, add or delete other amino acids, etc.

A functional fragment of an antibody can include any fragment that is capable of binding to an epitope with a detectable affinity, such as a Fab, Fab′, F(ab′)₂, Fd, Fv, dAb, single-chain variable (scFv), di-scFv, tri-scFv, microantibody, or minimal recognition unit consisting of the amino acid residues that mimic the hypervariable region of an antibody (e.g., an isolated complementarity determining region (CDR) such as a CDR3 peptide). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains can also be useful.

A functional fragment of an antibody will typically include at least one variable domain. The variable domain may be of any size or amino acid composition and will generally include at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, a functional fragment of an antibody may contain a monomeric V_{H} or V_{L} domain.

In particular configurations, a functional fragment of an antibody contains at least one variable domain covalently connected to at least one constant domain. Non-limiting, exemplary configurations of variable and constant domains that may be found within an antigen-binding fragment of an antibody of the present disclosure include: (i) V_{H}-C_{H1}; (ii) V_{H}-C_{H2}; (iii) V_{H}-C_{H3}; (iv) V_{H}-C_{H1}-C_{H2}; (v) V_{H}-C_{H1}-C_{H2}-C_{H3}; (vi) V_{H}-C_{H2}-C_{H3}; (vii) V_{H}-C_{L}; (viii) V_{L}-CHI; (ix) V_{L}-C_{H2}; (x) V_{L}-C_{H3}; (xi) V_{L}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly connected to one another or may be connected by a full or partial hinge or linker region. A hinge region may consist of at least 2 (e.g., at least 5, 10, 15, 20, 40, 60 or more) amino acids which result in a flexible or semi-flexible linkage between adjacent variable and/or constant domains in a single polypeptide molecule. Moreover, an antigen-binding fragment of an antibody may include a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full-length antibody molecules, functional fragments may be monospecific or multispecific (e.g., bispecific). A multispecific functional fragment of an antibody will typically include at least two different variable domains, wherein each variable domain is capable of binding to a separate antigen or to a different epitope on the same antigen. Any multispecific antibody format may be adapted for use in the context of an antigen-binding fragment of an antibody of the present disclosure using routine techniques available in the art.

An antibody of the present disclosure can include a pair of cysteines that are positioned to participate in a disulfide bridge or synthetic bridge (e.g., a two-carbon bridge). One or both cysteines of the pair may be in a reduced state due to the chemical environment (e.g., redox state, reduction state, or presence of reducing agents such as TCEP, DTBA or DTT) and/or due to the cysteines being separated due to denaturation or dissociation of the antibody. The cysteines of the pair may, nevertheless, be capable of forming a disulfide bridge or synthetic bridge when in the appropriate chemical environment and when the cysteines are in proximity to each other. Optionally, a pair of cysteines that participates in a disulfide bridge or synthetic bridge can include a sulfur moiety on a heavy chain of an antibody and a sulfur moiety on a light chain of an antibody. Alternatively or additionally, a pair of cysteines that participates in a disulfide bridge or synthetic bridge can include a sulfur moiety on a first heavy chain of an antibody and a sulfur moiety on a first light chain of an antibody.

An affinity moiety can be attached to an artificial polymer by a covalent bond. In some configurations, the attachment can consist essentially of a chain of covalent bonds such that separation of the affinity moiety from the artificial polymer would require breaking a covalent bond. Alternatively, an affinity moiety can be attached to an artificial polymer by at least one non-covalent bond. For example, the attachment can be broken by dissociating a non-covalent bond. Optionally, an affinity moiety can be attached to an artificial polymer by a combination of covalent and non-covalent bonds. Other moieties, such as a label moiety, can be attached to an artificial polymer using bonds exemplified herein for attaching an affinity moiety to an artificial polymer.

In some configurations, an affinity moiety can be attached to an artificial polymer via a thioether bond. For example, an affinity moiety can include a sulfur, such as a sulfur present in a cysteine moiety, and the thioether bond can include the sulfur and a carbon present in the artificial polymer. Optionally, an affinity moiety can be attached to an artificial polymer via two thioether bonds, each thioether bond occurring between a sulfur of the affinity moiety and a carbon of the artificial polymer. The two sulfurs can be present in cysteines of the affinity moiety. By way of more specific example, an affinity moiety can be attached to an artificial polymer via a moiety of Formula I:

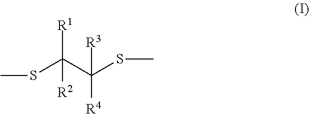

wherein S is a sulfur moiety of the affinity moiety, wherein at least one of R¹, R², R³, and R⁴ is attached to the artificial polymer, and wherein R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

Formula I can be a linear, non-cyclic structure. For example, an alkyl, alkenyl, alkynyl, aryl, heteroalkyl, heteroalkenyl, heteroalkynyl, heteroaryl or substituted variant of Formula I can form a linear structure. A pair of sulfurs, one or both of which can be present in a cysteine moiety, can be bridged via a linear ethyl moiety. For example, the linear ethyl moiety can be produced by reaction of a pair of cysteines with a propargyl moiety, whereby the sulfurs of the cysteines are bonded to the ethyl. The ethyl can be substituted with an amido moiety, for example, as a product of reaction of a pair of cysteines with the propargyl of a modifying reagent having an amido propargyl moiety.

Alternatively, Formula I can be a cyclic structure. For example, two or more of R¹, R², R³, and R⁴ of Formula I can occupy a ring structure. An example includes a pair of cysteines that are bridged via a dibenzocyclooctane moiety that bridges a pair of cysteines and further attaches the cysteines to another moiety. Optionally, cysteines can be linked via bonding of their sulfurs to the eight-membered ring of a dibenzocyclooctane moiety, the product of reaction with a linker having a dibenzocyclooctyne (DBCO) moiety. Exemplary species of Formula I and their synthesis are set forth in U.S. Pat. App. Ser. No. 63/394,907, which is incorporated herein by reference.

Antibodies are particularly well suited to attachments that include Formula I since antibodies typically have only a few cysteines, most of which are located at regions that are conserved across antibody structures. Taking a typical IgG antibody as an example, cysteines are located in the flexible hinge region (Fc) such that disulfide bonds occur between the two heavy chains heavy (HC1 and HC2). A typical IgG also has disulfide bonds between a cysteine in each heavy chain and a cysteine in each light chain (LC1 or LC2, respectively). The disulfides can be reduced to produce sulfurs that are sufficiently proximal to each other to form a structure satisfying Formula I. Accordingly, cysteine conjugation can provide a more targeted conjugation of an antibody compared to other conjugation chemistries in terms of the uniformity and reproducibility of conjugation products. Moreover, conjugation via Formula I maintains a bond between cysteines that are typically connected by a disulfide bridge. This is particularly advantageous for maintaining association between antibody subunits under reductive conditions or environments where an undesirable dissociation can occur. It will be understood that one or more cysteines can be introduced into an antibody or other polypeptide, for example, by well-known protein engineering methods, to produce a cysteine pair that can be modified as exemplified herein for native cysteine pairs in antibodies. Pairs of sulfurs that are sufficiently proximal in other affinity moieties, for example, nucleic acid aptamers or other non-proteinaceous moieties can also participate in a structure satisfying Formula I.

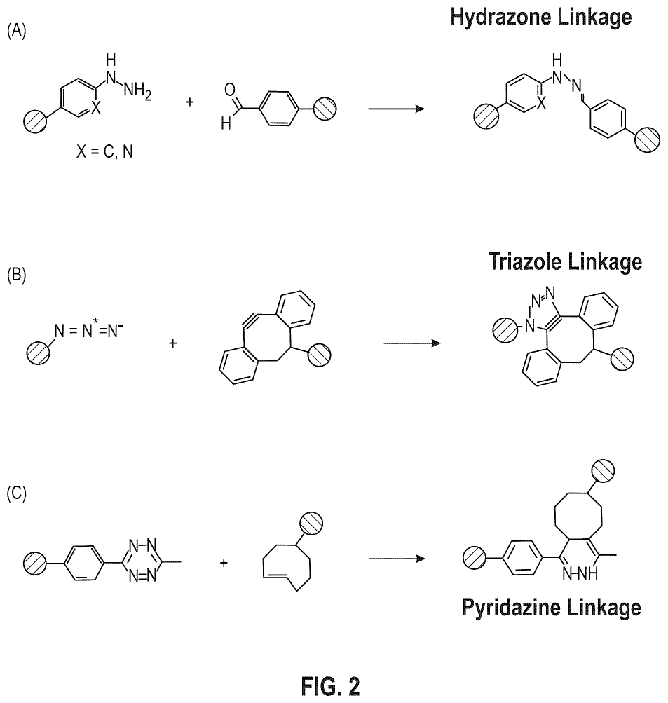

Any of a variety of chemistries can be used to covalently attach an attachment moiety or other functional moiety to an artificial polymer. Examples include click chemistries or chemistries set forth in U.S. Pat. No. 11,203,612; or U.S. Pat. App. Ser. No. 63/394,907, each of which is incorporated herein by reference. FIG. 2. shows useful click reagents and their reactions including: (A) conjugation of hydrazine and aldehyde via hydrazone linkage, (B) strain-promoted azide-alkyne click (SPAAC) chemistry, and (C) conjugation of methyl tetrazine (mTz) and trans-cyclooctene (TCO) via inverse electron demand Diels-Alder (IEDDA) reaction. See also the Examples section below. Another exemplary attachment utilizes the SpyTag/SpyCatcher system (See, Zakeri et al. Proceedings Nat'l Acad. Sciences USA. 109 (12): E690-7 (2012)). In this system, a 13 amino acid tag polypeptide (Spy Tag) forms a first coupling handle, with a 12.3 kDa protein (Spy-Catcher) forming the other coupling handle. The Spy Catcher can function by irreversibly bonding to a Spy Tag through an isopeptide bond. As will be appreciated, the Spy Tag or the Spy Catcher can be connected to an artificial polymer and the other member of the pair can be connected to an affinity moiety or other moiety that is attached to the artificial polymer.

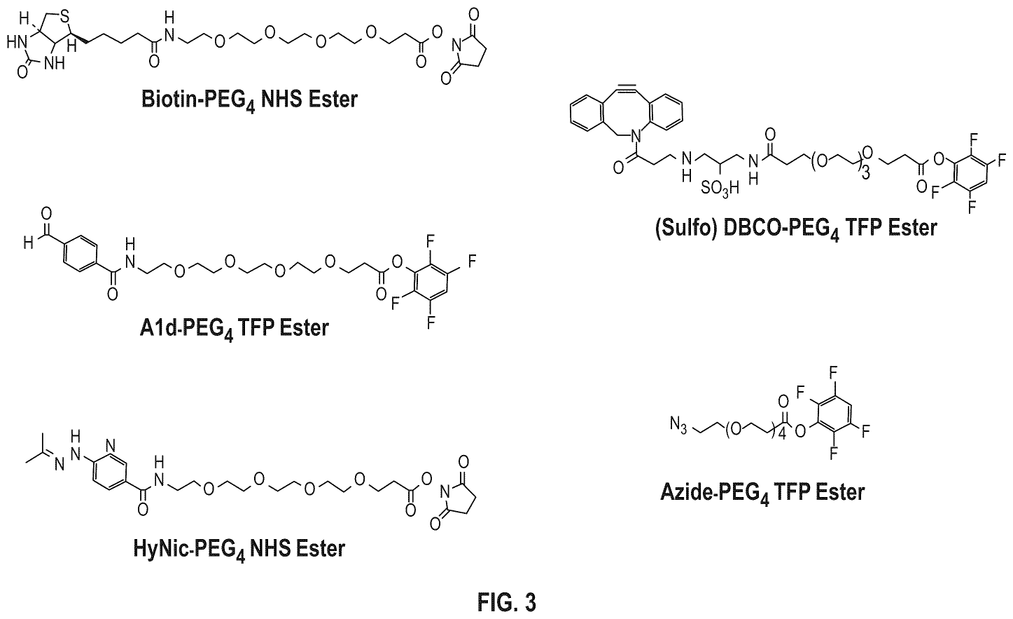

Any of a variety of non-covalent bonds can be used to attach an affinity moiety to an artificial polymer. Receptors and their ligands can be particularly useful. Examples include, but are not limited to, antibodies, antigens, (strept)avidin (or analogs thereof), biotin (or analogs thereof), Protein A, affibodies, affilins, affimers, affitins, alphabodies, anticalins, avimers, DARPins, monobodies, nanoCLAMPs, nucleic acids, peptide nucleic acids, polypeptides, nucleic acid aptamers, protein aptamers, lectins (or analogs thereof), carbohydrates or functional fragments thereof. Complementary nucleic acids can be used to non-covalently attach an affinity moiety to an artificial polymer. Useful nucleic acids can have complementary sequences that are at least 5, 10, 15, 20, 25, 30, 40, 50, 60, 70, 80, 90 or more nucleotides in length. Alternatively or additionally, nucleic acids can have complementary sequences that are at most 90, 80, 70, 60, 50, 40, 30, 25, 20, 15, 10, 5 or fewer nucleotides in length. Other reagents and techniques that can be used to non-covalently attach an affinity moiety or other functional moiety to an artificial polymer are set forth in U.S. Pat. No. 11,203,612; or U.S. Pat. App. Ser. No. 63/394,907, each of which is incorporated herein by reference. Receptors and ligands can be attached to artificial polymers using any of a variety of chemistries including, for example, those employing linkers shown in FIG. 3, those set forth in the Examples section below, and those set forth elsewhere herein.

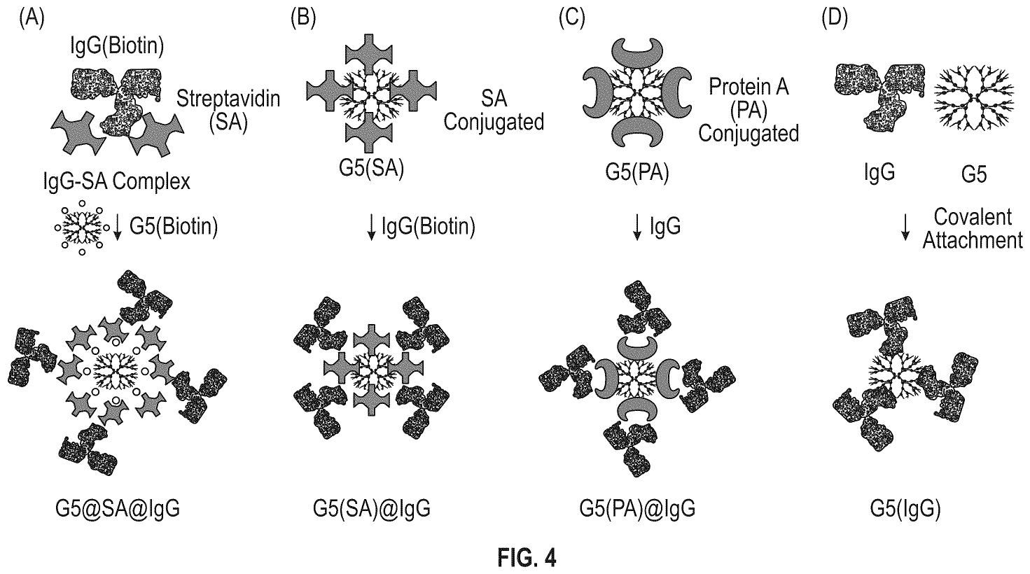

FIG. 4 shows exemplary affinity reagents having antibodies attached to artificial polymers, and reactions for making the affinity reagents. Reaction A shows formation of an affinity reagent (G5@SA @IgG) having a G5 artificial polymer covalently attached to biotin which is non-covalently bound to streptavidin which is covalently bound to an antibody. The G5@SA @IgG product is formed by reacting a biotinylated artificial polymer with antibodies that are functionalized with biotin for non-covalent bonding with streptavidin. The G5@SA@IgG product can include one to four antibodies and one to twenty-four labels. Reaction B shows formation of an affinity reagent (G5(SA)@IgG) having a G5 artificial polymer covalently attached to streptavidin which is non-covalently bound to biotin which is covalently attached to an antibody. The G5(SA)@IgG product is formed by reacting streptavidin-functionalized artificial polymer with biotinylated antibodies. The G5(SA)@IgG product can include one to four antibodies and one to twelve labels. Reaction C shows formation of an affinity reagent (G5(PA)@IgG) having a G5 artificial polymer covalently attached to Protein A which is non-covalently bound to an antibody. The G5(PA)@IgG product is formed by reacting Protein A-functionalized artificial polymer with antibodies. The G5(PA)@IgG product can include one to four antibodies and one to fourteen labels. Reaction D shows formation of an affinity reagent (G5(IgG)) having a G5 artificial polymer covalently attached to antibodies. The G5(IgG) product is formed by reacting a functionalized artificial polymer with antibody side chains. The G5(IgG) product can include one to three antibodies and one to nine labels. The reactions are exemplified and demonstrated in the Examples section below.

An affinity reagent of the present disclosure can include one or more affinity moieties. For example, an affinity reagent can include at least 1, 2, 3, 4, 5, 10 or more affinity moieties. Alternatively or additionally, an affinity reagent can include at most 10, 5, 4, 3, 2, or 1 affinity moieties. In some configurations, an artificial polymer can be attached to one or more affinity moieties. For example, an artificial polymer can be attached to at least 1, 2, 3, 4, 5, 10 or more affinity moieties. Alternatively or additionally, an artificial polymer can be attached to at most 10, 5, 4, 3, 2, or 1 affinity moiety.

Multiple affinity moieties that are present in an affinity reagent, or that are attached to a given artificial polymer, can have the same function or different functions compared to each other. For example, the multiple affinity moieties can have affinity for the same epitope or same set of epitopes. In some cases, the strength and/or specificity of the affinity can be substantially the same, for example, in cases where the affinity moieties have the same structure. In other cases, the strength and/or specificity of the affinity of two or more affinity moieties for a given epitope or set of epitopes can overlap despite some differences in the functional or structural characteristics of the two or more affinity moieties. In some configurations, multiple affinity moieties of a given affinity reagent can have affinity for different epitopes. This can be the case, whether the different epitopes are found in the same analyte (e.g., two different amino acid trimer epitopes present in a given polypeptide analyte) or in different analytes (e.g., a first trimer epitope being found in a first polypeptide that lacks a second trimer epitope, and a second trimer epitope being found in a second polypeptide that lacks the first trimer epitope).

Two or more affinity moieties can be attached to separate moieties of an artificial polymer. For example, the two or more affinity moieties can be attached to separate branches of the artificial polymer or to separate monomers of an artificial polymer. In other cases, two or more affinity moieties can be attached to the same moiety of an artificial polymer or to the same branch of an artificial polymer.

A wide variety of labels can be used in a composition or method set forth herein including, for example, optically detectable labels, such as luminophores (e.g., fluorophores), enzymes (e.g., enzymes which catalyze reactions with colored reagents or products), electrochemical labels (e.g., highly charged moieties). Magnetic contrast imaging moieties can be used such as gadolinium-diethylenetriaminepentacetate (Gd-DTPA), gadolinium-dodecane tetraacetic acid (Gd-DOTA) or others used for magnetic resonance techniques. Moieties that are detected through subsequent processing, such as nucleic acid barcode labels, can also be useful. Nucleic acids can be detected or identified via nucleic acid amplification, sequencing or hybridization assays.

Any of a variety of luminophores may be used herein. In some cases, the luminophore may be a small molecule. In some cases, the luminophore may be a polypeptide. Luminophores may include labels that emit in the ultraviolet, visible, or infrared region of the spectrum. In some cases, the luminophore may be selected from the group consisting of FITC, Alexa Fluor® 350, Alexa Fluor® 405, Alexa Fluor® 488, Alexa Fluor® 532, Alexa Fluor® 546, Alexa Fluor® 555, Alexa Fluor® 568, Alexa Fluor® 594, Alexa Fluor® 647, Alexa Fluor® 680, Alexa Fluor® 750, Pacific Blue, Coumarin, BODIPY FL, Pacific Green, Oregon Green, Cy3, Cy5, Pacific Orange, TRITC, Texas Red, R-Phycoerythrin, Allophcocyanin (APC). In some cases, the label may be an Atto dye, for example Atto 390, Atto 425, Atto 430, Atto 465, Atto 488, Atto 490, Atto 495, Atto 514, Atto 520, Atto 532, Atto 540, Atto 550, Atto 565, Atto 580, Atto 590, Atto 594, Atto 610, Atto 611, Atto 612, Atto 620, Atto 633, Atto 635, Atto 647, Atto 655, Atto 680, Atto 700, Atto 725, Atto 740, Atto MB2, Atto Oxal2, Atto Rhol01, Atto Rhol2, Atto Rhol3, Atto Rho14, Atto Rho3B, Atto Rho6G, or Atto Thiol2. In some cases, the luminophore may be a fluorescent protein such as green fluorescent protein (GFP), cyan fluorescent protein (CFP), red fluorescent protein (RFP), blue fluorescent protein (BFP), orange fluorescent protein (OFP), and yellow fluorescent protein (YFP). A wide range of effective luminophores are commercially available, for example, from the Molecular Probes division of ThermoFisher Scientific and/or generally described in the Molecular Probes Handbook (11th Edition) which is hereby incorporated by reference. Label components may also include intercalation dyes, such as ethidium bromide, propidium bromide, crystal violet, 4′,6-diamidino-2-phenylindole (DAPI), 7-aminoactinomycin D (7-AAD), Hoescht 33258, Hoescht 33342, Hoescht 34580, YOYO-1, DiYO-1, TOTO-1, DiTO-1, or combinations thereof.

An affinity reagent of the present disclosure can include one or more label moieties. For example, an affinity reagent can include at least 1, 2, 3, 4, 5, 10, 15, 20 or more label moieties. Alternatively or additionally, an affinity reagent can include at most 20, 15, 10, 5, 4, 3, 2, or 1 label moieties. In some configurations, an artificial polymer can be attached to one or more label moieties. For example, an artificial polymer can be attached at least 1, 2, 3, 4, 5, 10, 15, 20 or more label moieties. Alternatively or additionally, an artificial polymer can be attached to at most 20, 15, 10, 5, 4, 3, 2, or 1 label moieties.

Multiple label moieties that are present in an affinity reagent, or that are attached to a given artificial polymer, can have the same structure or they can differ structurally from each other. Optionally, multiple label moieties can have the same detectable characteristics or they can have characteristics that are distinguishable from each other. For example, two or more optical labels can differ in terms of luminescence lifetime, luminescence polarity, extinction coefficient, quantum yield of luminescence, spectral region for absorbance, spectral region for excitation or spectral region for emission. Alternatively, two or more optical labels can be indistinguishable in terms of luminescence lifetime, luminescence polarity, extinction coefficient, quantum yield of luminescence, spectral region for absorbance, spectral region for excitation or spectral region for emission. This can result from the two or more optical labels having the same structure, but in some cases two or more labels can have the same or overlapping detection properties despite having different structures.

One or more label moieties can be attached to an artificial polymer using bonds (e.g., covalent bonds and/or non-covalent bonds) set forth herein, for example, in the context of attaching affinity moieties to artificial polymers. Two or more labels can be attached to separate moieties of an artificial polymer. For example, the two or more labels can be attached to separate branches of the artificial polymer or separate monomers of the artificial polymer. In other cases, two or more labels can be attached to the same moiety of an artificial polymer or to the same branch of an artificial polymer.

An affinity moiety can be attached to an artificial polymer using the same type of bond that attaches a label moiety to the artificial polymer. For example, the affinity reagent can be synthesized using the same chemistry for attaching the label moiety and the affinity moiety to the artificial polymer. Alternatively, an affinity moiety can be attached to an artificial polymer using a different type of bond compared to the bond that attaches a label moiety to the artificial polymer. For example, the affinity reagent can be synthesized using different chemistry for attaching the affinity moiety to an artificial polymer compared to the chemistry used for attaching the label moiety to the artificial polymer. Different chemistries that are used to attach different functional moieties to an artificial polymer can optionally be orthogonal chemistries. However, orthogonality is not necessary for all configurations that utilize different chemistries.

Affinity moieties, label moieties and other moieties can be attached in various configurations to an affinity reagent set forth herein. For example, a label can be attached to an affinity moiety. As such, a label can be attached to an artificial polymer via an affinity moiety. Conversely, an affinity moiety can be attached to an artificial polymer via a label. A plurality of labels can be attached to a single affinity moiety or a plurality of affinity moieties can be attached to a single label.

An affinity moiety can be attached to a first branch of an artificial polymer and a label moiety can be attached to a second branch of the artificial polymer. Alternatively or additionally, an affinity moiety and a label moiety can be attached to the same branch of an artificial polymer. An affinity moiety can be attached to a first moiety of an artificial polymer and a label moiety can be attached to a second moiety of the artificial polymer. Alternatively or additionally, an affinity moiety and a label moiety can be attached to the same moiety of an artificial polymer.

The present disclosure further provides a complex including an affinity reagent non-covalently bound to an analyte, wherein the affinity reagent includes (a) an artificial polymer having a branched chain; (b) an affinity moiety attached to the artificial polymer; and (c) a label attached to the artificial polymer, and wherein the analyte is non-covalently bound to the affinity reagent via the affinity moiety. The label is optional. Accordingly, a complex can include an affinity reagent non-covalently bound to an analyte, wherein the affinity reagent includes (a) an artificial polymer having a branched chain; and (b) an affinity moiety attached to the artificial polymer. The artificial polymer can also be removed or replaced as an option.

Any of a variety of analytes can be present in a complex set forth herein. A particularly useful analyte is a polypeptide. Other analytes that can be used include, but are not limited to, a tissue, cell, organelle, virus, nucleic acid (e.g., DNA or RNA), carbohydrate (e.g., monosaccharide, oligosaccharide or polysaccharide), vitamin, enzyme cofactor, hormone, small molecule such as a candidate therapeutic agent, metabolite, nucleotide, nucleoside, amino acid, sugar, lipid, or the like. These and other analytes known in the art can be used in combination with compositions and methods set forth herein.

An analyte that is used in a composition or method set forth herein can be in a fluid (i.e. a fluid-phase analyte). As such, an analyte can be diffusible in fluid. Alternatively, an analyte can be immobilized on a solid support (i.e. a solid-phase analyte). In some configurations, an analyte can be attached to a particle, such as a structured nucleic acid particle. The particle-attached analyte can be fluid-phase or solid-phase. For example, the analyte can be attached to a solid support via the particle.

Nucleic acid origami provides a particularly useful material for a particle. Accordingly, a particle can include one or more nucleic acids having tertiary or quaternary structures such as spheres, cages, tubules, boxes, triangles, icosahedrons, tiles, blocks, trees, pyramids, wheels or combinations thereof. Examples of such structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. No. 8,501,923 or 9,340,416, each of which is incorporated herein by reference. In some configurations, a nucleic acid origami may include a scaffold and a plurality of staples. The scaffold can be configured as a single, continuous strand of nucleic acid, and the staples can be formed by oligonucleotides that hybridize, in whole or in part, with the scaffold nucleic acid. A particle including one or more nucleic acids (e.g., as found in origami or nanoball structures) may include regions of single-stranded nucleic acid, regions of double-stranded nucleic acid, or combinations thereof.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold nucleic acid can be linear (i.e., having a 3′ end and 5′ end) or circular (i.e. closed such that the scaffold lacks a 3′ end and 5′ end). A nucleic acid scaffold can be derived from a natural source, such as a viral genome or a bacterial plasmid. For example, a nucleic acid scaffold can include a single strand of an M13 viral genome. In other configurations, a nucleic acid scaffold may be synthetic, for example, having a non-naturally occurring sequence in full or in part. A scaffold nucleic acid can be single stranded but for a plurality of oligonucleotides hybridized thereto or short regions of internal complementarity. The size of a nucleic acid scaffold may vary to accommodate different uses. For example, a nucleic acid scaffold may include at least about 100, 500, 1000, 2500, 5000, 10000, 50000 or more nucleotides. Alternatively or additionally, a nucleic acid scaffold may include at most about 50000, 10000, 5000, 2500, 1000, 500, 100 or fewer nucleotides.

A nucleic acid origami can include a plurality of oligonucleotides that are hybridized to a scaffold nucleic acid. A first region of an oligonucleotide sequence can be hybridized to a scaffold nucleic acid while a second region is not hybridized to the scaffold. The second region can be in a single stranded state or, alternatively, can participate in a hairpin or other self-annealed structure in the oligonucleotide. In some cases, the second region of the oligonucleotide can hybridize to a complementary oligonucleotide to form a double-stranded region. An oligonucleotide can include two sequence regions that are hybridized to a scaffold nucleic acid, for example, to function as a ‘staple’ that restrains the structure of the scaffold. For example, a single oligonucleotide can hybridize to two regions of a scaffold that are separated from each other in the primary sequence of the scaffold. As such, the oligonucleotide can function to retain those two regions of the scaffold in proximity to each other or to otherwise constrain the scaffold to a desired conformation. Two sequence regions of an oligonucleotide staple can be adjacent to each other in the oligonucleotide sequence or separated by a third region that does not hybridize to the scaffold. One or more regions of an oligonucleotide that hybridize to a scaffold nucleic acid can be located at or near the 5′ end of the oligonucleotide, at or near the 3′ end of the oligonucleotide, or in a region of the oligonucleotide that is between the end regions. Oligonucleotides can be configured to hybridize with a nucleic acid scaffold, another oligonucleotide, a staple oligonucleotide, or a combination thereof. The oligonucleotides can be linear (i.e. having a 3′ end and a 5′ end) or closed (i.e. circular, lacking both 3′ and 5′ ends).

An oligonucleotide that is included in a nucleic acid origami can have any of a variety of lengths. An oligonucleotide may have a length of at least about 10, 25, 50, 100, 250, 500, or more nucleotides. Alternatively or additionally, an oligonucleotide may have a length of no more than about 500, 250, 100, 50, 25, 10, or fewer nucleotides. An oligonucleotide in a nucleic acid origami may hybridize with another oligonucleotide or a scaffold strand forming a particular number of base pairs. An oligonucleotide may form a hybridization region of at least about 5, 6, 7, 8, 9, 10, 15, 20, 25, 50 or more consecutive or total base pairs. Alternatively or additionally, an oligonucleotide may form a hybridization region of no more than about 50, 25, 20, 15, 10, 9, 8, 7, 6, 5, or fewer consecutive or total base pairs.

A molecule or analyte set forth herein can be attached to nucleic acid origami via a scaffold component or oligonucleotide component of the origami structure. For example, the scaffold or oligonucleotide can include a nucleotide analog that attaches covalently or non-covalently to the antibody conjugate, label moiety or other moiety. Examples of structured nucleic acid particles that include nucleic acid origami are set forth, for example, in U.S. Pat. No. 11,203,612; U.S. Pat. App. Pub. No. 2022/0162684 A1 or U.S. patent application Ser. No. 17/692,035, each of which is incorporated herein by reference.

A particle need not be composed primarily of nucleic acid and, in some cases, may be devoid of nucleic acids. For example, an analyte can be attached to an artificial polymer that is configured to form a particle. In other examples, a particle can be composed of a solid support material, such as a silicon or silica nanoparticle, a carbon nanoparticle, a cellulose nanobead, a PEG nanobead, upconversion nanocrystal, or a quantum dot. A particle to which an analyte is attached can have a size or other characteristic exemplified for particles composed of artificial polymers.

An analyte can be located at an address of an array. As such, an affinity reagent set forth herein can be located at an address of an array due to forming a complex with an analyte located at the address. In some configurations, the array is configured for single-molecule resolution. For example, individual addresses of the array can each be attached to one, and only one, analyte. Moreover, the single analyte can be attached to a single affinity reagent. As such, an address of an array can be attached to one, and only one, affinity reagent. Alternatively, individual addresses of the array can each be attached to an ensemble of analytes. Moreover, the ensemble of analytes can be attached to a plurality of affinity reagents. As such, an address of an array can be attached to a plurality of affinity reagents.

The addresses of an array can optionally be optically observable and, in some configurations, adjacent addresses can be optically distinguishable when detected. Addresses of an array are typically discrete. The discrete addresses can be contiguous, or they can have interstitial spaces between each other. An array useful herein can have, for example, addresses that are separated by less than 100 microns, 10 microns, 1 micron, 500 nm, 100 nm, 10 nm or less. Alternatively or additionally, an array can have addresses that are separated by at least 10 nm, 100 nm, 500 nm, 1 micron, 5 microns, 10 microns, 50 microns, 100 microns or more. The addresses can each have an area of less than 1 square millimeter, 500 square microns, 100 square microns, 25 square microns, 1 square micron or less. An array can include at least about 1×10⁴, 1×10⁵, 1×10⁶, 1×10⁸, 1×10¹⁰, 1×10¹², or more addresses.

The present disclosure provides a method of making an affinity reagent. The method can include steps of (a) providing an artificial polymer having a branched chain, wherein at least one chain of the artificial polymer has an alkyne moiety; and (b) reacting a cysteine of an antibody with the alkyne moiety, thereby covalently attaching the antibody to the artificial polymer.

Step (b) of the above method can be configured to react a single cysteine of the antibody with the alkyne moiety. As such, the antibody can be attached to the artificial polymer via a single cysteine. Alternatively, step (b) of the above method can be configured to react two cysteines of the antibody with the alkyne moiety, thereby attaching the antibody to the artificial polymer via two cysteines.

In particular configurations, a pair of cysteines of an antibody, or other proteinaceous moiety, can be reacted with an alkyne moiety of an artificial polymer under irradiation with light to drive formation of a two-carbon bridge between the cysteines, such as the bridge of Formula I. An alkyne moiety can react with a pair of sulfurs (e.g., the sulfurs present in a pair of cysteines) to form a moiety of Formula I:

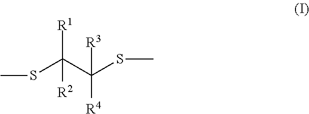

wherein S is sulfur, wherein at least one of R¹, R², R³, and R⁴ is attached to the artificial polymer, and wherein R¹, R², R³, and R⁴ are each independently selected from the group consisting of hydrogen, halo, alkyl, alkenyl, alkynyl, aryl, heteroalkyl, heteroalkenyl, heteroalkynyl, heteroaryl, and optionally substituted variants thereof.

Photocatalysts such as azo-based compounds can be used to generate radical species for an irradiation step of a method set forth herein. Exemplary photocatalysts include, but are not limited to, 2,2-azobis(isobutyronitrile) (AIBN), azobis(2-methylpropionamidine) dihydrochloride (AAPH), benzophenone, 2,2-dimethoxy-2-phenylacetophenone, and lithium phenyl-2,4,6-trimethylbenzoylphosphinate (LAP). Without intending to be limited by mechanism, the reaction can be, at least in some configurations of the methods set forth herein, a photo thiol-yne reaction. Light can be provided at a wavelength or wavelength range that is compatible with the reaction components. For example, the light can be in the UV range of the spectrum (10 nm to 400 nm). In some cases, it may be desirable to use light in different sub-regions of the UV including, for example, the long wavelength ultraviolet (UVA) range (315 nm to 400 nm), medium wavelength ultraviolet (UVB) range (280 nm to 315 nm), or short wavelength ultraviolet (UVC) range (200 nm to 280 nm). Light in the longer wavelengths, for example, in the visible (VIS) or near infrared (NIR) regions of the spectrum can be used, for example, to improve light penetration and absorption by photocatalysts such as azobis(2-methylpropionamidine) dihydrochloride (AAPH), or to minimize damage to antibodies or other components that are present in the reaction. Alternatively, light can be delivered at the lower wavelengths or middle wavelengths of the range to achieve higher reaction yield. Although delivery of light at wavelengths below 400 nm, 315 nm or 280 nm can be desirable, it may also be useful to deliver light at higher wavelengths, for example, in the visible region of the spectrum such as wavelengths at or below the red, orange, yellow, green, blue, or violet regions of the visible spectrum. This can be achieved using a mechanism of two-photon absorption or by adding upconversion nanocrystals which are emitting UV-VIS bands of light upon light excitation at a near infrared region (e.g., 808 nm or 980 nm). Light that is delivered in a reaction between cysteines and an alkyne moiety can exclude wavelengths in a particular range of the spectrum including, but not limited to, one or more of the ranges set forth herein.

Light can be delivered for a time period that is suitable to achieve a desired yield of antibody attachment to an artificial polymer. For example, light can be delivered for at least 1, 2, 5, 10, 15, or 30 minute(s). Alternatively or additionally, it may be desirable to deliver light for at most 30, 15, 10, 5, 2 or 1 minute(s). The radiant flux of the light can be, for example, at least 8 watts, 10 watts, 100 watts, 200 watts, 300 watts or more. In some cases, lower wattage can be used.

A method of the present disclosure can be configured to react an alkyne moiety with a sulfur moiety on a heavy chain of an antibody and a sulfur moiety on a light chain of the antibody, thereby attaching the antibody to an artificial polymer (or other molecule) via a synthetic crosslink between the heavy chain and the light chain. As an alternative or addition to modifying at least one cysteine pair to form synthetic crosslink(s) between heavy and light chain(s), a method of the present disclosure can be configured to react an alkyne moiety with a sulfur moiety on a first heavy chain of the antibody and a sulfur moiety on a second heavy chain of the antibody, thereby attaching the antibody to an artificial polymer (or other molecule) via a synthetic crosslink between the first and second heavy chains.

In many cases, an antibody that is to be reacted in a method set forth herein, will contain a pair of cysteines that are bridged by a disulfide. The bridged cysteines can be reduced prior to being reacted with an alkyne moiety or other modifying reagent. The antibody can be contacted with any of a variety of reducing agents including, for example, dithiothreitol (DTT), 2-mercaptoethanol (2-ME), tris(2-carboxyethyl)phosphine (TCEP), tris(3-hydroxypropyl)phosphine) (THPP), 2-aminoethanethiol, glutathione, and 1,4-dithio-2-butylamine (DTBA).

In some configurations of the methods set forth herein, pairs of cysteines in different regions of an antibody are selectively reduced. Selective reduction of cysteines in an antibody followed by reaction of the selectively reduced antibody with an alkyne moiety can yield an antibody conjugate that contains a pair of cysteines that is selectively modified to contain a two-carbon bridge, while at least one other pair of cysteines in the antibody is not modified to have a two-carbon bridge. Optionally, a pair of cysteines that forms a disulfide bridge between a heavy chain and light chain can be selectively reduced while other pairs of cysteines are in an oxidized state. For example, disulfides between heavy and light chains can be preferentially reduced using less than 4 molar equivalents of a strong reducing agent such as DTT or TCEP compared to the quantity of antibody in the reaction (see Sun et al. Bioconjug Chem. 16(5): 1282-1290 (2005), which is incorporated herein by reference). In an alternative option, a pair of cysteines that forms a disulfide bridge between heavy chains can be selectively reduced while other pairs of cysteines remain in an oxidized state. For example, disulfides between heavy chains can be preferentially reduced by fully reducing all cysteine pairs in an antibody and then selectively re-oxidizing cysteines that form disulfides between heavy and light chains of the antibody. The selective reoxidation can be achieved by treatment of the fully reduced antibody with 2 to 3 molar equivalents of DTNB or 4,4′-dipyridyldithiol compared to the quantity of reduced antibody (see Sun et al. Bioconjug Chem. 16(5): 1282-1290 (2005), which is incorporated herein by reference).

An antibody or other polypeptide can be genetically engineered to add or remove cysteines prior to being modified in a method set forth herein. For example, one or more cysteines can be removed to preclude unwanted modification of the antibody or other polypeptide at the site of the removed cysteine(s). For example, an antibody can be engineered to omit at least 1, 2, 3 or 4 cysteines that would otherwise form a disulfide bridge between two heavy chains. Alternatively or additionally, an antibody can be engineered to omit at least 1, 2, 3 or 4 cysteines that would otherwise form a disulfide bridge between a heavy chain and light chain. Whether or not any cysteines are removed from an antibody, the antibody can be engineered to add one or more cysteines at one or more respective positions where cysteine(s) are not typically found in nature. An added cysteine can be modified to form an attachment to another moiety such as an artificial polymer. Optionally, the added cysteine can participate in a two-carbon bridge with another cysteine in the structure of the engineered antibody. In some configurations, a cysteine that has been added to an engineered antibody or other polypeptide is modified in a method of the present disclosure to form a two-carbon bridge (e.g., a bridge having a structure of Formula I) with a native cysteine in the engineered antibody or polypeptide. The reaction can be configured to attach the antibody or other polypeptide to an artificial polymer (or other alkyne-containing molecule) via a two-carbon bridge. Alternatively, a cysteine that has been added to an engineered antibody or other polypeptide can be modified in a method of the present disclosure to form a two-carbon bridge with another added cysteine in the engineered antibody or polypeptide. The reaction can be configured to attach the antibody or other polypeptide to an artificial polymer via a two-carbon bridge. One or more cysteines can be added to an engineered antibody to facilitate attachment of an artificial polymer (or other alkyne-containing molecule) to the engineered antibody via a two-carbon bridge (e.g., a bridge having a structure of Formula I) between two heavy chains, between a heavy chain and a light chain, within the same heavy chain or within the same light chain.

The use of light to drive reaction of alkyne moiety with a pair of sulfurs can be particularly useful when the alkyne moiety is not present in a strained ring. For example, the alkyne moiety can be in a linear hydrocarbon or in a ring having at least 6, 7, 8 or more members. However, a method set forth herein can include delivery of light to a reaction that occurs between a pair of sulfurs and an alkyne moiety that is present in a strained ring. Attachment of an antibody to an artificial polymer via a two-carbon bridge between cysteines of the antibody, can employ an artificial polymer having a cyclooctyne, cycloheptyne, cyclohexyne or cyclopentyne moiety. A dibenzocyclooctyne moiety can be particularly useful. Other cyclooctyne moieties can be particularly useful such as those derived from 3,3-difluoro-substituted cyclo-1-octyne and cyclopropane-fused bicyclo[6.1.0]nonyne.

In some configurations of the methods set forth herein, light is not delivered to a reaction between cysteines and an alkyne moiety. For example, the reaction can be carried out in absence of light or under standard laboratory lighting that is not enriched for light having wavelengths at or below the UV range or other spectral range set forth herein. Reactions that are not activated by light can be carried out using an alkyne that is activated by other mechanisms, such as via ring strain. In some cases, an alkyne can be reacted with sulfhydryls at elevated temperatures such that radicals are formed thermally and stimulate formation of a sulfur cross link as set forth herein. Elevated temperatures can include temperatures above, 40° C., 45° C., 50° C., 60° C., 70° C. or higher. Alternatively or additionally, the temperature can be maintained below 70° C., 60° C., 50° C., 45° C., 40° C., or lower. For example, the temperature can be capped to maintain structural integrity and/or activity of an antibody that is to be crosslinked or otherwise modified in a method set forth herein.

It will be understood that reactions exemplified herein for attaching an antibody to an artificial polymer can be extended to other molecules having pairs of proximal sulfurs and alkynes respectively. For example, other polypeptides may have pairs of cysteines that are configured to participate in reaction with an alkyne. Moreover, antibodies or other polypeptides can be engineered to include pairs of proximal cysteines. A sulfur or pair of sulfurs that reacts with an alkyne need not be present in a cysteine, for example, being provided by a chemical modification of a polypeptide (e.g., an antibody) or otherwise being present in a reactant that is to be attached to an artificial polymer having an alkyne moiety. For example, a nucleic acid aptamer or other affinity reagent can include two sulfur moieties that are configured to react with an alkyne moiety of an artificial polymer.

A method of making an affinity reagent can include steps of (a) providing an artificial polymer having a branched chain, wherein at least one chain of the artificial polymer includes a first member of a receptor-ligand pair; (b) providing an affinity component having a second member of the receptor-ligand pair; and (c) binding the first and second members of the receptor-ligand pair, whereby the affinity component forms an affinity moiety of the artificial polymer.

Members of a receptor-ligand pair can be attached to an artificial polymer or affinity component using chemistries set forth herein or known in the art. For example, a covalent attachment method can be used such as a click chemistry reaction. Several reactions are shown in FIG. 4 and/or demonstrated in the Examples section below. In some cases, a member of a receptor ligand pair can be a moiety of an affinity component. For example, Protein A can be attached to an artificial polymer and can non-covalently bind to the Fc region of an antibody. In another example, an affinity reagent can include a secondary antibody that is attached to an artificial polymer and the secondary antibody can non-covalently bind to an epitope in a primary antibody, wherein the primary antibody functions as an affinity moiety of the affinity reagent.

A method of the present disclosure can be configured to attach two or more different moieties to an artificial polymer. The two or more moieties can be attached using orthogonal reactions, respectively. As such, a reaction for attaching a first moiety to an artificial polymer can occur without interfering with a reaction for attaching a second moiety to the artificial polymer. For example, an alkyne moiety of an artificial polymer can be reacted with a cysteine, or pair of cysteines, in a first reaction and a label can be attached to the artificial polymer using a second reaction that is orthogonal to the first reaction. In one option, the second reaction can include reacting an azide moiety on the artificial polymer with a strained alkane or strained alkyne in a click reaction. The first reaction can be carried out prior to the second reaction; however, the order can be reversed if desired.

in some configurations, a method of making an affinity reagent can include steps of (a) providing an artificial polymer having a branched chain, wherein at least one chain of the artificial polymer has an alkyne moiety; (b) reacting a cysteine of an antibody with the alkyne moiety, thereby covalently attaching the antibody to the artificial polymer, and (c) reacting a second moiety of the artificial polymer with a label, thereby attaching the label to the artificial polymer. Step (b) can be performed prior to step (c) or, alternatively, step (b) can be performed after step (c). If desired, steps (b) and (c) can be carried out in parallel.

In another configuration, a method of making an affinity reagent can include steps of (a) providing an artificial polymer having a branched chain, wherein at least one chain of the artificial polymer includes a first member of a receptor-ligand pair; (b) providing an affinity component having a second member of the receptor-ligand pair; (c) binding the first and second members of the receptor-ligand pair, whereby the affinity component forms an affinity moiety of the artificial polymer, and (d) reacting a second moiety of the artificial polymer with a label, thereby attaching the label to the artificial polymer. Step (c) can be performed prior to step (d) or, alternatively, step (c) can be performed after step (d). If desired, steps (c) and (d) can be carried out in parallel.

An artificial polymer can be attached to label moiety via chemical reaction of a reactive moiety on the polymer with a reactive moiety on the label. Any of a variety of chemical reactions can be used, including but not limited to, a click reaction or a reaction set forth herein in the context of attaching an affinity moiety to an artificial polymer. For example, conjugation of a functional moiety, such as a label moiety, to an artificial polymer or other moiety may proceed via an amide formation reaction, reductive amination reaction, N-terminal modification, thiol Michael addition reaction, disulfide formation reaction, copper(I)-catalyzed alkyne-azide cycloaddition (CuAAC) reaction, strain-promoted alkyne-azide cycloaddition reaction (SPAAC), Strain-promoted alkyne-nitrone cycloaddition (SPANC), inverse electron-demand Diels-Alder (IEDDA) reaction, oxime/hydrazone formation reaction, free-radical polymerization reaction, or a combination thereof.

Moieties that participate in cycloaddition reactions may be utilized as attachment moieties. In cycloaddition reactions, two or more unsaturated moieties form a cyclic product with a reduction in the degree of unsaturation, these reaction partners are typically absent from natural systems, and so the use of cycloadditions for conjugation utilizes the introduction of unnatural functionality within a coupling partner. Exemplary moieties and their attachment reactions include:

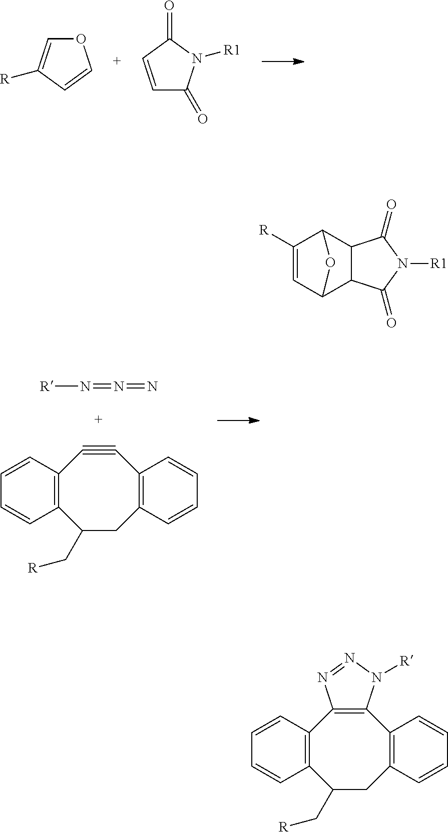

In some cases, reactive moieties that participate in Copper-Catalyzed Azide-Alkyne Cycloadditions (CuAAC) may be utilized to attach functional moieties to an artificial polymer. Optionally, reactive moieties that participate in Strain-Promoted Azide-Alkyne Cycloadditions (SPAAC) may be utilized to attach functional moieties to artificial polymer moieties or other moieties. One of an azide or alkyne that is connected to a functional moiety can be reacted with an artificial polymer having an alkyne or azide, respectively. A CuAAC or SPAAC reaction can be performed to produce a triazole attachment of an antibody or other functional moiety to an artificial polymer moiety or other moiety.

Moieties that participate in inverse-electron demand Diels-Alder (IEDDA) reactions may be utilized to attach artificial polymers to various functional moieties. One of a 1,2,4,5-tetrazine moiety, strained alkene moiety or strained alkyne can be used in an IEDDA reaction. Exemplary moieties include, but are not limited to, trans-cyclooctenes, functionalized norbornene derivatives, triazines, or spirohexene. In some cases, a maleimide or furan can be used in a hetero-Diels-Alder cycloaddition between a maleimide and furan. In some cases, a Diels-Alder reaction can achieve covalent coupling of a diene moiety with an alkene moiety to form a six-membered ring complex for attachment.

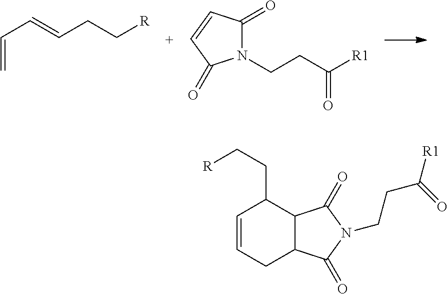

Accordingly, a diene moiety, alkene moiety, or hydrazone can be utilized in an attachment step set forth herein. For example, attachment can be achieved via difunctional cross-linking.

Any of a variety of non-covalent bonds can be used to attach a label moiety to an artificial polymer including, but not limited to those set forth herein in the context of attaching an affinity moiety to an artificial polymer. Other reagents and techniques that can be used to non-covalently attach a label moiety or other functional moiety to an artificial polymer are set forth in U.S. Pat. No. 11,203,612; or U.S. Pat. App. Ser. No. 63/394,907, each of which is incorporated herein by reference.

The present disclosure further provides a method of detecting an analyte (e.g., a polypeptide). The method can include steps of (a) providing an affinity reagent having an artificial polymer comprising a branched chain that is attached to an affinity moiety and a label; (b) binding the affinity moiety of the affinity reagent to an analyte; and (c) detecting the label, thereby detecting binding of the affinity reagent to the analyte.

Any of a variety of assay formats can be used to detect an analyte. Several methods will be exemplified below in the context of detecting polypeptides but can readily be extended to other analytes by modifications that will be apparent to those skilled in the art.

A polypeptide can be detected using one or more affinity reagents having binding affinity for the polypeptide. The affinity reagent and the polypeptide can bind each other to form a complex and the complex can be detected during or after formation. The complex can be detected directly, for example, due to a label that is present on the affinity reagent or polypeptide. In some configurations, the complex need not be directly detected, for example, in formats where the complex is formed and then the affinity reagent, polypeptide, or a label component that was present in the complex is subsequently detected.

Many polypeptide assays, such as enzyme linked immunosorbent assay (ELISA), achieve high-confidence characterization of one or more polypeptides in a sample by exploiting high specificity binding of affinity reagents to the polypeptide(s) and detecting the binding event while ignoring all other polypeptides in the sample. Binding assays can be carried out by detecting affinity reagents and/or polypeptides that are immobilized in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FEXMAP 3D®.

Exemplary assay formats that can be performed at a variety of plexity scales up to and including proteome scale are set forth in U.S. Pat. No. 10,473,654 or U.S. Pat. App. Pub. Nos. 2020/0318101 A1 or 2020/0286584 A1; U.S. patent application Ser. No. 18/045,036, or Egertson et al., BioRxiv (2021), DOI: 10.1101/2021.10.11.463967, each of which is incorporated herein by reference. A plurality of polypeptides can be assayed for binding to affinity reagents, for example, on single-molecule resolved polypeptide arrays. Polypeptides can be in a denatured state or native state when manipulated or detected in a method set forth herein.

Turning to the example of an array-based assay configuration, the identity of an extant polypeptide at any given address is typically not known prior to performing the assay. The assay can be used to identify extant polypeptides at one or more addresses in the array. A plurality of affinity reagents, optionally labeled (e.g., with fluorophores), can be contacted with the array, and the presence of affinity reagents can be detected at individual addresses to determine binding outcomes. A plurality of different affinity reagents can be delivered to the array and detected serially, such that each cycle detects binding outcomes for a given type of affinity reagent (e.g., a type of affinity reagent having affinity for a particular epitope) at each address. The assay can include a step of dissociating affinity reagents from the array after detecting the binding outcomes, such that the next affinity reagent can be delivered to the flow cell and detected. In some configurations, a plurality of different affinity reagents can be detected in parallel, for example, when different affinity reagents are distinguishably labeled.

In particular configurations, a method set forth herein can be used to identify a number of different extant polypeptides that exceeds the number of affinity reagents used. For example, the number of different polypeptide species identified can be at least 5×, 10×, 25×, 50×, 100× or more than the number of affinity reagents used. This can be achieved, for example, by (1) using promiscuous affinity reagents that bind to multiple different candidate polypeptides suspected of being present in a given sample, and (2) subjecting the extant polypeptides to a set of promiscuous affinity reagents that, taken as a whole, are expected to bind each candidate polypeptide in a different combination, such that each candidate polypeptide is expected to generate a unique profile of binding and non-binding events. Promiscuity of an affinity reagent can arise due to the affinity reagent recognizing an epitope that is known to be present in a plurality of different candidate polypeptides. For example, epitopes having relatively short amino acid lengths such as dimers, trimers, tetramers or pentamers can be expected to occur in a substantial number of different polypeptides in a typical proteome. Alternatively or additionally, a given promiscuous affinity reagent may recognize multiple different epitopes (e.g., epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

Although performing a single binding reaction between a promiscuous affinity reagent and a complex polypeptide sample may yield ambiguous results regarding the identity of the different extant polypeptides to which it binds, the ambiguity can be resolved by decoding the binding profiles for each extant polypeptide using machine learning or artificial intelligence algorithms that are based on probabilities for the affinity reagents binding to candidate polypeptides. For example, a plurality of different promiscuous affinity reagents can be contacted with a complex population of extant polypeptides, wherein the plurality is configured to produce a different binding profile for each candidate polypeptide suspected of being present in the population. The plurality of promiscuous affinity reagents can produce a binding profile for each extant polypeptide that can be decoded to identify a unique combination of positive outcomes (i.e. observed binding events) and/or negative binding outcomes (i.e. observed non-binding events), and this can in turn be used to identify the extant polypeptide as a particular candidate polypeptide having a high likelihood of exhibiting a similar binding profile.

Binding profiles can be obtained for extant polypeptides and decoded. In many cases one or more binding events produces inconclusive or even aberrant results and this, in turn, can yield ambiguous binding profiles. For example, observation of binding outcome at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate polypeptides that are suspected of being present in an assay will have produced an empirically observed binding profile. The binding model can include information regarding expected binding outcomes (e.g., positive binding outcomes and/or negative binding outcomes) for one or more affinity reagents with respect to one or more candidate polypeptides. A binding model can include a measure of the probability or likelihood of a given candidate polypeptide generating a false positive or false negative binding result in the presence of a particular affinity reagent, and such information can optionally be included for a plurality of affinity reagents. Decoding can be configured to evaluate the degree of compatibility of one or more empirical binding profiles with results computed for various candidate polypeptides using a binding model. For example, to identify an extant polypeptide in a sample, an empirical binding profile for the extant polypeptide can be compared to results computed by the binding model for many or all candidate polypeptides suspected to be in the sample. A machine learning or artificial intelligence algorithm can be used. An algorithm used for decoding can utilize Bayesian inference. In some configurations, identity for an extant polypeptide is determined based on a likelihood of the extant polypeptide being a particular candidate polypeptide given the empirical binding pattern or based on the probability of a particular candidate polypeptide generating the empirical binding pattern. Particularly useful decoding methods are set forth, for example, in U.S. Pat. No. 10,473,654; U.S. Pat. App. Pub. No. 2020/0318101 A1; U.S. patent application Ser. No. 18/045,036, or Egertson et al., BioRxiv (2021), DOI: 10.1101/2021.10.11.463967, each of which is incorporated herein by reference.

One or more compositions set forth herein can be present in an apparatus or vessel. For example, one or more affinity reagent(s) of the present disclosure can be present in a vessel, such as a flow cell. As a further option, the vessel can be engaged with a detection apparatus. The vessel can be permanently or temporarily engaged with the detection apparatus. A detection apparatus can be configured to detect contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g., a laser or lamp) then through a window of the vessel. The detection apparatus can further include a camera or other detector that acquires signals transmitted through the window of the vessel and through an optical train. Optionally excitation and emission can be transmitted through the same optical train; however, separate optical trains can also be useful.

A detection apparatus can include a fluidics system, for example, configured for fluidic communication with a vessel, such as a flow cell. In some configurations, a detection apparatus can include one or more reservoirs containing affinity reagents or analytes that are delivered to a vessel. The one or more reservoirs can contain affinity reagents that recognize one or more epitopes in an array of analytes, such as polypeptide analytes, in the vessel. Optionally, a detection apparatus can be configured to include a waste receptacle to which waste from the vessel is collected. For example, affinity reagents can be delivered from the apparatus through an ingress of a flow cell and waste can be removed through an egress of the flow cell to the apparatus. As such, a detection apparatus can be configured to deliver to a flow cell (or other vessel) affinity reagents that recognize one or more epitopes in an analyte or in an array of analytes.

One or more compositions set forth herein can be provided in kit form including, if desired, a suitable packaging material. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular polypeptide or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection apparatus. For example, the cartridge can be engaged with a detection apparatus such that contents of the cartridge are in fluidic communication with the detection apparatus or with a flow cell engaged with the detection apparatus. A cartridge can be engaged with a detection apparatus such that contents of the cartridge can be observed by the detection apparatus, for example, using an assay set forth herein.

A kit, cartridge or detection apparatus can optionally be configured to include one or more vessels containing an affinity reagent (labeled or unlabeled), artificial polymer, antibody, analyte (e.g., polypeptide analyte), flow cell, array of analytes and/or other composition set forth herein. In some cases, a kit or detection apparatus can include a plurality of different affinity reagents, each of the different affinity reagents having affinity moieties that recognize or bind to different epitopes, respectively. The different affinity reagents can optionally have the same structure for their respective artificial polymers. The different affinity reagents can further include labels that produce overlapping signals and in some cases the labels can have the same structure. Alternatively, different affinity reagents can be distinguishable due to having different labels that produce distinguishable signals.

Optionally, a kit, cartridge or detection apparatus can include (a) a first affinity reagent having a first artificial polymer attached to a first affinity moiety and a first label, wherein the artificial polymer has a branched chain structure; and (b) a second affinity reagent having a second artificial polymer attached to a second affinity moiety and a second label, wherein the second artificial polymer includes the branched chain structure, wherein the first affinity moiety recognizes an epitope that is not recognized by the second affinity moiety and the second affinity moiety recognizes an epitope that is not recognized by the first affinity moiety. The first label can differ from the second label in terms of signal produced or structure. Alternatively, the first and second labels can produce overlapping signals, for example, due to having the same chemical structures.

In one configuration of the above kit, cartridge or detection apparatus, the first affinity reagent is contained in a first vessel and the second affinity reagent is contained in a second vessel. Alternatively, the first affinity reagent and the second affinity reagent are mixed in a vessel. A kit or detection apparatus can include at least 1, 2, 5, 10, 25, 50, 100, 250, 500 or more different affinity reagents.

### EXAMPLES

### Abbreviations

Aldehyde, Ald
2-Amino-5-methoxybenzoic acid, AMBA
Azide, N₃
azobis(2-methylpropionamidine) hydrochloride, AAPH
Borate-buffered saline, BBS
1,1′-carbonyldiimidazole, CDI
dibenzocyclooctyne, DBCO
N,N-diisopropyl-N-ethylamine, DIPEA
dimethylformamide, DMF
dimethylsulfoxide, DMSO
N-Ethyl-N′-(3-dimethylaminopropyl)carbodiimide hydrochloride, EDC
Equivalent, Eq
Fluorescence-linked immunosorbent assay, FLISA
generation 3, G3
generation 5, G5
Glutaric acid, GA
HC, heavy chain of antibody
high performance liquid chromatography, HPLC
Hydrazinenicotinate, HyNic
4′-Hydroxyazobenzene-2-carboxylic acid, HABA
N-hydroxysuccinimide, NHS
Inverse Electron Demand Diels-Alder, IEDDA
Labeled probe (or labeled affinity reagent), Lobe
LC, light chain of antibody
methanol, MeOH
Methyltetrazine, mTz
molecular weight cut-off, MWCO
Non-specific binding, NSB
Origami buffer, OB
phosphate-buffered saline, PBS
polyacrylamide gel electrophoresis, PAGE
poly(amidoamine), PAMAM
poly(ethyleneglycol), PEG
polyethyleneimine, PEI
Protein A, PA
room temperature, rt
SDS, sodium dodecylsulfate
Size exclusion chromatography, SEC
Streptavidin, SA
strain-promoted azide-alkyne click, SPAAC
Tetrazine, Tz
2,3,5,6-tetrafluorophenol, TFP
Trans-cyclooctene, TCO
total internal reflection fluorescence, TIRF
tris(2-carboxyethyl)phosphine hydrochloride, TCEP

### Example I

### Synthesis of PEI(propargyl)ₙ₌₀(azide)ₙ₌₁₅(AZ488)_{o=0.05}

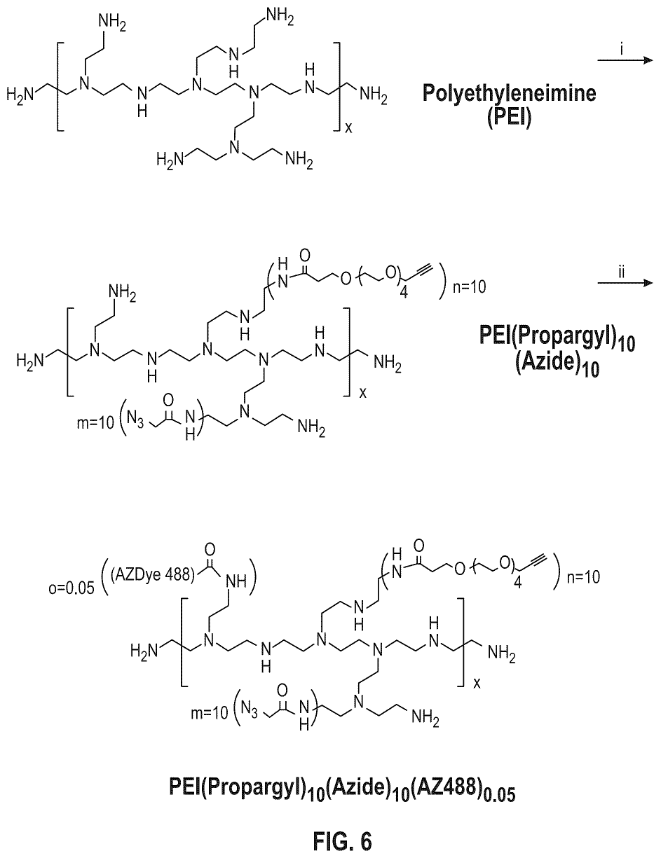

This example describes a representative route involved in the synthesis of a polyethyleneimine (PEI) polymer co-functionalized with alkyne and azide (FIG. 6). Its functionalization with two orthogonal moieties allows PEI attachment to an antibody as well as post labeling with a plurality of fluorescent dye molecules through DBCO/azide-paired strain-promoted azide-alkyne click (SPAAC) conjugation.

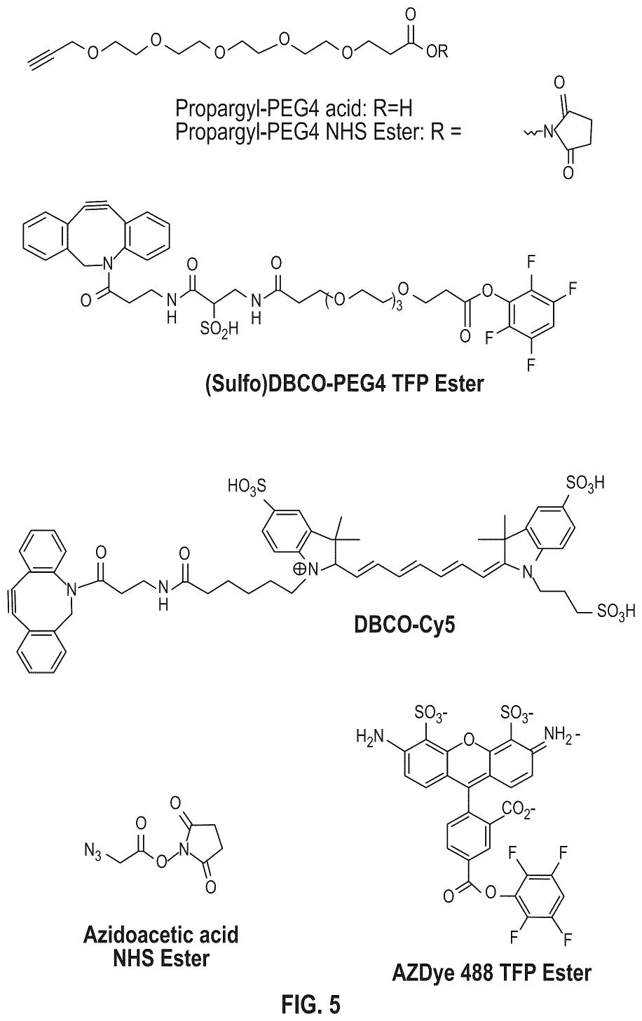

Activation of alkyne-PEG4-acid. Alkyne-PEG4-acid (8.9 mg, 27.8 mmol; 10 eq to PEI) (FIG. 5) was dissolved in anhydrous acetonitrile (0.2 mL). To this solution was added a solution of CDI(5.4 mg, 33.3 μmol; 1.2 eq to alkyne acid) dissolved in 0.05 mL of acetonitrile. This mixture was stirred at room temperature for 2.5 h. After activation, it was mixed with azidoacetic acid NHS ester (5.5 mg, 27.8 μmol; 10 eq to PEI) in an Eppendorf vial.

The solution of two activated esters which were mixed together was then added into a poly(ethyleneimine) (0.1 mL, 114 μmol; Mₙ 1800) solution prepared in 10% aqueous acetonitrile (5% w/v). This mixture was stirred for 15 min prior to adding a solution of AZDye488 TFP ester in DMSO (95 μL, 1 mg/mL; 0.05 eq to PEI). The final mixture was stirred overnight and concentrated in a Centrivap™ centrifugal concentrator. Its residue was dissolved in water and adjusted to 0.3 mL. This solution was divided into two desalting columns (Zeba™) and spin filtered at 4000 rpm for 2 min. Each filtrate was collected, loaded into a new desalting column and spin filtered at 400 rpm for 2 min. This filtration process was repeated three more times and filtrates were collected. Reverse-phase HPLC: retention time tᵣ=5.2-6.6 min (broad).

### Example II

### Synthesis of PEI(sulfo-DBCO)ₙ₌₁₀(DL650)ₘ₌₁₅

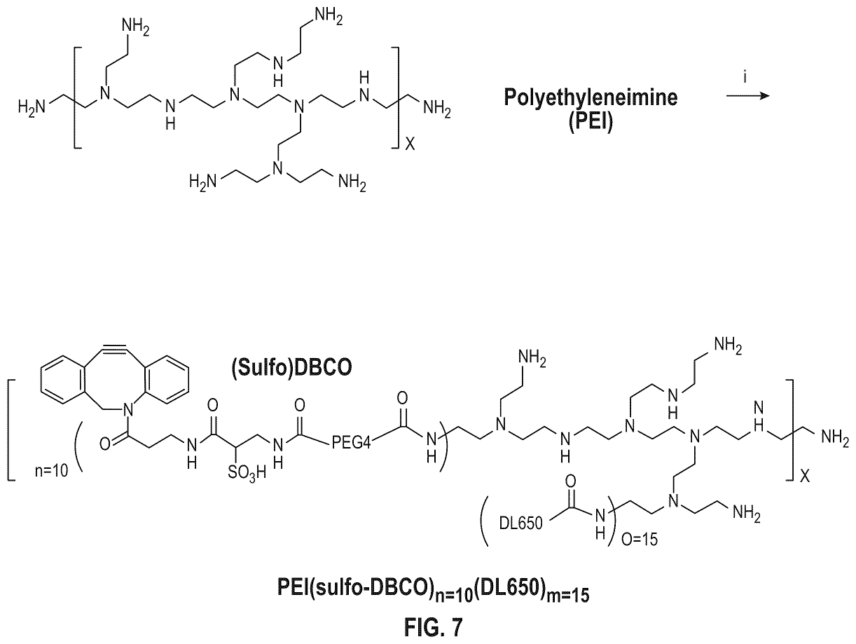

This example describes a representative route involved in the synthesis of a polyethyleneimine (PEI) polymer co-functionalized with DBCO and a fluorescent dye molecule (FIG. 7). Such functionalization allows PEI attachment to antibody (antibody-PEI) through photo thiol-yne conjugation or PEI attachment to antibody pre-conjugated with azide-carrying PEI (antibody-PEI-PEI) through DBCO/azide-paired strain-promoted azide-alkyne click (SPAAC) conjugation. Co-modification with a plurality of DL650 fluorescent labels allows fluorescent detection and imaging of PEI conjugates.

To a vial loaded with 0.05 mL of PEI (0.2 mg/mL; Mₙ=1800 g/mol) in MeOH was added a solution of (sulfo)DBCO-PEG4 TFP ester (4.7 μL, 11.7 mM; 10 eq). The mixture was vortexed shortly for 20 seconds which was followed by addition of DLdye 650 NHS ester in DMSO (89 μL, 1.0 mg/mL; 15 eq). This vial was wrapped with aluminum foil and gently shaken on a rocker at rt. After shaking overnight, the reaction was quenched by treatment with 0.2 mL of borate-buffered saline, pH 8.2. After shaking for 30 min, its volatile solvents were evaporated in a Centrivap centrifugal concentrator. Its residue was dissolved in 0.05 mL of water, wrapped with aluminum foil, and left at rt. This DBCO-modified PEI was used without any further treatment. Reverse-phase HPLC analysis: retention time=12.7 min (a broad peak; absorbance at 650 nm channel).

### Example III

### Synthesis of G3(propargyl)ₙ₌₇(azide)ₙ₌₁₆(AZ488)_{o=0.5}

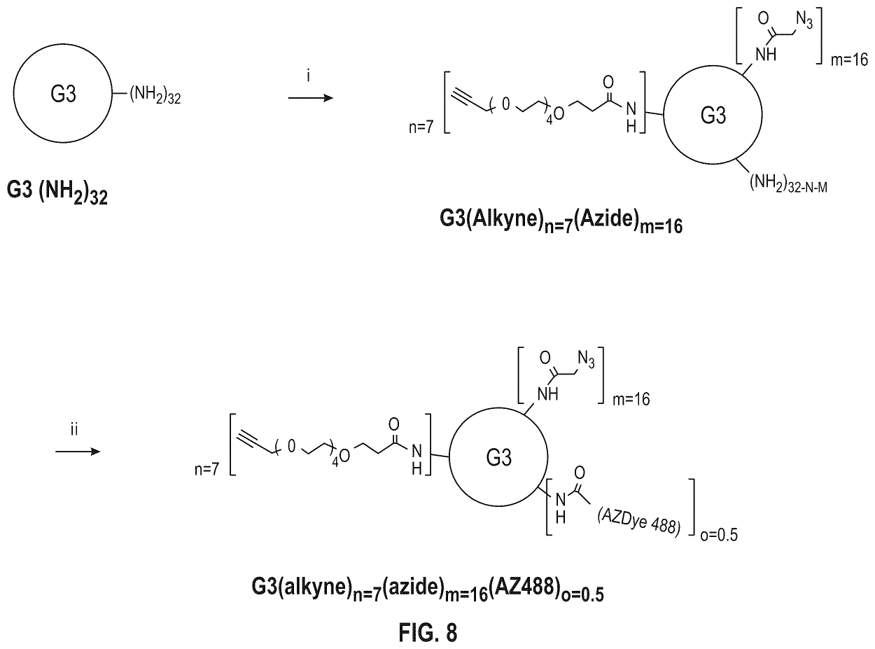

This example describes a representative route involved in the synthesis of poly(amidoamine) (PAMAM) dendrimer polymer co-functionalized with alkyne and azide (FIG. 8). Its functionalization with two orthogonal moieties allows PAMAM dendrimer attachment to an antibody as well as post labeling with a plurality of fluorescent dye molecules through DBCO/azide-paired strain-promoted azide-alkyne click (SPAAC) conjugation.

To a solution of generation 3 (G3) PAMAM dendrimer (Dendritech, Inc, Midland, MI)) in methanol (1.1 mL, 1.59 mmol; 10 mg/mL) was added N,N-diisopropyl-N-ethylamine (DIPEA) (8.9 mL, 32 eq) and then a mixture of propargyl PEG₄ NHS ester (4.5 mg, 11.1 mmol; 7 eq) and azidoacetic acid NHS ester (5.1 mg, 2.55 mmol; 16 eq). This mixture was shaken at 25° C. for 15 h and concentrated to an oily residue in a CentriVap centrifugal concentrator. This residue was re-dissolved in methanol (0.7 mL) for its final treatment with AZDye 488 TFP ester in DMF (0.04 mL, 0.80 mmol; 20 mM). This final mixture was shaken at 25° C. for 4 h. For product purification, the reaction mixture was concentrated in a CentriVap centrifugal concentrator, and its oily residue was re-dissolved in 1.0 mL of phosphate-buffered saline (PBS) pH 7.4. This solution was loaded in a spin filtration tube (Vivaspin® 2, Sartorius; membrane—molecular weight cutoff (MWCO) 5000) and spin filtered at 4000 g for 15 min. Its filtrate was discarded, and its concentrate was diluted with PBS (2.0 mL) prior to spin filtration at 4000 g for 15 min. This spin filtration process was repeated three additional times using PBS (3×2.0 mL). After filtration, its concentrate was recovered and adjusted to a volume of 0.5 mL using PBS. Multifunctional moieties attached at G3 dendrimer were characterized by UV-vis absorbance in a NanoDrop™ spectrophotometer (Thermo Scientific): 488, 340, 300, 280 nm; Ab (280 nm)=3.7 at 1.64 mg/mL in PBS, pH 7.4.

### Example IV

### Fluorescent Labeling of G3(Propargyl)(Azide)(AF488) by Strain-Promoted Azide-Alkyne Click (SPAAC) Conjugation

The plurality of azide moieties tethered at G3(propargyl)ₙ₌₇(azide)ₙ₌₁₆(AZ488)_{o=0.5} can serve as a chemical handle which is applicable for fluorescent dye labeling. This can be achieved by a click reaction with DBCO-Cy5 through strain-promoted azide-alkyne click (SPAAC) conjugation. To demonstrate this, G3(propargyl)₇(azide)₁₆(AZ488)_{0.5} was treated with DBCO-Cy5 by varying their molar ratios, and their reaction mixtures were characterized by sodium dodecylsulfate (SDS)-polyacrylamide gel electrophoresis (PAGE).

SDS-PAGE analysis was performed in a mini cassette electrophoresis kit using pre-casted Bolt™ Bis-Tris Plus polyacrylamide gels (4-12%; Invitrogen by Thermo Scientific) and a 2-(N-morpholino)ethanesulfonate (MES) running buffer. Each sample was prepared in a sample loading buffer (Bolt™, 1×lithium dodecylsulfate (LDS) sample buffer; Novex) and loaded onto the SDS-PAGE gel. Gels were loaded in a mini gel tank apparatus (Bio-Rad) and electrophoresis was performed at constant 200 V for 22 min (room temperature). After electrophoresis, each gel was removed from its plastic cassette, rinsed with water, and photographed or imaged for fluorescent bands in a ChemiDoc™ MP imaging system (Bio-Rad). After imaging, the gel was fixed for 10 min in 10% acetic acid, 30% ethanol in water, treated for silver staining using a Pierce™ silver stain kit (Thermo Scientific) according to its instruction and imaged in the imaging system (Bio-Rad).

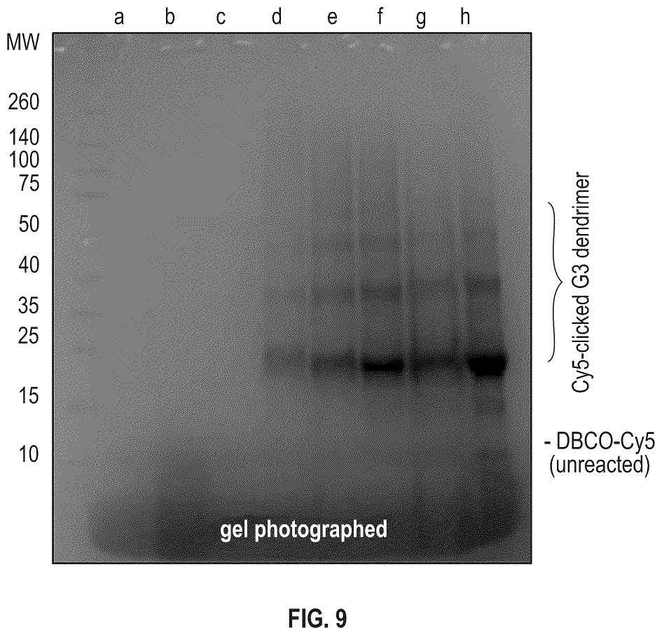

The gel image in FIG. 9 shows that controls such as a reaction mixture from the unmodified G3 dendrimer (lane b) or a reaction mixture not treated with DBCO-Cy5 (lane c) lack dye-labeled bands. Such negative controls are contrasted with those reaction mixtures from G3(propargyl)₇(azide)₁₆(AZ488)_{0.5} in each of which dye labeled bands are visible and growing in their intensity in response to the ratio of DBCO-Cy5 added. This SPAAC result is supportive of effective Cy5 dye conjugation at the azide moieties of G3(propargyl)₇(azide)₁₆(AZ488)_{0.5.}

### Example V

### Synthesis of Antibody-Polyethyleneimine Constructs

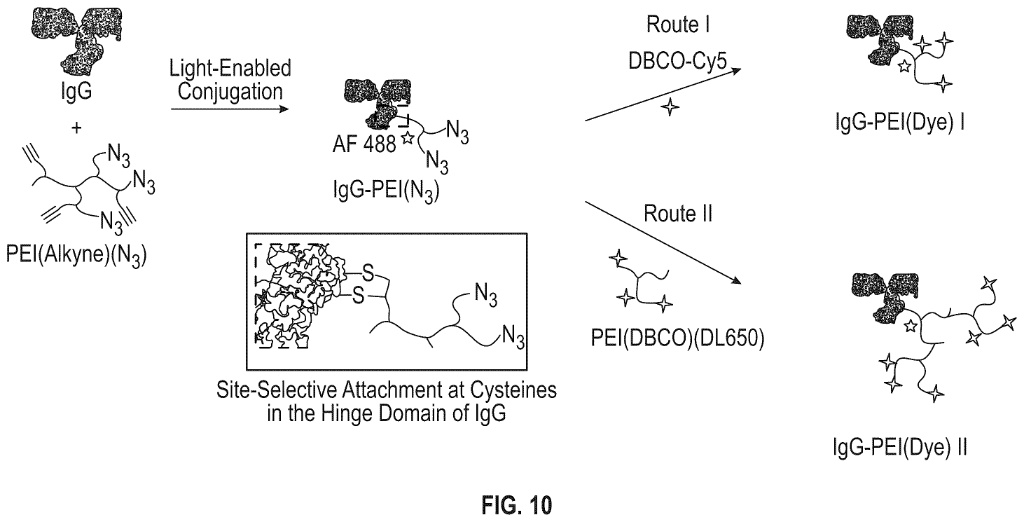

This example describes two routes for synthesis of an affinity reagent having a polyethyleneimine polymer attached to an antibody and a plurality of luminescent labels. FIG. 10 shows a diagram of the two synthesis routes. The first step is common to both routes. In the first step, the sulfurs in a pair of cysteines of an IgG antibody are reacted with an alkyne moiety of a polyethyleneimine that is co-functionalized with alkyne and azide moieties (PEI(alkyne)(N₃)). The reaction is performed under irradiation with light and produces an IgG-PEI(N₃) conjugate in which two carbons of the PEI alkyne moiety form a bridge between the sulfurs of the pair of cysteines (insert). The PEI(alkyne)(N₃) can optionally include an attached Alexa Fluor 488 dye (AF 488) for tracking purposes only that is carried through on the IgG-PEI(N₃) conjugate. The two different routes diverge in the second step. In the first route of the second step, shown in the upper reaction of FIG. 5, a Cy5 dye having a dibenzocyclooctyne moiety (DBCO-Cy5; FIG. 5) is reacted with an azide moiety of the IgG-PEI(N₃) conjugate via a click reaction to form an IgG-PEI conjugate having attached Cy5 dyes (IgG-PEI(dye) I). In the second route of the second step, shown in the lower reaction of FIG. 5, a PEI molecule having a plurality of dye moieties and a DBCO moiety (PEI(DBCO)₁₀(DL650)₁₅; FIG. 7) is reacted with the IgG-PEI conjugate to form a second antibody-PEI conjugate with DL650 dyes attached (IgG-PEI(dye) II). The IgG-PEI(dye) II product is larger than the IgG-PEI(dye) I due to the larger PEI moiety.

### Example VI

### Synthesis of Anti-HPD Antibody-PEI Constructs—Route I

This example describes one of the synthetic routes described in EXAMPLE V (FIG. 10). This route was applied to synthesizing an affinity reagent, anti-HPD IgG-PEI(dye) I, in which its PEI polymer is attached to an antibody with a plurality of luminescent labels.

Antibody-PEI polymer conjugation. An anti-HPD IgG solution in PBS pH 7.2 (0.2 mL, 1.0 mg/mL; 5.56 μM) was treated with a solution of tris(2-carboxyethyl)phosphine (TCEP) in water (4.5 μL, 2.5 mM). This condition allowed site-selective reduction of its disulfide bonds to cysteine thiols in its Fc domain by which these vicinal thiols can engage in photo thiol-yne conjugation with an alkyne moiety in PEI(propargyl)(azide). After TCEP addition, its mixture was incubated overnight in a refrigerator. To this TCEP-treated anti-HPD IgG was added PEI(propargyl)₁₀(azide)₁₅(AZ488)_{0.5} (23.8 μL, 0.93 mM; 20 eq to IgG) and AAPH (12 μL, 18.5 mM; 200 eq). This mixture was vortexed, spun down and left for 5 min in an ice bath prior to proceeding to irradiation. The irradiation was performed in an ice bath for 15 min at 365 nm (long wavelength UVA) using a light source of Asahi Spectra Max-350. After irradiation, this solution was treated with a H₂O₂ solution (12 μL; 98 mM) and left in an ice bath for 2 h. The H₂O₂-treated antibody solution was purified by spin filtration through a membrane tube (Amicon ultra −0.5 mL, MWCO 100 k) at 8000 rpm, 5 min (Eppendorf model 5430/5430R). Its filtrate was discarded while its concentrate which contained the IgG-PEI polymer was diluted by replenishing 0.4 mL of PBS prior to spin filtration. This spin filtration was repeated three additional times and the concentrated solution remaining in the upper membrane tube was recovered to afford a conjugation product IgG-PEI (95 μL).

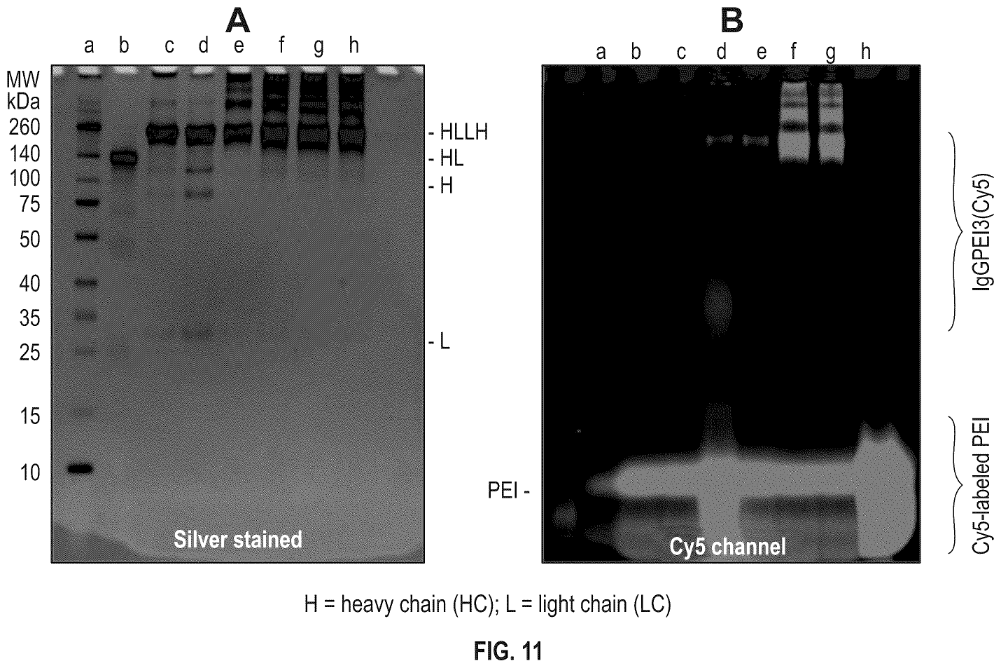

Cy5 dye conjugation through SPAAC reaction. To a vial that contained the IgG-PEI polymer prepared above (85 μL, 8.7 μM) was added a DBCO-Cy5 solution (75 μL, 0.5 mM). This reaction mixture was incubated while shaking at 25° C. for 15 h. After incubation, this mixture was purified by membrane filtration through an Amicon tube (MWCO 100 k) which was pre-washed with PBS (0.3 mL). This tube was spun down at 8000 rpm for 5 min and its filtrate was discarded. After this filtration, its concentrate, which contained the Cy5 dye-labeled IgG-PEI conjugate, was recovered and adjusted to a volume of 0.4 mL using PBS. This Cy5-labeled IgG-PEI conjugate was further purified by treatment with protein A magnetic beads (Pierce™) according to the manufacturer's protocol and recovered (0.12 mL, 5.0 μM). UV-vis absorbance: 650 (Xmax), 600, 280 nm. The presence of Cy5 dye molecules conjugated at IgG-PEI polymer was verified by SDS-PAGE gel analysis (FIG. 11) which was performed as described above in EXAMPLE 4. Its dye conjugation is supported by the appearance of new fluorescent bands in lane e (purified by spin filtration) or lane f (purified by protein A magnetic beads), each attributable to antibody heavy (H) and light (L) chains conjugated with Cy5-labeled PEI.

### Example VII

### Synthesis of Antibody-Polyethyleneimine Constructs—Route II

This example describes a second route of synthesizing an affinity reagent as described in FIG. 9. In this route, a multifunctional PEI polymer carrying a plurality of DBCO and luminescent labels is conjugated to a pre-formed antibody-PEI(azide) construct through DBCO-azide SPAAC.

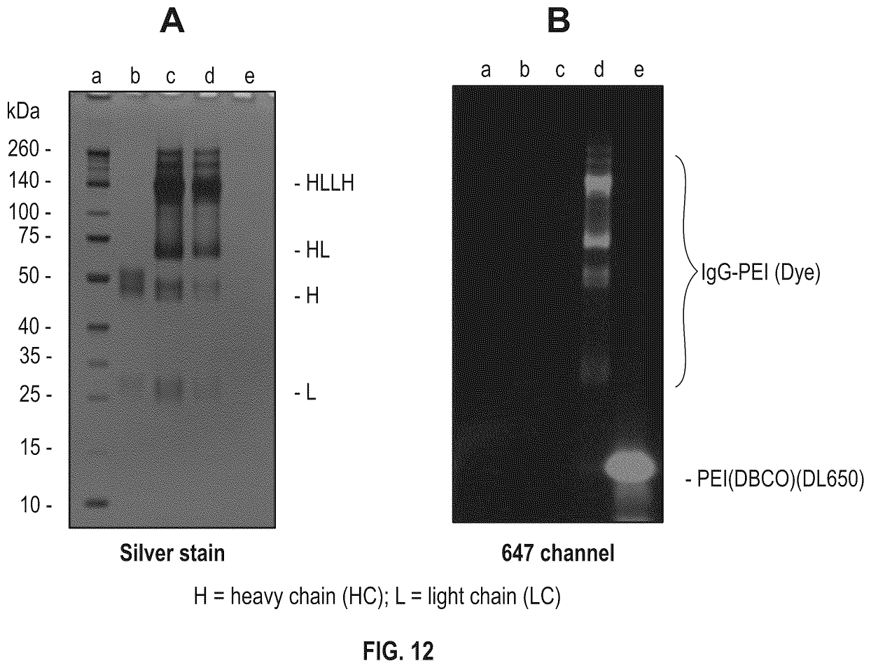

Conjugation of IgG-PEI(azide) with PEI(sulfo-DBCO)(DL650). To a solution of pre-immune IgG-PEI(azide) conjugate (20 μL, 5.5 μM; FIG. 10) in an Eppendorf vial was added a solution of PEI(sulfo-DBCO)₁₀(DL650)₁₅ (1.11 nmol, 10 eq to IgG-PEI; see FIG. 7). The mixture was vortexed, spun down and followed by shaking at 35° C. for 2 h and at room temperature overnight in the dark (covered with aluminum foil). After incubation, this mixture was purified by spin filtration through a desalting column (Zeba™ MWCO 7 k, 0.5 mL capacity) at 1500 g for 2 min (Eppendorf 5430/5430R). After desalting filtration, its filtrate which contained PEI(DL650)-conjugated IgG-PEI was recovered. It was diluted with 0.2 mL of PBS and further purified by membrane filtration through an Amicon tube (MWCO 100 k) pre-washed with PBS (0.4 mL). After spinning for 5 min at 8000 rpm, the concentrate in the upper tube was collected by centrifugation for 3 min at 8000 rpm, which afforded 0.036 mL of the PEI(DL650)-conjugated IgG-PEI. Production of this conjugation product was verified by SDS-PAGE gel analysis (FIG. 12) as evident with the induction of fluorescent bands attributable to antibody-PEI species including heavy (H) and light (L) chains conjugated with PEI(DL650).

### Example VIII

### Synthesis of Anti-DTR Antibody-G3 Dendrimer Constructs

This example describes a synthetic route to an affinity reagent having a G3 dendrimer polymer attached to an antibody and a plurality of luminescent labels. As its antibody, anti-DTR IgG was conjugated with G3(propargyl)ₙ₌₇(azide)ₘ₌₁₆(AZ488)_{o=0.5} under a photo thiol-yne condition (FIG. 9A).

IgG-G3 polymer conjugation. An anti-DTR IgG solution in PBS pH 7.2 (0.2 mL, 1.0 mg/mL) was treated with a solution of tris(2-carboxyethyl)phosphine (TCEP) in water (13.3 μL, 2.5 mM) and incubated at 5° C. for 5 h. This condition allowed site-selective reduction of its disulfide bonds to cysteine thiols in its Fc domain. After TCEP reduction, this antibody solution was treated with G3(propargyl)₇(azide)₁₆(AZ488)_{0.5} (4.6 μL, 2.37 mM; 10 eq to IgG) and AAPH (20 μL, 18.5 mM; 200 eq). This mixture was vortexed, left for 5 min in an ice bath and irradiated in an ice bath for 10 min at 365 nm (long wavelength UVA light; Asahi Spectra Max-350). After this irradiation, a second portion of AAPH (20 μL, 18.5 mM; 200 eq) was added to the irradiated mixture and a second irradiation at 365 nm for 10 min continued. After irradiation, this solution was treated with a H₂O₂ solution (12 μL; 98 mM), left in an ice bath for 2 h and concentrated by spin filtration through a membrane tube (Amicon ultra—0.5 mL, MWCO 100 k) at 8000 rpm, 5 min (Eppendorf model 5430/5430R). Its filtrate was discarded while its concentrate was replenished with 0.4 mL of PBS and spin filtered. This spin filtration process was repeated three additional times prior to recovering its concentrated solution remaining in the upper membrane tube. The recovered solution which contained the conjugation product, IgG-G3 polymer, was adjusted to a volume of 0.1 mL using PBS.

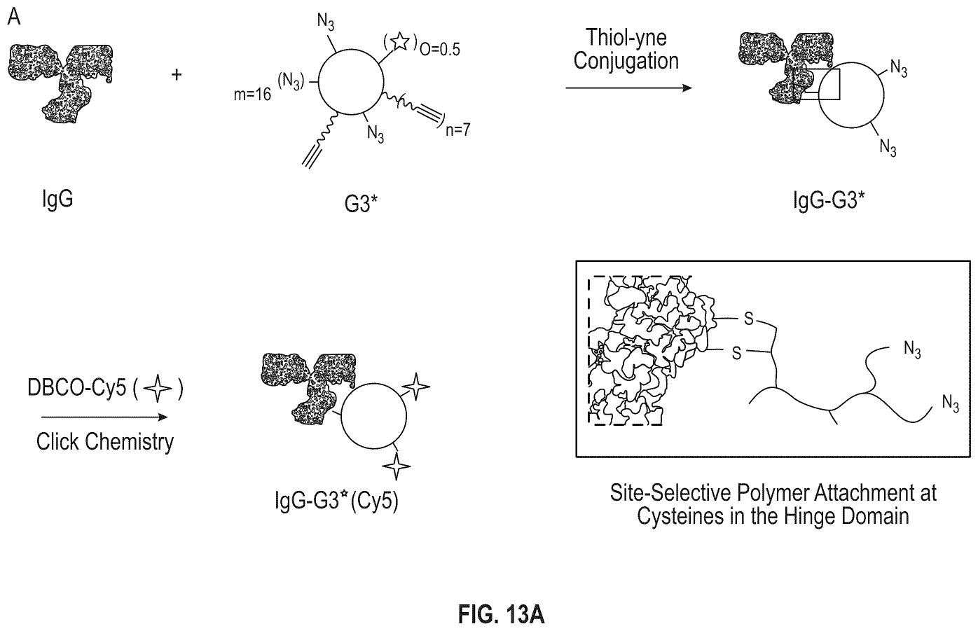

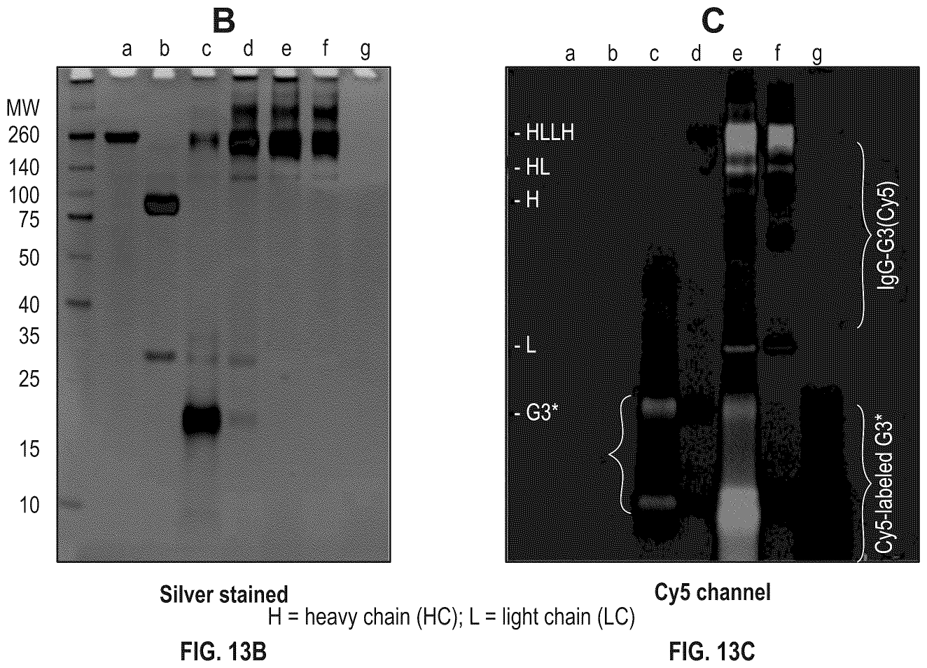

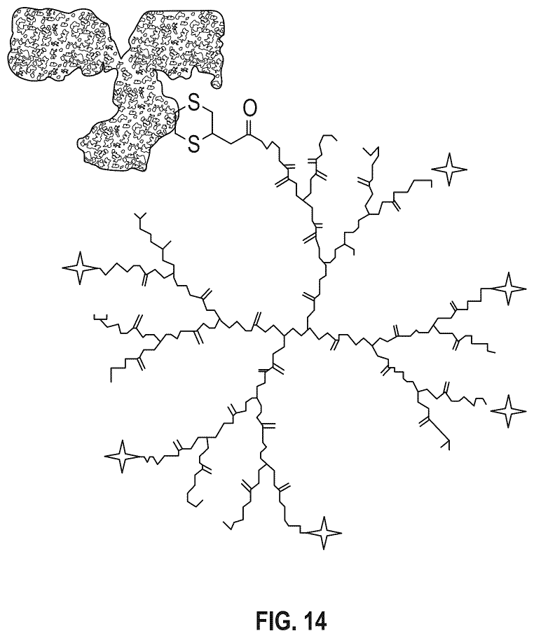

Cy5 dye conjugation through SPAAC reaction. To a vial that contained the IgG-G3 polymer prepared above (90 μL, 8.8 μM) was added a DBCO-Cy5 solution (16 μL; 1.0 mg/mL; 10% DMSO, PBS). This reaction mixture was incubated with shaking at 35° C. for 15 h. After incubation, this mixture was diluted with PBS (0.3 mL) and loaded onto a membrane tube (Amicon, MWCO 100 k) which was pre-washed with 0.05% tween-20, TBS saline. This tube was spun at 8000 rpm for 5 min and its filtrate was discarded. Its concentrate was diluted with 0.4 mL of 0.05% tween-20, tris-buffered saline prior to spin filtration at 8000 rpm for 5 min. After this filtration, its concentrate which contained Cy5 dye-labeled IgG-G3 conjugate was recovered and adjusted to a volume of 0.1 mL (4.7 μM) with PBS. This Cy5-labeled IgG-G3 conjugate was further purified by treatment with protein A magnetic beads (Pierce™) which was performed according to its manufacturer's protocol. UV-vis absorbance: 660 (λₘₐₓ), 610, 490, 335, 280 nm. The presence of Cy5 dye molecules conjugated at IgG-G3 polymer was verified by SDS-PAGE gel analysis which was performed as described above (EXAMPLE IV). Its dye conjugation is supported by fluorescent bands attributable to Cy5 (FIG. 13C) in which the antibody-G3(Cy5) product purified from the mixture of IgG-G3*+DBCO-Cy5 (lane e and lane f) shows Cy5-specific fluorescence. Cy5-labeled free G3* dendrimer was observed but it migrated relatively faster (lane g).

### Example IX

### Single-molecule Fluorescence Assay for Binding of Antibody-Polyethyleneimine Constructs to Polypeptides

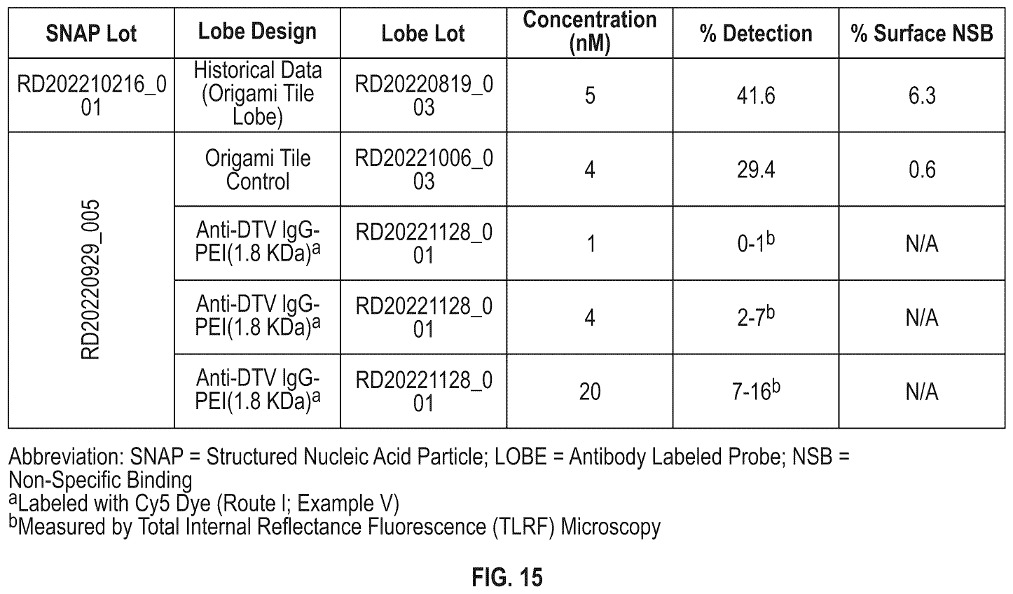

This example demonstrates use of antibody-IgG polymer conjugates as affinity reagents to detect polypeptides in an array of polypeptides. Their binding affinities to target epitopes were determined by a single-molecule fluorescence binding assay based on total internal reflection fluorescence (TIRF) microscopy as described in literature such as Fish, et al. Curr. Protoc. Cytom. 50:12.18.1-12.18.13 (2009), which is incorporated herein by reference. In a representative procedure, a series of test solutions of an antibody-polymer conjugate were prepared in a running buffer complemented with 1% bovine serum albumin and injected in a flow cell to allow its binding to a target peptide molecule presented on the surface of a structured nucleic acid particle (SNAP) deposited on the surface of the flow cell chip. After incubation for 1 hour, time-lapse images of fluorescent nanospots were collected using a 647-nm laser in a TIRF microscopy. Each of these nanospots is indicative of a peptide-bound antibody-polymer conjugate (co-localization). These were analyzed to determine the fraction of bound antibody-polymer populations relative to a total population of peptides presented on the chip surface. This fraction is referred to as the “detection rate” which is indicative of the extent of binding affinity to a single peptide target. A representative set of detection rates measured for anti-DTV IgG-PEI (MW 1.8 kDa) is summarized in FIG. 15. It suggests its ability to recognize its targeted peptide presented on the surface at a single-molecule level.

### Example X

### Synthesis of Aldehyde-Modified G3 Dendrimer G3(biotin)ₓ₌₁₅

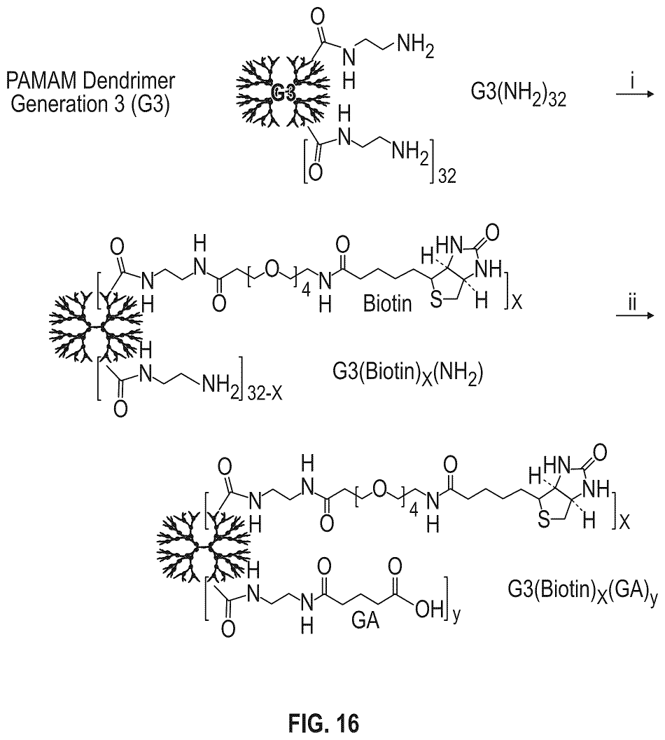

This example describes the synthesis of generation 3 (G3) poly(amidoamine) (PAMAM) dendrimer functionalized with biotin (FIG. 16). This biotin functionalization provides the PAMAM dendrimer with an ability to complex with streptavidin (SA).

To a solution of generation 3 (G3) PAMAM dendrimer (MW 6908 g/mol; Dendritech, Inc, Midland, MI) in methanol (0.11 mL; 10 mg/mL) was added N,N-diisopropyl-N-ethylamine (DIPEA) (1.1 μL, 40 eq) and then biotin PEG₄ NHS ester dissolved in acetonitrile (23.4 μL, 102 mM; 15 eq). This mixture was shaken at 25° C. for 6 h prior to treating with glutaric anhydride for derivatization of remaining amine branches to those terminated with glutaric acids. This was performed by adding DIPEA (10.9 μL, 500 eq) and glutaric anhydride (3.6 mg, 200 eq) and shaking at room temperature overnight.

After reaction, the mixture was concentrated to dryness in CentriVap™, and its residue was dissolved in 1×OB (1.0 mL). It was vortexed until its content was dispersed homogenously and sonicated for 1 min. This solution was loaded into a spin filtration tube (Sartorius, Vivaspin®, MWCO 5000), diluted with 1×OB (1.0 mL) and spin filtered at 5000 g (Eppendorf 5430/5430R) for 15 min. Its filtrate was discarded, and its concentrate was diluted with PBS pH 7.4 (2.0 mL) prior to spin filtration at 5000 g (15 min). This filtration process was repeated three additional times. After filtration, its concentrate was recovered and adjusted to a volume of 1.1 mL using PBS pH 7.4, affording 1.0 mg/mL (0.145 mM).

This biotin-modified G3 dendrimer G3(biotin)ₓ₌₁₅ was characterized for its identity and homogeneity by UV-vis (nanodrop) and HPLC analysis: max=230 nm; retention time (tᵣ)=7.79 min (HPLC). High performance chromatography (HPLC) was performed in an Agilent Technologies system (1260 Infinity II) equipped with a C4 column (Biozen™ 50×2.1 mm, Phenomenex). HPLC runs were performed using an eluent of aqueous acetonitrile (0.1% TFA, v/v) under a gradient condition (90% to 25% acetonitrile (0.1% TFA) in water (0.1% TFA) over 12 min) at a flow rate of 1.0 mL/min.

### Example XI

### Synthesis of Biotin-Modified G5 Dendrimer G5(biotin)ₓ₌₆₀

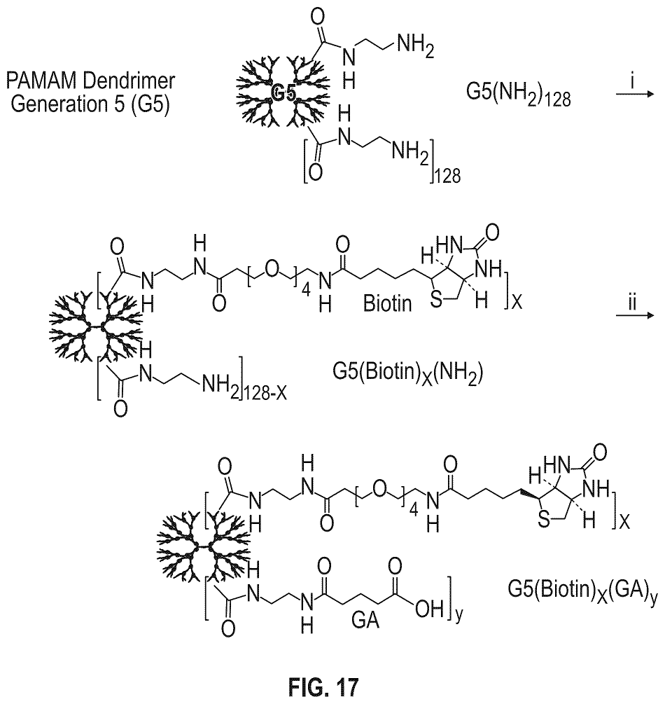

This example describes the synthesis of generation 5 (G5) PAMAM dendrimer functionalized with biotin (FIG. 17). This biotin functionalization provides the dendrimer with an ability to complex with streptavidin (SA).

To a solution of G5 dendrimer (MW 28,826 g/mol; Dendritech, Inc, Midland, MI) in methanol (0.1 mL; 20 mg/mL) was added N,N-diisopropyl-N-ethylamine (DIPEA) (2.5 μL, 200 eq) and then biotin PEG₄ NHS ester dissolved in 13% DMSO/acetonitrile (141 μL, 29.4 mM; 60 eq). This mixture was shaken at 25° C. for 18 h and treated with glutaric anhydride for derivatization of remaining amine branches to those terminated with glutaric acids. This was performed by adding DIPEA (4.7 μL, 500 eq) and glutaric anhydride (4.7 mg, 600 eq) and shaking it at room temperature overnight.

After reaction, the mixture was concentrated to dryness in CentriVap™, and its residue was dissolved in 1×OB (1.0 mL). It was vortexed until its content was dispersed homogenously and sonicated for 1 min. This solution was loaded into a spin filtration tube (Sartorius, Vivaspin® MWCO 5000), diluted with 1×OB (1.0 mL) and spin filtered at 5000 g for 15 min. Its filtrate was discarded, and its concentrate was diluted with PBS pH 7.4 (2.0 mL) and spin filtered at 5000 g (15 min). This filtration process was repeated three additional times. After filtration, its concentrate was recovered and adjusted to a volume of 1.0 mL using PBS pH 7.4, affording 2.0 mg/mL (0.0691 mM).

This biotin-modified G5 dendrimer G5(biotin)ₓ₌₆₀ was characterized for its identity and homogeneity by UV-vis (nanodrop) and HPLC analysis: λₘₐₓ=230 nm; retention time (tᵣ)=8.28 min (HPLC). HPLC was performed as set forth in Example X.

### Example XII

### Synthesis of Aldehyde-Modified G3 Dendrimer G3(Ald)ₓ₌₂₀

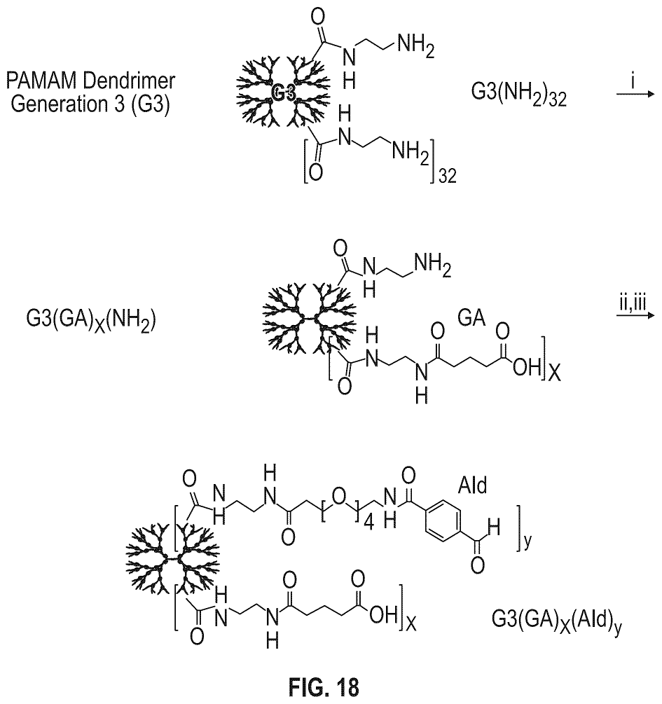

This example describes the synthesis of generation 3 (G3) PAMAM dendrimer functionalized with 4-formylbenzamide (FIG. 18). This aldehyde (Ald) functionalization provides the dendrimer with a reactivity selective with the functional moiety of hydrazine.

To a solution of G3 dendrimer (MW 6,908 g/mol; Dendritech, Inc, Midland, MI) in methanol (0.2 mL; 10 mg/mL) was added N,N-diisopropyl-N-ethylamine (DIPEA) (2.6 μL, 50 eq) and glutaric anhydride dissolved in 50% chloroform/acetonitrile (53 μL, 10 mg/mL; 16 eq). This mixture was shaken at 25° C. for 45 min and treated with 4-formylbenzamide PEG₄ TFP ester dissolved in acetonitrile (316 μL, 10 mg/mL; 20 eq). This mixture was shaken at 25° C. for 3 h and then treated with further glutaric anhydride for full derivatization of remaining amine branches to those terminated with glutaric acids. This was performed by adding DIPEA (2.6 μL, 50 eq) and glutaric anhydride dissolved in 50% chloroform/acetonitrile (66 μL, 10 mg/mL; 20 eq) and by shaking at 25° C. for 1 h.

After reaction, the mixture was concentrated to dryness in CentriVap™, and its residue was dissolved in PBS pH 7.4 (2.0 mL). It was loaded equally into two spin filtration tubes (Sartorius, Vivaspin® MWCO 5000), each diluted with PBS to a volume of 2.0 mL, and spin filtered at 5000 g for 15 min. Each filtrate was discarded, and its concentrate was diluted with PBS pH 7.4 (2.0 mL) and spin filtered under the same condition. This filtration process was repeated three additional times, and its concentrate was recovered and adjusted to a volume of 0.9 mL using PBS pH 7.4, affording 2.2 mg/mL.

This aldehyde-modified G3 dendrimer G3(Ald)ₓ₌₂₀ was characterized for its identity and homogeneity by UV-vis (nanodrop) and SDS-PAGE analysis. Sodium dodecylsulfate (SDS)-polyacrylamide gel electrophoresis (PAGE) was performed in a mini cassette electrophoresis kit loaded with pre-casted Bolt™ bis-tris plus polyacrylamide gels (4-12%) or NovexT^{M} tris-glycine polyacrylamide gels (6%), both from Thermo Scientific. Each sample was prepared by dilution in a sample loading buffer (Bolt™, 1×lithium dodecylsulfate (LDS) sample buffer; Novex) and loaded onto the SDS-PAGE gel. Gels were loaded in a mini gel tank apparatus (Bio-Rad) and electrophoresis was performed at constant 200 V for 22 min. After electrophoresis, each gel was removed from its plastic cassette, rinsed with water, and photographed and/or imaged for fluorescent bands (ChemiDoc™ MP imaging system; Bio-Rad). After imaging, the gel was fixed optionally for 10 min in 10% acetic acid, 30% ethanol in water, treated for protein staining using a kit for silver stain (Pierce™, Thermo Scientific), Lumitein™ (Biotium) or Imperial™ blue (Thermo Scientific), each according to its instruction, and proceeded to gel imaging (Bio-Rad).

The UV-vis spectral trace showed a strong absorbance in a range of 250 to 300 nm with λₘₐₓ of 260 nm. This is in good agreement with the contribution of benzaldehyde moieties if they are covalently attached to the dendrimer. The gel analysis indicated a single band with its position in close alignment with a standard marker of 15 kDa, a value close to its calculated MW of 14.8 kDa.

### Example XIII

### Synthesis of Aldehyde-Modified G5 Dendrimer G5(Ald)ₓ₌₅₀

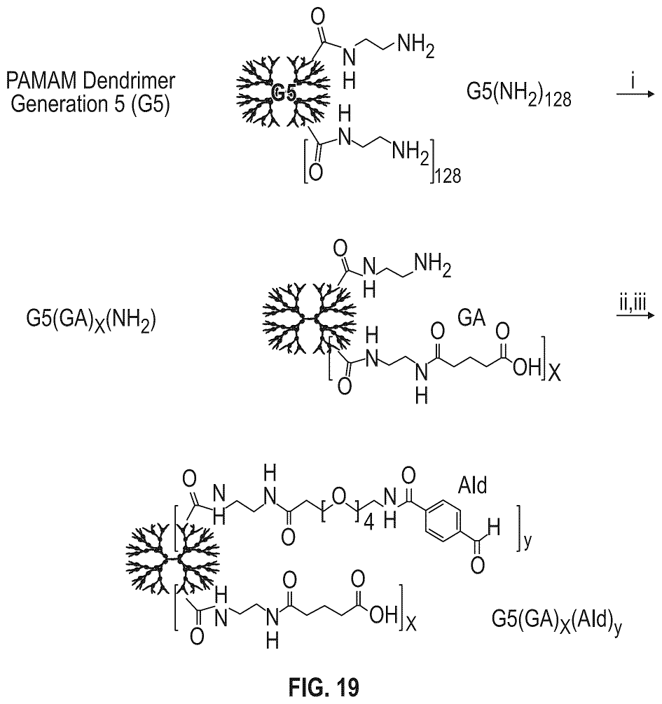

This example describes the synthesis of generation 5 (G5) PAMAM dendrimer functionalized with 4-formylbenzamide (FIG. 19). This aldehyde (Ald) functionalization provides the dendrimer with a reactivity selective with the functional moiety of hydrazine.

To a solution of G5 dendrimer (MW 28,826 g/mol; Dendritech, Inc, Midland, MI) in methanol (0.1 mL; 20 mg/mL) was added N,N-diisopropyl-N-ethylamine (DIPEA) (2.5 μL, 200 eq) and glutaric anhydride dissolved in 50% chloroform/acetonitrile (55 μL, 10 mg/mL; 70 eq). This mixture was shaken at 25° C. for 0.5 h and treated with 4-formylbenzamide PEG₄ TFP ester dissolved in acetonitrile (189 μL, 10 mg/mL; 50 eq). This mixture was shaken at 25° C. for 15 h and then treated with further glutaric anhydride for full derivatization of remaining amine branches to those terminated with glutaric acids. This was performed by adding DIPEA (9.5 μL, 1000 eq) and glutaric anhydride (3.9 mg; 500 eq) and by shaking at 25° C. for 1 h.

After reaction, the mixture was concentrated to dryness in CentriVap™, and its residue was dissolved in PBS pH 7.4 (2.0 mL). It was loaded equally into two spin filtration tubes (Sartorius, Vivaspin® MWCO 5000), each diluted with PBS to a volume of 2.0 mL, and spin filtered at 5000 g for 15 min. Each filtrate was discarded, and its concentrate was diluted with PBS pH 7.4 (2.0 mL) and spin filtered under the same condition. This filtration process was repeated three additional times, and its concentrate was recovered and adjusted to a volume of 0.45 mL using PBS pH 7.4, affording 4.4 mg/mL.

This aldehyde-modified G5 dendrimer G5(Ald)ₓ₌₅₀ was characterized for its identity and homogeneity by UV-vis (nanodrop) and SDS-PAGE analysis. SDS-PAGE analysis was performed as set forth in Example XII. The UV-vis spectral trace showed strong absorbance with λₘₐₓ of 260 nm, which is in good agreement with the contribution of benzaldehyde moieties present if they are covalently attached to the dendrimer. The gel analysis indicated a single band positioned between two standard markers each at 40 kDa and 50 kDa. This value is lower than its calculated MW of 53.5 kDa but indicative of a substantial increase from its parent value of 28.8 kDa. Its homogeneity is further supported by SEC-HPLC analysis in which a single peak appears at retention time (tᵣ) of 14.4 min. Size exclusion chromatography (SEC) HPLC was performed in an HPLC system (Hewlett Packard 1100 series) equipped with a size exclusion column (Bio SEC-5, Agilent BioHPLC) by eluting 1×origami buffer (OB; 5.0 mM Tris, 1.0 mM EDTA and 5.0 mM NaCl in water, pH −8.2) under an isocratic condition at a flow rate of 0.3 mL/min.

### Example XIV

### Synthesis of DBCO-Modified G5 Dendrimer G5(DBCO)ₓ₌₃₀

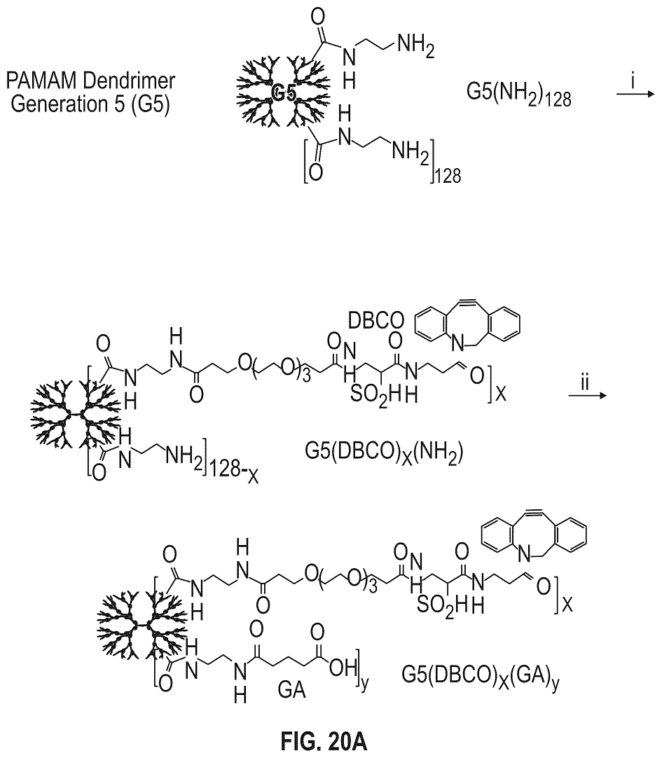

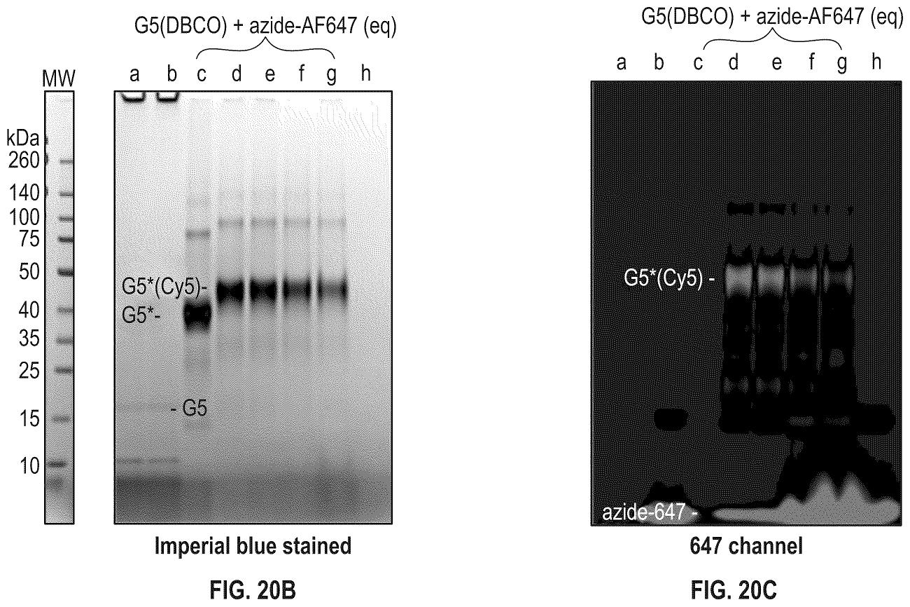

This example describes the synthesis of generation 5 (G5) PAMAM dendrimer functionalized with dibenzocyclooctyne (DBCO) (FIG. 20A). This DBCO functionalization provides the dendrimer with a reactivity selective with the functional moiety of azide through strain-promoted azide-alkyne click (SPAAC) conjugation.

A solution of G5 dendrimer (MW 28,826 g/mol; Dendritech, Inc, Midland, MI) in methanol (0.1 mL; 20 mg/mL) was mixed with N,N-diisopropyl-N-ethylamine (DIPEA) (2.5 μL, 200 eq). To this solution was added (sulfo)DBCO-PEG4 TFP ester dissolved in 50% DMSO/acetonitrile (177 μL, 10 mg/mL; 30 eq) in three portions. Immediately, this mixture was mixed well and shaken at 25° C. for 15 h. It was then treated with excess glutaric anhydride for full derivatization of remaining amine branches to those terminated with glutaric acids. This was performed by adding DIPEA (9.5 μL, 1000 eq) and glutaric anhydride (7.9 mg; 1000 eq) and by shaking at 25° C. for 1 h.

After reaction, the mixture was concentrated in CentriVap™ for 30 min. Its residue was mixed with 1×OB pH 8.2 (1.0 mL). Upon this dilution, its solution turned to cloudy suspension. It was vortexed for homogenous dispersion and sonicated for 1 min. It was loaded into a spin filtration tube (Sartorius, Vivaspin® MWCO 10000) pre-filled with 1×OB (1.0 mL) and spin filtered at 5000 g for 15 min. After discarding its filtrate, its concentrate was mixed with 1×OB (1.0 mL) and spin filtered under the same condition. After this spin filtration, the filtration process continued four additional times, each using PBS pH 7.4 instead of 1×OB. As its spin filtration continued, its solution turned to clear from its cloudy suspension. After filtration, its concentrate was recovered and its volume was adjusted to 1.0 mL using PBS pH 7.4, affording 2.0 mg/mL.

This DBCO-modified G5 dendrimer G5(DBCO)ₓ₌₃₀ was characterized for its identity and homogeneity by UV-vis (nanodrop) and SDS-PAGE analysis. Its UV-vis spectral trace shows strong absorbance with λₘₐₓ of 310 nm, 292 nm, which is indicative of the contribution of DBCO moieties present if covalently attached to the dendrimer. SDS-PAGE gel analysis was performed as set forth in Example XII. The results, shown in FIG. 20B and FIG. 20C, demonstrated that a single band positioned at 40 kDa. This value is slightly lower than its calculated MW of 49.4 kDa but represents a substantial increase from its parent value of 28.8 kDa. Its gel analysis is also supportive of the click reactivity of its DBCO with azide-AF647. Its homogeneity was characterized by HPLC analysis as set forth in Example X, and produced a single peak at the retention time (tᵣ) of 9.2 min.

### Example XV

### Synthesis of anti-DTR IgG Modified with Biotin IgG(Biotin)

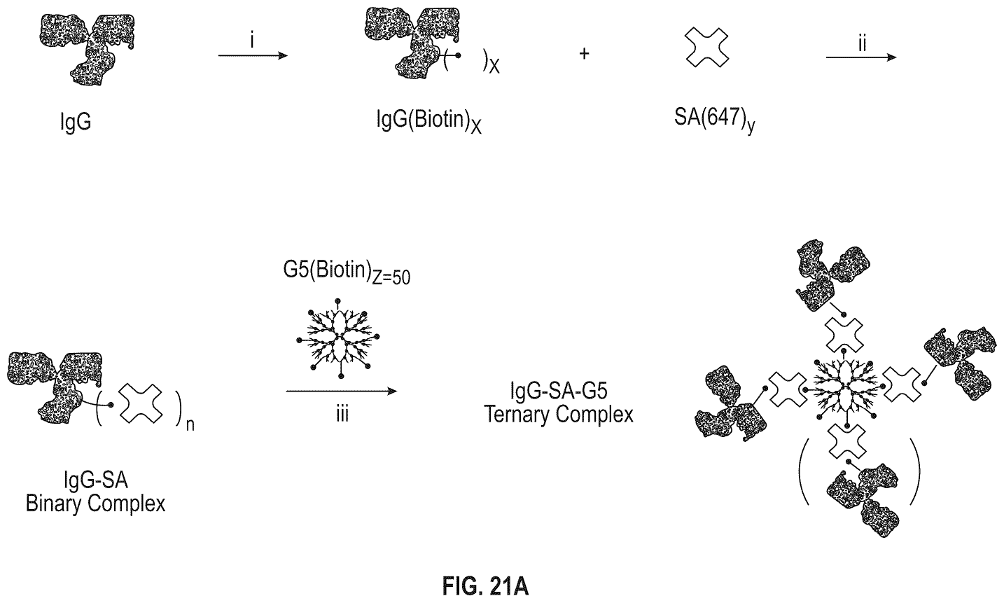

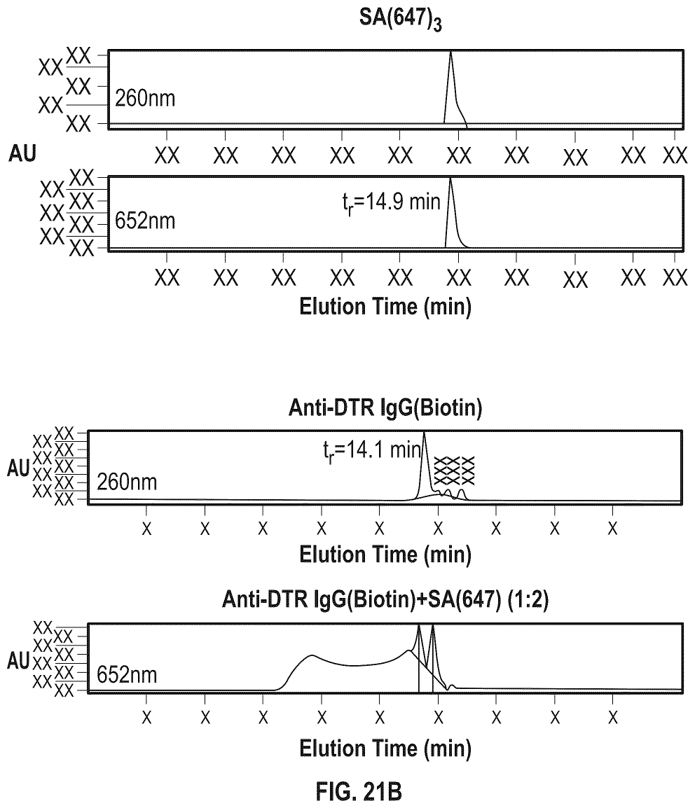

This example describes the synthesis of an IgG molecule functionalized with biotin (FIG. 21A). This biotin functionalization confers a streptavidin-specific binding affinity by which its antibody engages in complexation with streptavidin.

To a solution of anti-DTR IgG in borate-buffered saline (0.4 mL, 1.0 mg/mL) was added a solution of biotin-PEG₄ NHS ester dissolved in 10% DMSO/acetonitrile (3.3 μL, 17 mM; 25 eq). This mixture was shaken at 25° C. After shaking for 3 h, a second portion of the biotin-PEG₄ NHS ester (3.3 μL, 17 mM; 25 eq) was added to the reaction mixture, and this mixture was shaken for an additional period of 6 h. For purification, this mixture was loaded into a spin filtration tube (Sartorius, Vivaspin® MWCO 10,000; 2.0 mL capacity), diluted with 1×PBS (1.5 mL) and spun at 4200 g (Eppendorf 5430/5430R) for 12 min. Its filtrate was discarded, and its spin filtration was repeated four times. Its concentrate was recovered and adjusted to a volume of 0.25 mL. It was desalted by passing through two desalting columns (Zeba™, MWCO 7,000) at 4000 rpm (1900 g) and its collected volume was adjusted to 0.3 mL, affording IgG(biotin) (0.925 mg/mL; 69% yield). An average number of biotin residues attached to IgG was determined as 3.4±0.5 by HABA (4′-hydroxyazobenzene-2-carboxylic acid) assay performed using a fluorescence biotin quantitation kit (Pierce™, Thermo Fisher Scientific).

### Example XVI

### Preparation of Complexes of IgG(Biotin) with Streptavidin (AF647) and G5(Biotin)

This example describes methods that pertain to preparing a binary complex of IgG(biotin) with streptavidin(AZ647) and its ternary complexation with G5(biotin) (FIG. 21A).

An anti-DTR IgG(biotin) solution (100 μL, 5.83 mM) was mixed with a solution of streptavidin(AF647) (30.3 μL, 38.5 mM) which was added at 2 molar equivalent to IgG. This mixture was vortexed and shaken at 25° C. for 0.5 h, yielding a binary complex IgG(biotin)-SA. It was used without any treatment in the next step for preparing its ternary complex with biotin-modified G5 dendrimer G5(biotin)ₓ₌₅₀.

To the solution of a binary complex, IgG(biotin)-SA, prepared above (130 μL, [IgM]=4.47 μM) was added G5(biotin)ₓ₌₅₀ (26 μL, 5.59 μM) at a molar ratio of 4 IgG to 1 G5(biotin). This mixture was vortexed and shaken at 25° C. for 0.5 h, yielding a ternary complex, G5(biotin)@SA@IgG(biotin).

Formation of complexes either between IgG(biotin) and SA(647) or between SA(647) and G5(biotin) is supported by SEC-HPLC analysis (FIG. 21B), which was performed as set forth in Example X. A binary complex IgG(biotin)-SA shows a broad peak which is distinct in its peak shape and retention time (tᵣ) from its individual components, IgG(biotin) and SA(AF647), each showing a relatively sharp peak at tᵣ of 14.1 min and 14.9 min, respectively.

### Example XVII

### Preparation of G5(streptavidin) Conjugate Through Hydrazone Linkage

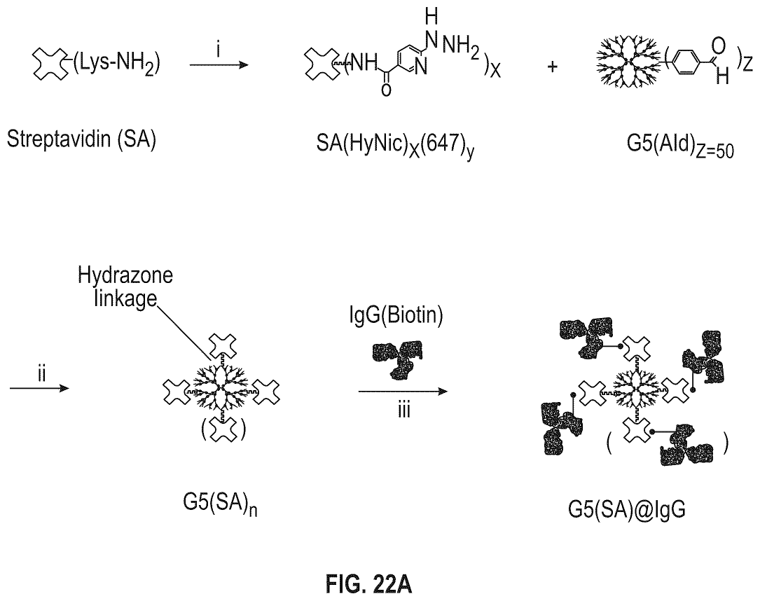

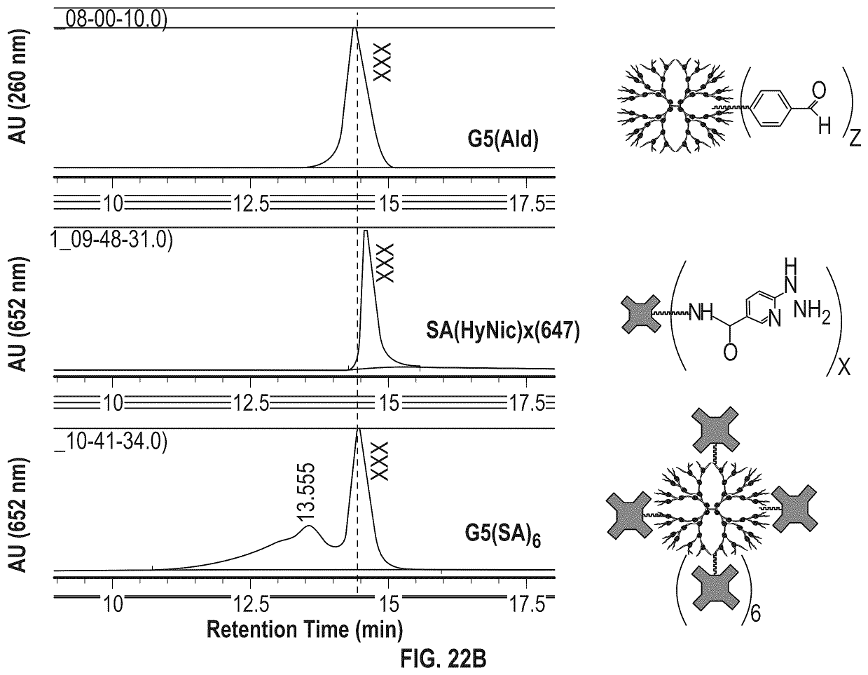

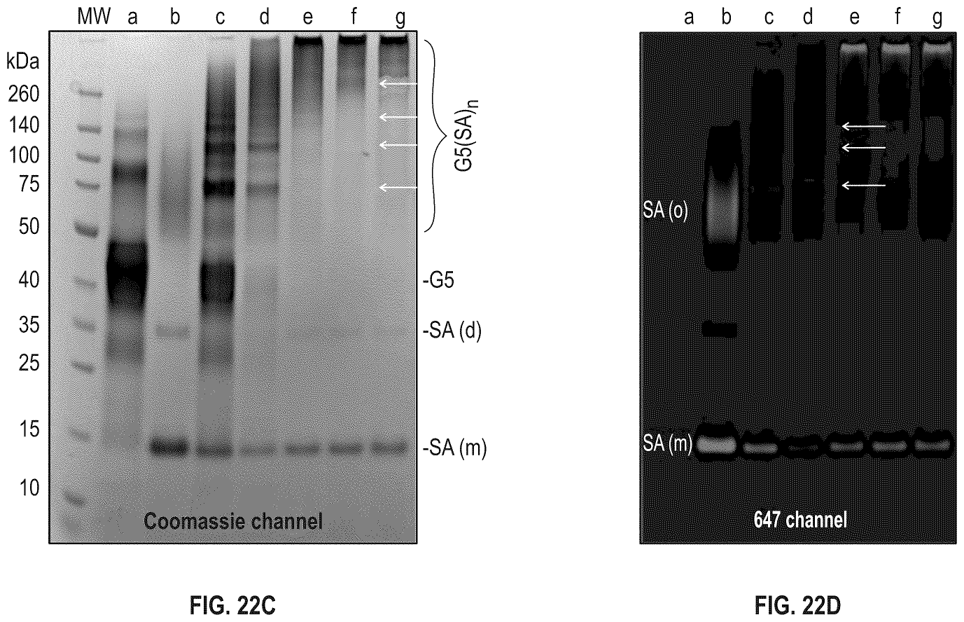

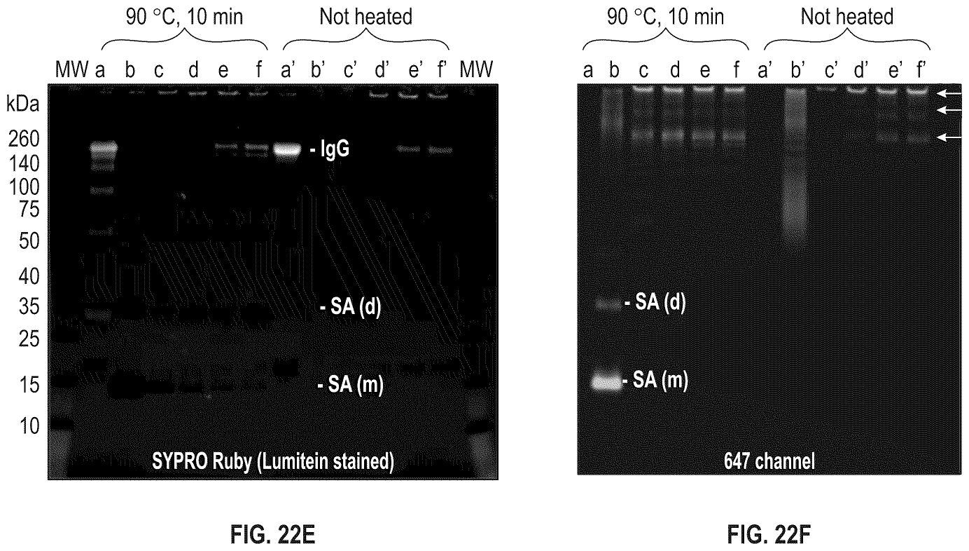

This example describes conjugation of G5(aldehyde) dendrimer with streptavidin(hydrazine) which leads to the synthesis of G5(SA) (FIG. 22A).

First, streptavidin (SA) was modified with AZ647 dye and HyNic (hydrazine) in a one-pot reaction to prepare its conjugate SA(HyNic)(AZ647). In a micro vial which contained streptavidin (ThermoFisher Scientific) dissolved in BBS pH 8.4 (0.5 mL, 0.597 mg/mL) was added AZ647 NHS ester (27.4 μL, 1.05 mM; 3 eq). This mixture was vortexed and shaken at 25° C. After 0.5 h, it was added with a first portion (6 eq) of 6-hydrazinonicotinate acetone hydrazone (HyNic)-PEG4 NHS ester (3.1 μL, 18.6 mM) dissolved in 10% DMSO/MeCN. This mixture was shaken at 25° C. for 1 h prior to adding a second portion of HyNic-PEG4 NHS ester (3.1 μL, 18.6 mM; 6 eq). This final mixture was shaken overnight and treated with 10% aqueous acetic acid (5 μL). After shaking for 1 h. the reaction mixture was purified by spin filtration in which the mixture was loaded into a membrane tube (Sartorius, Vivaspin® MWCO 10,000; 2.0 mL capacity) and diluted with PBS pH 6.5 to 2.0 mL. It was centrifuged at 6,000 (4200 g) for 12 min. After discarding its filtrate, its centrifugal filtration was repeated three more times using PBS pH 6.5. Its concentrate was collected by centrifugation and its volume was adjusted to 0.4 mL (19.2 μM) with PBS pH 6.5, affording SA(HyNic)(AZ647).

Conjugation of AZ647 and HyNic moieties in SA was confirmed by UV-vis analysis which showed absorbance at λₘₐₓ of 650 nm which is indicative of AZ647 conjugation and an increase in absorbance at 280 nm which is contributed by HyNic attachment. Its analysis in SEC-HPLC shows a single peak at tᵣ of 14.4 min, suggesting its homogeneity. Size exclusion chromatography (SEC) HPLC was performed as set forth in Example XIII.

Second, SA(HyNic)(AZ647) was conjugated with G5(Ald)₅₀ through a hydrazone linkage. A micro vial was loaded with PBS pH 7.4 (0.19 mL), 2-amino-5-methoxybenzoic acid (AMBA) (0.125 mL, 100 mM) in 10% DMSO in PBS pH 7.4 and SA(HyNic)(AZ647) (0.25 mL, 19.2 μM). This vial was vortexed prior to adding a solution of G5(Ald) (60.5 μL, 12.3 μM; 6.5 molar eq of SA to G5). This mixture was vortexed and shaken at 25° C. for 15 h. For its purification, the reaction mixture was loaded into a membrane tube (Sartorius, Vivaspin® MWCO 10,000; 2.0 mL capacity) and diluted with PBS pH 7.4 to 2.0 mL. It was centrifuged at 6,000 (4200 g) rpm for 12 min. After discarding its filtrate, its centrifugal filtration was repeated three more times using PBS pH 7.4. Its concentrate was collected by centrifugation and its volume was adjusted to 0.3 mL with PBS pH 7.4, affording G5(SA)_{n=6.5}([G5]=2.0 μM).

Formation of conjugate G5(SA)_{n=6.5} between G5(Ald) and SA(HyNic) is supported by UV-vis (nanodrop) and SEC-HPLC analysis. Its UV-vis spectral trace shows a new broad peak at λₘₐₓ of 330 nm, which is attributed to the functionality of their hydrazone linkage. Its SEC-HPLC analysis (FIG. 22B) shows a broad peak (11.5-15 min), which is distinct in peak shape and retention time (tᵣ) from its individual components, each displaying a relatively sharp peak at tᵣ of 14.6 min and 14.4 min, respectively. Formation of G5(SA)n is also evident in its SDS-PAGE gel analysis, which was performed as set forth in Example XII and is presented in FIGS. 22C and 22D. Its conjugate species consists of several bands migrating much higher in MW compared to its individual components G5(Ald)₅₀ and SA(647).

### Example XVIII

### Preparation of IgG(Biotin) Complexes with G5(SA)

This example describes a method of preparing a binary complex of G5(SA) with biotin-modified IgG as depicted in FIG. 22A.

G5(SA)₆ (50 μL, 1.97 μM) was loaded into a micro vial and mixed with a solution of anti-DTR IgG(biotin) (53.7 μL, 5.5 μM) (EXAMPLE XV). These were complexed in a molar ratio of 3 IgG to 1 G5(SA). This mixture was vortexed and shaken at 25° C. for 0.5 h, yielding a complex G5(SA)@IgG(biotin).

Formation of IgG(biotin) complexes with G5(SA)₆ is supported by its SDS-PAGE gel analysis, performed as described in Example XII, with results presented in FIGS. 22E and 22F. Its complexes are evident with new bands migrating higher in MW compared to its individual components IgG(biotin) and G5(SA).

### Example XIX

### Preparation of G5(protein A) Conjugate Through Hydrazone Linkage

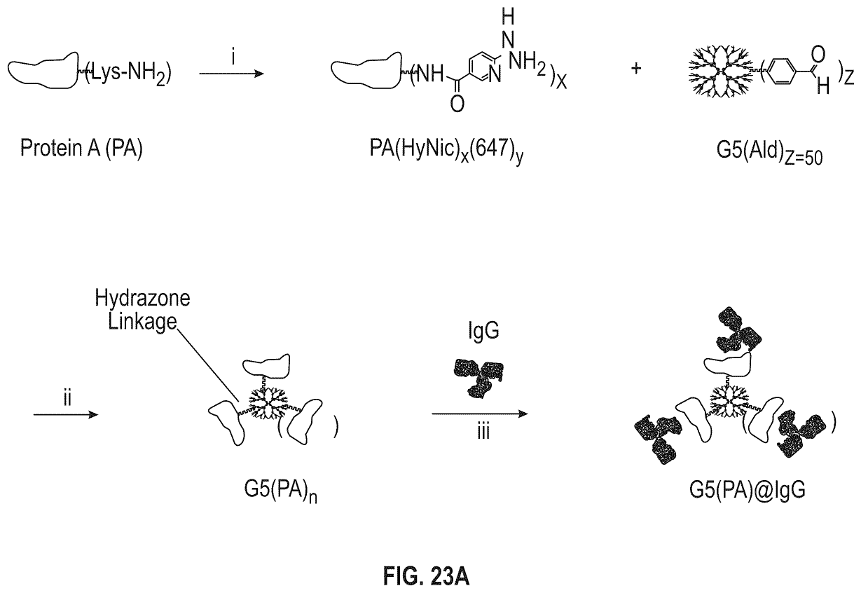

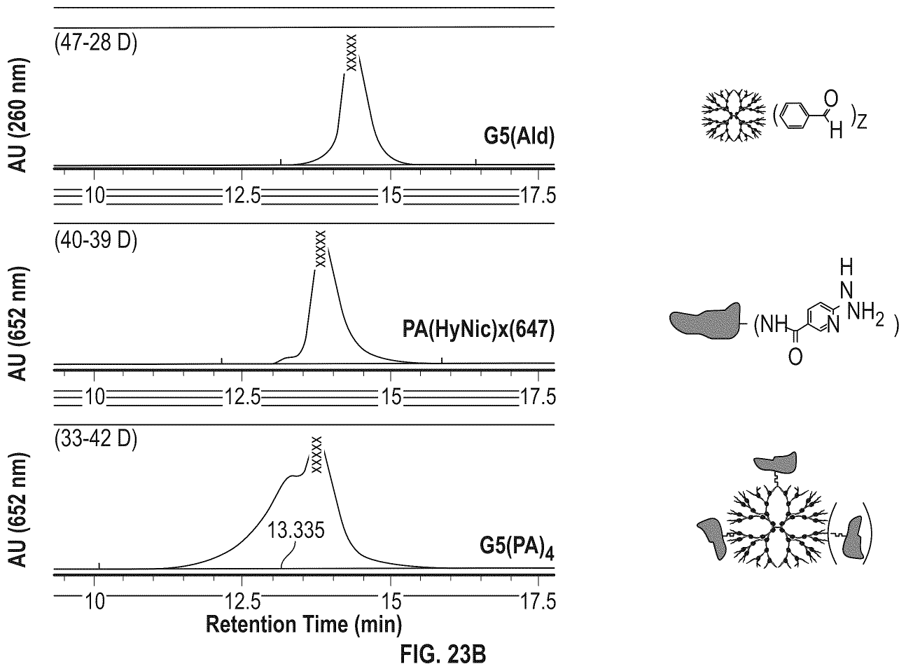

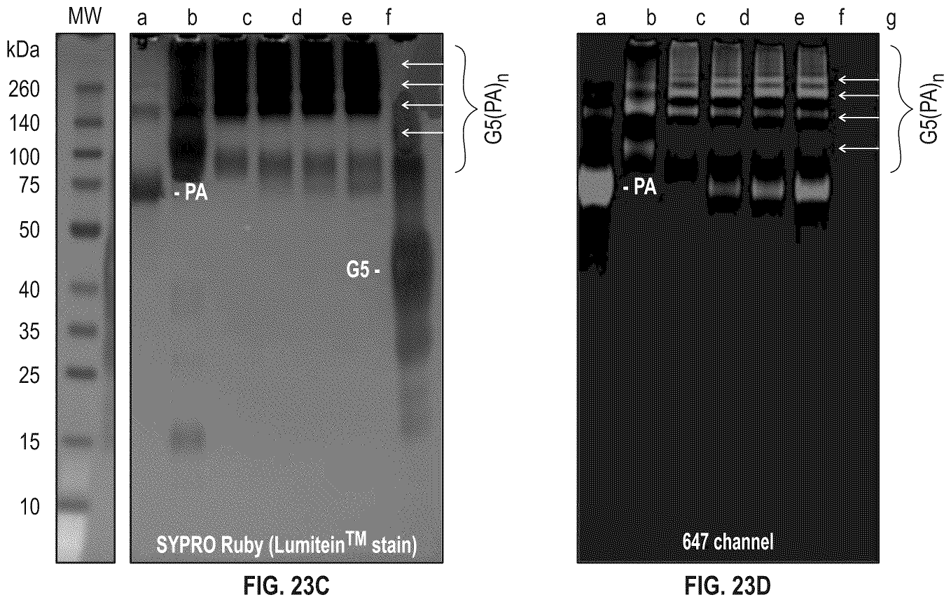

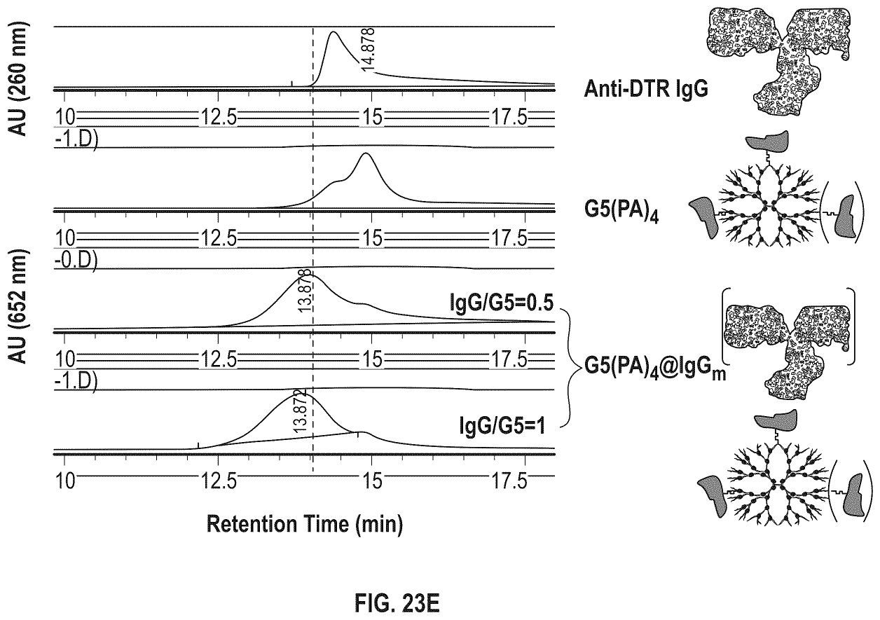

This example describes a method of conjugating G5(aldehyde) dendrimer with protein A(hydrazine) which leads to the synthesis of G5(protein A) (FIG. 23A).

First, protein A (PA) was modified with AZ647 dye and HyNic (hydrazine) in a one-pot reaction to prepare its conjugate PA(HyNic)ₓ(AZ647)y. In a micro vial which contained streptavidin (SpA, Sigma-Aldrich) dissolved in BBS pH 8.4 (0.5 mL, 1.0 mg/mL) was added AZ647 NHS ester (45.5 μL, 1.05 mM; y=4 eq). This mixture was vortexed and shaken at 25° C. After 0.5 h, it was added with a first portion (x=6 eq) of 6-hydrazinonicotinate acetone hydrazone (HyNic)-PEG4 NHS ester (3.8 μL, 18.6 mM) dissolved in 10% DMSO/MeCN. This mixture was shaken at 25° C. for 1.5 h prior to adding a second portion of HyNic-PEG4 NHS ester (3.8 μL, 18.6 mM; x=6 eq). This final mixture was shaken overnight and treated with 10% aqueous acetic acid (5 mL). After shaking for 1 h., the reaction mixture was purified by spin filtration in which the mixture was loaded into a membrane tube (Sartorius, Vivaspin® MWCO 10,000; 2.0 mL capacity) and diluted with PBS pH 6.5 to 2.0 mL. It was centrifuged at 6,000 (4200 g) rpm for 12 min. After discarding its filtrate, its centrifugal filtration was repeated three more times using PBS pH 6.5. Its concentrate was collected by centrifugation and its volume was adjusted to 0.4 mL (23.8 μM) with PBS pH 6.5, affording PA(HyNic)ₓ(AZ647)y.

Conjugation of AZ647 and HyNic moieties in PA was confirmed by UV-vis analysis which showed absorbance at λₘₐₓ of 650 nm which is indicative of AZ647 conjugation (average 2.9 dye molecules per PA) and an increase in absorbance at 280 nm which is contributed by HyNic attachment. Its analysis in SEC-HPLC, performed as set forth in Example XIII, showed a single peak at tᵣ of 13.9 min, indicating homogeneity. Second, PA(HyNic)(AZ647) was conjugated with G5(Ald)₅₀ through a hydrazone linkage. A micro vial was loaded with PBS pH 7.4 (0.117 mL), 2-amino-5-methoxybenzoic acid (AMBA) (0.150 mL, 100 mM) in 10% DMSO in PBS pH 7.4 and PA(HyNic)(AZ647) (0.3 mL, 23.8 mM). This vial was vortexed prior to adding a solution of G5(Ald) (145 μL, 12.3 μM; 4 molar eq of PA to G5). This mixture was vortexed and shaken at 25° C. for 15 h. For its purification, the reaction mixture was loaded into a membrane tube (Sartorius, Vivaspin® MWCO 10,000; 2.0 mL capacity) and diluted with PBS pH 7.4 to 2.0 mL. It was centrifuged at 6,000 (4200 g) rpm for 12 min. After discarding its filtrate, its centrifugal filtration was repeated three more times using PBS pH 7.4. Its concentrate was collected by centrifugation and its volume was adjusted to 0.3 mL with PBS pH 7.4, affording G5(PA)ₙ₌₄ ([G5]=4.8 mM).

Formation of conjugate G5(PA)ₙ₌₄ between G5(Ald) and PA(HyNic) is supported by UV-vis (nanodrop) and SEC-HPLC analysis. Its UV-vis spectral trace shows a new broad peak at λₘₐₓ of 330 nm, which is attributed to the functionality of their hydrazone linkage. Its SEC-HPLC analysis (FIG. 23B) shows a broad peak (11.5-15 min), which is distinct in peak shape and retention time (tᵣ) from its individual components, each displaying a relatively sharp peak at tᵣ of 14.4 min and 13.9 min, respectively. Formation of G5(PA)ₙ₌₄ is also evident in its SDS-PAGE gel analysis as presented in FIGS. 23C and 23D. Its conjugate species consists of several new bands, each migrating much higher in MW compared to its individual components G5(Ald)₅₀ and PA(HyNic).

### Example XX

### Preparation of IgG Complexes with G5(PA)

This example describes a method for preparing an IgG complex with G5(PA)ₙ₌₄ as depicted in FIG. 23A.

G5(PA)₄ (50 μL, 4.76 μM) was loaded into a micro vial and mixed with a solution of anti-DTR IgG (107 μL, 6.67 μM). These were mixed in a molar ratio of 3 IgG to 1 G5(PA). This mixture was vortexed and shaken at 25° C. for 0.5 h, yielding an anti-DTR IgG complex G5(PA)@IgG ([IgG]=4.4 mM). The formation of IgG complexes with G5(PA)₄ is supported by its SEC-HPLC analysis, performed as set forth in Example XIII, and results as presented in FIG. 23E. Its complex trace shows a new peak which is broader in peak shape and faster in retention time (tᵣ) relative to its individual components. Such elution features are accounted for by an increase in molecular size as anticipated when complexes are formed.

### Example XXI

### Preparation of G5(IgG) Conjugate Through Hydrazone Linkage

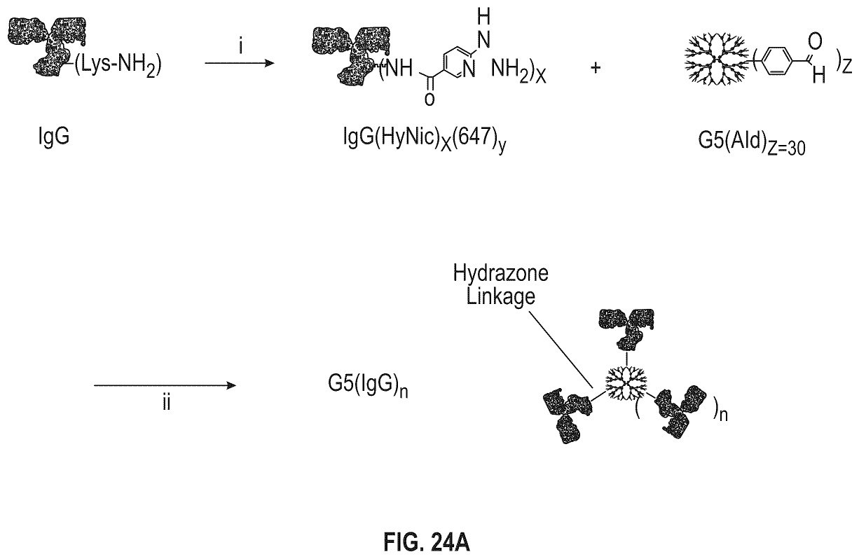

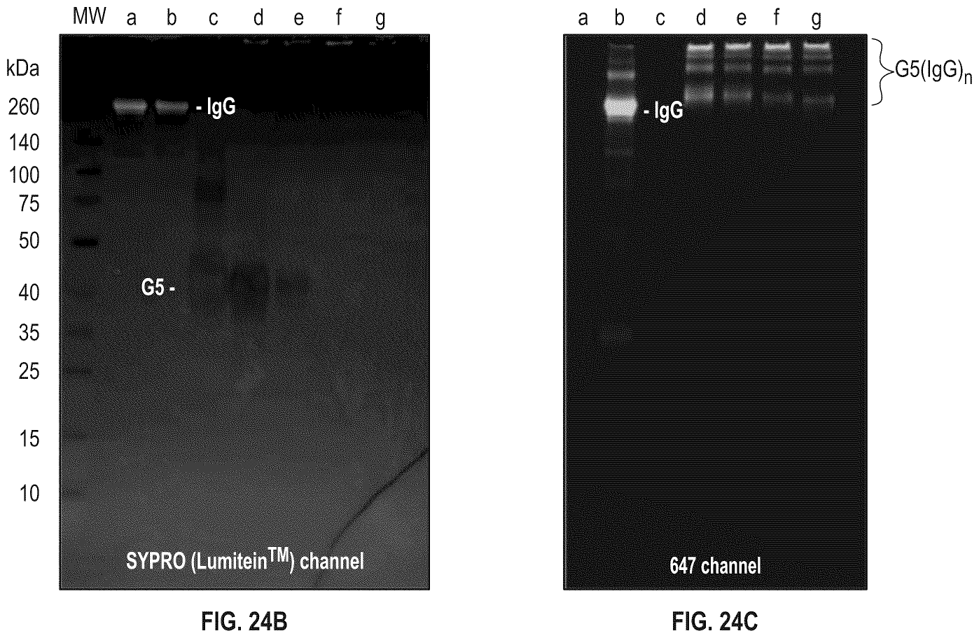

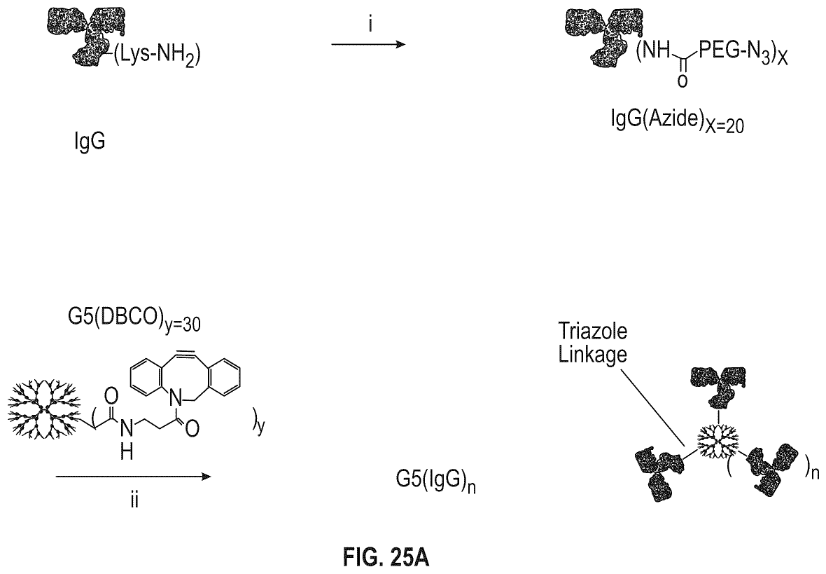

This example describes a method for conjugating G5(aldehyde) dendrimer with IgG(hydrazine) which leads to the synthesis of G5(IgG) (FIG. 24A).

First, IgG was modified with AZ647 dye and HyNic (hydrazine) in a one-pot reaction to prepare its conjugate IgG(HyNic)ₓ(AZ647)y. In a micro vial which contained anti-DTR IgG dissolved in BBS pH 8.4 (0.5 mL, 1.0 mg/mL) was added AZ647 NHS ester (15.9 μL, 1.05 mM; y=6 eq). This mixture was vortexed and shaken at 25° C. After 0.5 h, it was added with a first portion (x=20 eq) of 6-hydrazinonicotinate acetone hydrazone (HyNic)-PEG4 NHS ester (3.0 mL, 18.6 mM) dissolved in 10% DMSO/MeCN. This mixture was shaken at 25° C. for 2.5 h prior to adding a second portion of HyNic-PEG4 NHS ester (3.0 μL, 18.6 mM; x=20 eq). This final mixture was shaken overnight and treated with 10% aqueous acetic acid (5 μL). After shaking for 1 h. the reaction mixture was purified by spin filtration in which the mixture was loaded into a membrane tube (Sartorius, Vivaspin® MWCO 10,000; 2.0 mL capacity) and diluted with PBS pH 6.5 to 2.0 mL. It was centrifuged at 6,000 (4200 g) rpm for 12 min. After discarding its filtrate, its centrifugal filtration was repeated three more times using PBS pH 6.5. Its concentrate was collected by centrifugation and its volume was adjusted to 0.4 mL (5.6 μM) with PBS pH 6.5, affording anti-DTR IgG(HyNic)ₓ(AZ647)y.

Conjugation of AZ647 and HyNic moieties in IgG was confirmed by UV-vis analysis which showed absorbance at λₘₐₓ of 650 nm which is indicative of AZ647 conjugation (average 3.0 dye molecules per IgG) and an increase in absorbance at 280 nm which is contributed by HyNic attachment.

Second, the IgG(HyNic)(AZ647) was then conjugated with G5(Ald)₅₀ through hydrazone linkage chemistry. A micro vial was loaded with PBS pH 7.4 (0.70 mL), 2-amino-5-methoxybenzoic acid (AMBA) (0.25 mL, 100 mM) in 10% DMSO in PBS pH 7.4 and anti-DTR IgG(HyNic)(AZ647) (0.25 mL, 5.56 μM). This vial was vortexed prior to adding a solution of G5(Ald) (3.8 μL, 12.3 μM; 3 molar eq of IgG to G5). This mixture was vortexed and shaken at 25° C. for 15 h. For its purification, the reaction mixture was loaded into a membrane tube (Sartorius, Vivaspin® MWCO 10,000; 2.0 mL capacity) and diluted with PBS pH 7.4 to 2.0 mL. It was centrifuged at 6,000 (4200 g) rpm for 12 min. After discarding its filtrate, its centrifugal filtration was repeated three more times using PBS pH 7.4. Its concentrate was collected by centrifugation and its volume was adjusted to 0.23 mL with PBS pH 7.4, affording G5(anti-DTR IgG)ₙ₌₃ ([IgG]=1.3 μM).

Formation of conjugate G5(IgG)ₙ₌₃ between G5(Ald) and IgG(HyNic)(AZ647) is supported by UV-vis (nanodrop) and SDS-PAGE analysis, which was performed as set forth in Example XII. Its UV-vis spectral trace shows a new peak at λₘₐₓ of 350 nm (310-400 nm), which is attributed to the functionality of their hydrazone linkage. Formation of G5(IgG)ₙ₌₃ is also evident in its SDS-PAGE gel analysis as presented in FIGS. 24B and 24C. It displays several new bands, each migrating higher in MW compared to its individual components G5(Ald)₅₀ and IgG(HyNic)(AZ647).

### Example XXII

### Preparation of G5(IgG) Conjugate Through Azide-DBCO Click Chemistry

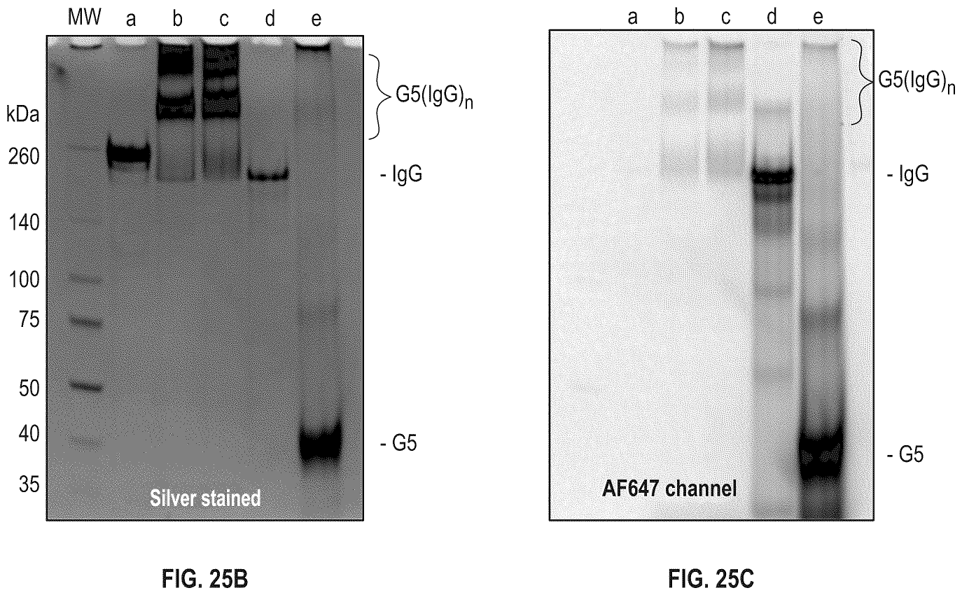

This example describes a method of conjugating G5(DBCO) dendrimer with IgG(azide) which leads to the synthesis of G5(IgG) (FIG. 25A).

First, IgG was modified to prepare its azide conjugate IgG(azide). In a micro vial which contained anti-DTR IgG dissolved in BBS pH 8.4 (0.5 mL, 1.0 mg/mL) was added a solution of azide-PEG4 TFP ester prepared in acetonitrile (30.5 μL, 2.28 mM; x=20 eq). This mixture was vortexed and shaken at 25° C. After 2 h, it was added with a second portion (x=20 eq) of azide-PEG4 TFP ester in acetonitrile. This mixture was shaken at 25° C. overnight and the reaction mixture was purified by spin filtration for which the mixture was loaded into a membrane tube (Sartorius, Vivaspin® MWCO 10,000; 2.0 mL capacity) and diluted with PBS pH 7.4 to 2.0 mL. It was centrifuged at 6,000 (4200 g) rpm for 15 min. After discarding its filtrate, its centrifugal filtration was repeated three more times using PBS pH 7.4. Its concentrate was collected by centrifugation and its volume was adjusted to 0.4 mL (5.2 μM) with PBS pH 7.4, affording anti-DTR IgG(azide)ₓ.

Second, IgG(azide) was then conjugated with G5(DBCO)₃₀ through strain-promoted azide-alkyne click (SPAAC) chemistry. A micro vial was loaded with G5(DBCO) (3.2 μL, 41.5 mM) and anti-DTR IgG(azide) (50 μL, 5.2 μM; 2 eq to G5). This vial was shaken at 25° C., and after 15 h, its mixture was treated with azide-AF647 (2.5 μL, 1.05 mM; 20 eq to G5). This final mixture continued shaken at 25° C. for 15 h when it was diluted with PBS pH 7.4 (0.14 mL) and purified by passing through a desalting column (Zeba™ MWCO 7,000) at 4000 rpm (1500 g) for 2 min. This afforded AF647-labeled G5(anti-DTR IgG)ₙ₌₃ ([IgG]=0.94 μM; #AF647 per IgG=8).

Conjugation of azide moieties in IgG was confirmed by its click conjugation with DBCO-AF647 and analysis by analysis with UV-vis and SDS-PAGE as set forth in Example XII. As shown in FIG. 25C, its gel band showed fluorescence at 647 channel, evidence which is indicative of DBCO-AZ647 conjugation at azide-modified IgG. Its UV-vis absorbance at 647 nm by nanodrop suggests an average number of 8 dye molecules conjugated per IgG.

Formation of conjugate G5(IgG)ₙ₌₃ between G5(DBCO)₃₀ and IgG(azide) is evident in SDS-PAGE gel analysis. As presented in FIGS. 25B and 25C, it displays formation of new bands, each migrating higher in MW compared to its individual components G5(DBCO)₃₀ and IgG(azide).

### Example XXIII

### On-Array Detection Assay

This example describes an assay that measures detection efficiency of a dendrimer Lobe (anti-DTR IgG-SA-G5 complex) in its binding to DTR peptides presented on a streptavidin-SNAP performed on an array in a microfluidic system observed by a fluorescence microscope.

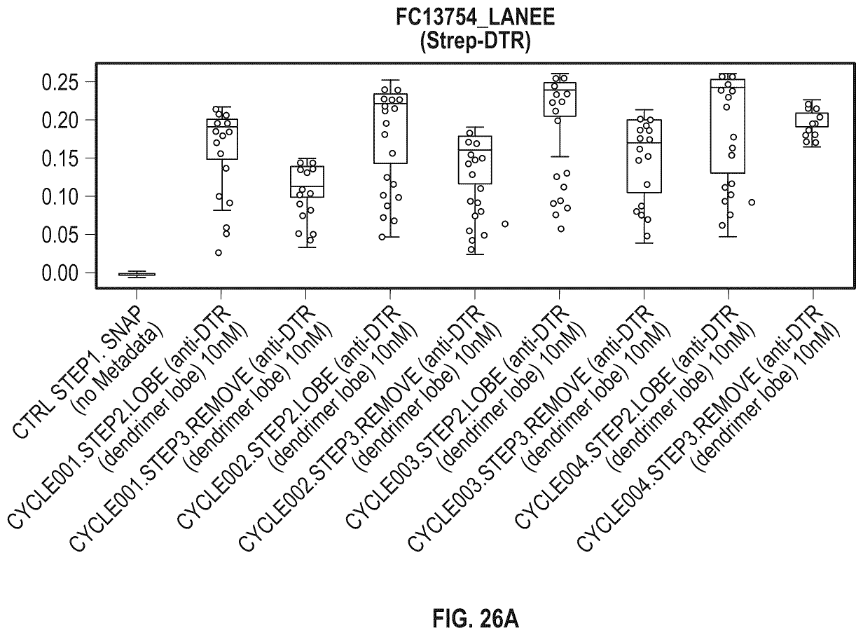

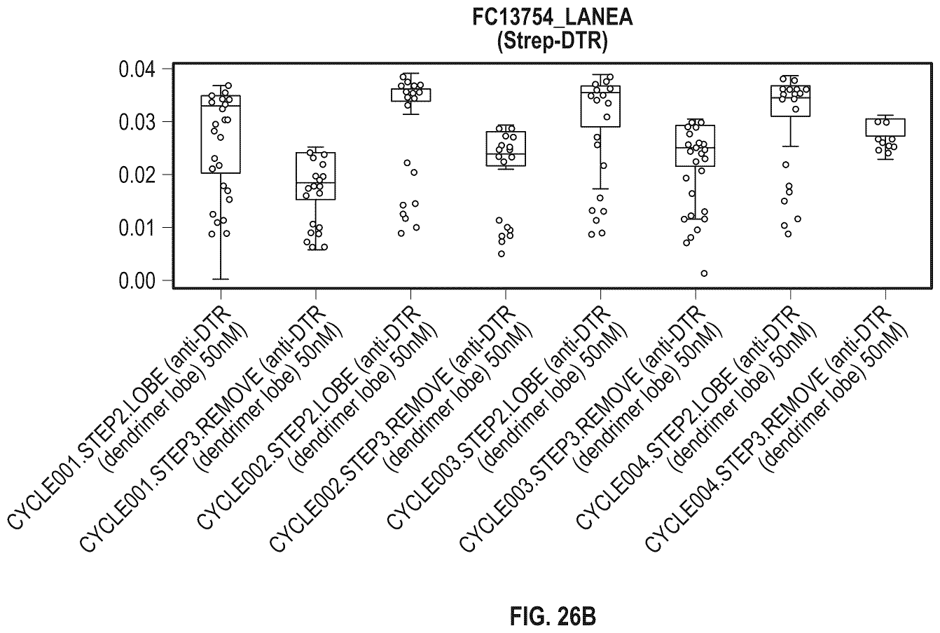

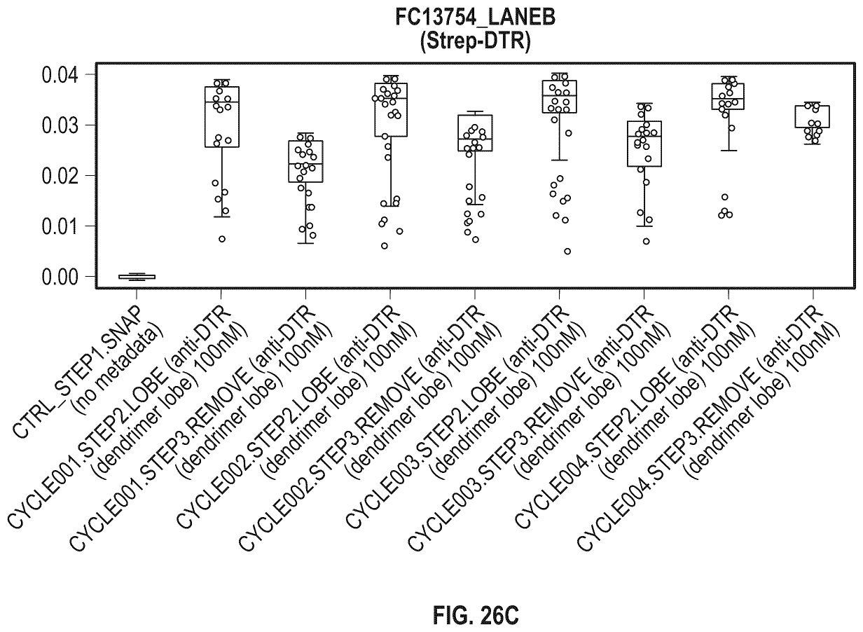

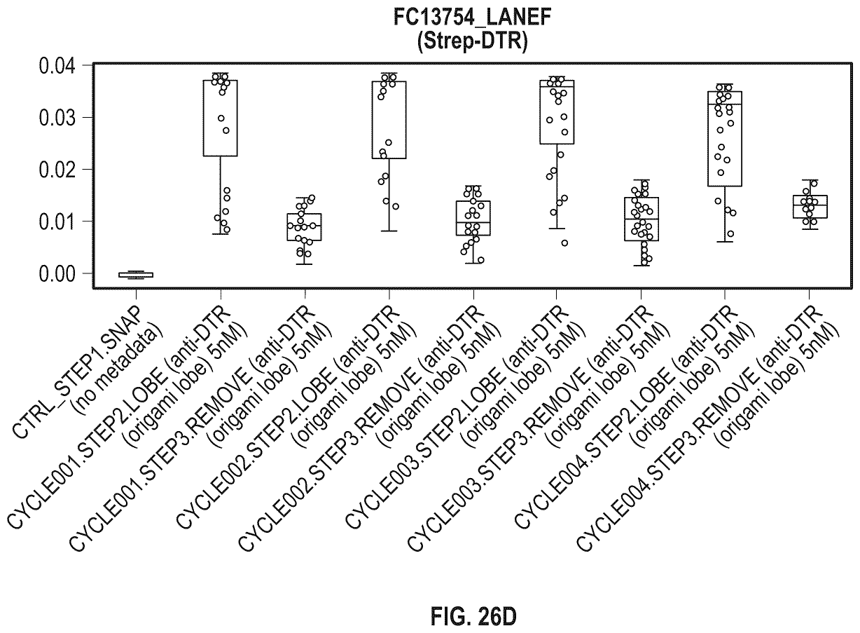

An aliquot of a peptide-presented SNAP solution (150 μM) was loaded in a flow cell lane which was pre-coated with a SNAP-complementary, single stranded oligonucleotide on the surface. This flow cell was incubated for 30 min and flowed with a buffer solution to remove unbound SNAP. Each lobe solution was then loaded in the flow cell lanes at either 50-100 nM for dendrimer lobes or at 5-10 nM for origami lobes as indicated in the plots of FIG. 26. After incubation for 0.5 h, the flow cell was washed to remove unbound Lobe by buffer flow and scanned for its fluorescent emission at 488 nm for SNAP and at 647 nm for Lobe. Image analysis was performed to determine the locations of individual SNAPs deposited (fluorescence at 488 nm) and those of Lobe bound (fluorescence at 647 nm). Their rate of colocalization, which is referred to as the fraction of Lobe bound per SNAP, is used to calculate % detection efficiency.

### Example XXIV

### Fluorescence-Linked Immunosorbent Assay (FLISA)

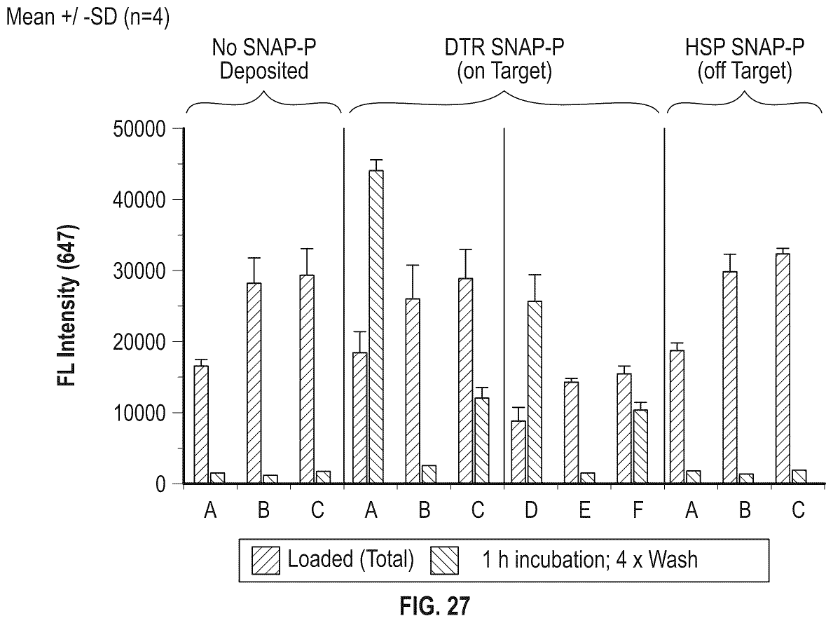

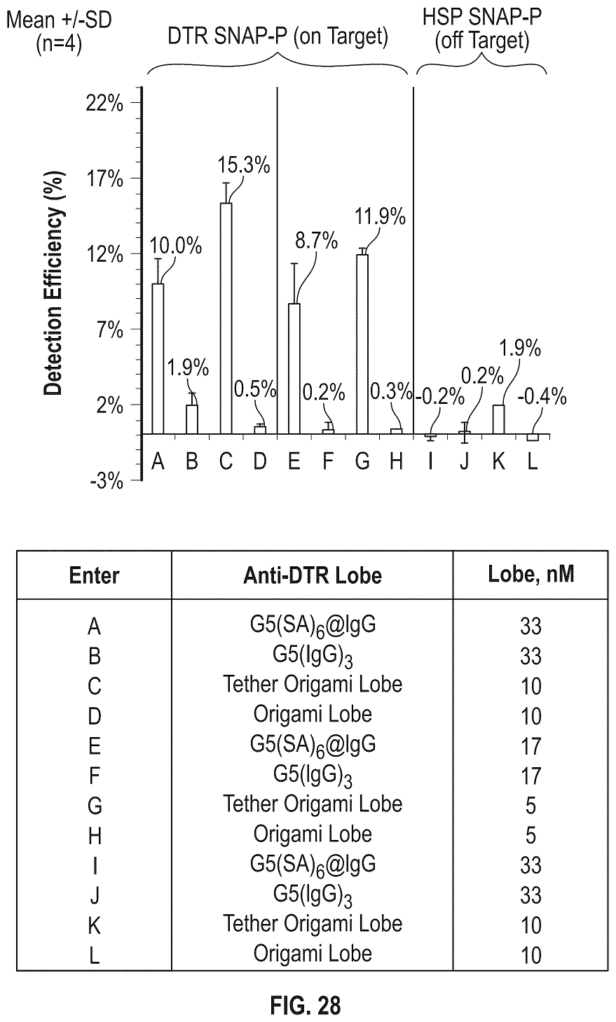

This example describes an assay method that measures binding efficiency of anti-DTR IgG-dendrimer Lobes to SNAP presenting either multiple (1-3) peptides (streptavidin-SNAP; FIG. 27) or a single peptide (TCO-SNAP; FIG. 28).

An aliquot of a peptide-presented SNAP solution was loaded in quadruplicate in microplate wells which were pre-coated with a single stranded oligonucleotide on the surface. This oligonucleotide sequence is complementary to an oligonucleotide co-presented on the SNAP-peptide. Hybridization between the oligonucleotides allows the SNAP to be immobilized on the well surface. This plate was incubated for 2 h and washed with a buffer solution (10 mM HEPES, 120 mM NaCl, 10 mM MgCl₂, 5 mM KCl, 0.1% tween 20, 0.001% lipidure) four times to fully remove excess, unbound SNAP. This SNAP-deposited plate was then inserted in a multimode microplate reader (Tecan, Spark@) and scanned for its fluorescent emission at 488 nm using automatic adjustment for optimal gain. After scans, the SNAP wells were treated with each Lobe solution at either 50-100 nM for dendrimer lobes or at 5-10 nM for origami lobes as indicated in the plots of FIG. 27 and FIG. 28. The plate was incubated for 1-1.5 h, washed with the buffer four times, and scanned for its fluorescent emission at 647 nm under an optimal gain condition. In a separate experiment, fluorescence measurements for each SNAP alone and Lobe alone were made to establish their standard curves of fluorescence against concentration in a linear dynamic range of 4-63 μM (SNAP), 1-16 μM (origami Lobe) or 2-390 μM (dendrimer Lobe; IgG basis). Fluorescence analysis was performed to determine the amounts (concentrations) of SNAP deposited (fluorescence at 488 nm) and Lobe bound (fluorescence at 647 nm). Their quantitation allowed calculating Lobe detection efficiency, which is the ratio of [Lobe] to [SNAP]. Detection efficiency for each Lobe was presented as an average value ±standard deviation (SD; n=4).

What is claimed is:

1. A method of detecting a polypeptide, comprising
(a) providing an affinity reagent comprising an artificial polymer comprising a branched chain that is attached to an affinity moiety and a label;
(b) binding the affinity moiety of the affinity reagent to a polypeptide; and
(c) detecting the label, thereby detecting binding of the affinity reagent to the polypeptide.

2. The method of claim 1, wherein the polypeptide is attached to a solid support.

3. The method of claim 2, wherein the solid support comprises an array of polypeptides.

4. The method of claim 3, wherein the detecting of the label individually resolves the polypeptide from all other polypeptides in the array of polypeptides.

5. The method of claim 1, wherein the artificial polymer is selected from the group consisting of polyethylenimine (PEI), polypropyleneimine (PPI), poly(amidoamine) (PAMAM), PAMAM dendron, and poly-L-lysine (PLL).

6. The method of claim 1, wherein the affinity moiety is selected from the group consisting of an antibody, an antibody fragment, and a nucleic acid aptamer.

7. The method of claim 1, wherein the detecting of step (c) comprises detecting an optical signal from the label.

8. The method of claim 1, wherein at least two affinity moieties are attached to the artificial polymer.

9. The method of claim 8, wherein the at least two affinity moieties recognize the same epitope in the polypeptide.

10. The method of claim 8, wherein the at least two affinity moieties recognize different epitopes in the polypeptide.

11. The method of claim 1, wherein at least two labels are attached to the artificial polymer.

12. The method of claim 11, wherein the detecting of step (c) comprises simultaneously detecting the at least two labels.

13. The method of claim 1, further comprising (d) dissociating the affinity moiety of the affinity reagent from the polypeptide, thereby removing the affinity reagent from the polypeptide.

14. The method of claim 1, wherein the affinity moiety is covalently attached to the artificial polymer.

15. The method of claim 1, wherein the label is covalently attached to the artificial polymer.

16. A molecule, comprising
(a) an artificial polymer comprising a branched chain;
(b) an affinity moiety attached to the artificial polymer; and
(c) a label attached to the artificial polymer.

17. A kit, comprising
(a) a first affinity reagent comprising a first artificial polymer attached to a first affinity moiety and a first label, wherein the first artificial polymer comprises a branched chain structure; and
(b) a second affinity reagent comprising a second artificial polymer attached to a second affinity moiety and a second label, wherein the second artificial polymer comprises the branched chain structure,
wherein the first affinity moiety recognizes an epitope that is not recognized by the second affinity moiety and the second affinity moiety recognizes an epitope that is not recognized by the first affinity moiety.

18. A method of making an affinity reagent comprising
(a) providing an artificial polymer comprising a branched chain, wherein at least one chain of the artificial polymer comprises an alkyne moiety; and
(b) reacting a cysteine of an antibody with the alkyne moiety, thereby covalently attaching the antibody to the artificial polymer.

19. A method of making an affinity reagent comprising
(a) providing an artificial polymer comprising a branched chain, wherein at least one chain of the artificial polymer comprises a first member of a receptor-ligand pair;
(b) providing an affinity component comprising a second member of the receptor-ligand pair; and
(c) binding the first and second members of the receptor-ligand pair, whereby the affinity component forms an affinity moiety of the artificial polymer.
